(19) United States
(12) Patent Application Publication
(10) Pub. No.: US 2016/0000762 A1
(43) Pub. Date: Jan. 7, 2016

(54) IMPROVING COGNITIVE FUNCTION

(71) Applicants: THE REGENTS OF THE UNIVERSITY OF CALIFORNIA, Oakland, CA (US); GEORGETOWN UNIVERSITY, Washington, DC (US)

(72) Inventors: Jerry Yang, La Jolla, CA (US); Hyang-Sook Hoe, Daegu (KR); Raymond Scott Turner, Washington, DC (US)

(21) Appl. No.: 14/771,118

(22) PCT Filed: Feb. 27, 2014
(86) PCT No.: PCT/US14/18966

§ 371 Date: Aug. 27, 2015

Related U.S. Application Data
(60) Provisional application No. 61/770,078, filed on Feb. 27, 2013.

(51) Int. Cl. A61K 31/428

(52) U.S. Cl. CPC A61K 31/428

(57) ABSTRACT

Provided herein, inter alia, are compounds useful in improving cognitive function, memory and learning in both healthy and diseased subjects.

### CROSS-REFERENCES TO RELATED APPLICATIONS

This application claims the benefit of U.S. Provisional Application No. 61/770,078, filed Feb. 27, 2013, the content of which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under Grant Nos. AI077644 and GM098435 awarded by the National Institutes of Health. The government has certain rights in the invention.

### BACKGROUND OF THE INVENTION

There is a need in the art to increase cognitive function, memory and learning in both healthy and diseased subjects. Dendritic complexity, synaptogenesis, and overall proper development and function of neurons are regulated by growth factors collectively called neurotrophins. Promotors of proper spinogenesis in primary neurons (in vitro and in vivo) are useful for a variety of purposes including improving memory and learning in animals. Provided herein, inter alia, are solutions to these and other problems in the art using benzothiazole aniline (BTA) compounds.

### BRIEF SUMMARY OF THE INVENTION

In a first aspect, there is provided a method for improving memory or learning in a subject in need thereof, the method including administering to the subject an effective amount of a compound of Formula (I):

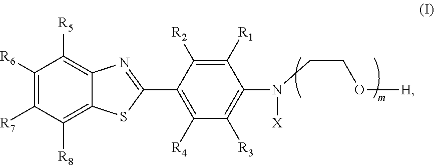

wherein R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl; m is a integer in the range 1-20; and X is hydrogen, methyl, or ethyl.

In another aspect, there is provided a method for treating neuronal or cognitive impairment in a subject in need thereof, the method including administering to the subject an effective amount of a compound of Formula (I) as disclosed herein, and embodiments thereof.

In another aspect, there is provided a method of increasing dendritic spine formation, increasing dendritic spine density or improving dendritic spine morphology in a subject in need thereof, the method including administering to the subject an effective amount of a compound of Formula (I), as disclosed herein, and embodiments thereof.

In another aspect, there is provided a method of increasing functional synapses in a subject in need thereof, the method including administering to the subject an effective amount of a compound of Formula (I), as disclosed herein, and embodiments thereof.

In another aspect, there is provided a method of increasing functional synapses in a subject in need thereof, the method including administering to the subject an effective amount of a compound of Formula (I), as disclosed herein, and embodiments thereof.

### BRIEF DESCRIPTION OF THE DRAWINGS

FIGS. 1A-1L. BTA-EG₄ exhibits low toxicity and crosses the blood-brain barrier in vivo. FIG. 1A: Structure of BTA-EG₄ (top) and time-dependent plasma (boxes) and brain (circles) concentrations of BTA-EG₄ in wild-type mice that were injected (10 mg/kg, i.p.; n=2 per time point). FIG. 1B: Table summarizing the calculated pharmacokinetic parameters for the plasma and brain profile of BTA-EG₄. Parameters include the t_{1/2} for BTA-EG₄ in the plasma and brain, the Cₘₐₓ of BTA-EG₄ in the plasma and brain, the area under the curve (AUC), the brain-to-plasma ratio (BB), and the Log BB. FIG. 1C: Primary cortical neuronal cells were treated with control, or 1 or 5 μM BTA-EG₄ for 24 h. Aβ levels in the conditioned media were determined by ELISA (n=12/group). FIG. 1D: Histogram of wild-type mice injected (“B”) with 15 mg/kg, i.p., BTA-EG₄ (left columns) or 30 mg/kg BTA-EG₄ daily (right columns) for 2 weeks; control (“C”). Aβ levels were measured in the brain (n=12/group). FIGS. 1E-1F: Wild-type mice were injected with 30 mg/kg BTA-EG₄ for 2 weeks and sAPPα (FIG. 1E), sAPPβ (FIG. 1E), full-length APP (FIG. 1F), APP C-terminal fragment (CTF) (FIG. 1E), and β-actin (FIGS. 1E-1F) were measured (n=3). FIG. 1G: Histogram of quantification of sAPPα and sAPP_from FIG. 1E and FIG. 1F (n=3/group). The sAPPα and sAPPβ signals for each sample were normalized to β-actin. FIGS. 1H-1I: COS7 cells expressing APP (FIG. 1H, n=3) or primary cortical neurons (FIG. 1I, n=3) were treated with BTA-Ea₄ (5 μM) for 24 h. Cell surface proteins were biotinylated, isolated with avidin-conjugated beads, and immunoblotted with 6E10 or 22C11 antibody. FIG. 1J: Cultured hippocampal neurons (DIV18) were transfected with GFP and APP and treated with BTA-EG₄ for 24 h, and live cell surface staining was conducted. Left panels, GFP; right panels, surface APP (n=10/group). FIG. 1K: Cultured hippocampal neurons (DIV18) were transfected with GFP and APP, treated with BTA-EG₄ for 24 h, and immunostained with anti-APP. Left panels, GFP; right panels, total APP (n=10/group). FIG. 1L: Histogram of quantification of surface APP intensity from FIG. 1J and total APP from FIG. 1K. *p=0.05, **p=0.01, ***p=0.001. C, Control; B, BTA-EG₄.

FIGS. 2A-2F. BTA-EG₄ improves cognitive performance. FIGS. 2A-2D: Spatial learning task for BTA-EG₄-injected (intraperitoneally) wild-type mice by Morris water maze paradigm. FIG. 2A: Escape latencies during the training phase (n=15; *p=0.05 for days 2 and 4; p=0.001 overall). Control (solid circle); BTA-EG₄ (open boxes). FIG. 2B: Path lengths to the platform during training trials (two-way ANOVA, p=0.6709). FIG. 2C: Histogram of percentage of time spent in the target quadrant as measured during the probe test on day 5 (*p=0.05). Individual animal data are shown in gray circles. FIG. 1D: Histogram depicting comparison of the number of platform crossings during probe trial on day 5 (*p=0.05). Individual animal data are shown in gray circles. FIGS. 2E-3F: Associative learning test for BTA-EG₄ injected (intraperitoneally) wild-type mice by fear conditioning. FIG. 2D: Histogram depicting performance of mice treated with BTA-EG₄ (30 mg/kg) daily for 2 weeks before training during the contextual memory test (n=8/group; *p=0.05). Individual animal data are shown in gray circles. FIG. 2F: Histogram depicting that mice were re-exposed to the cue component in a novel context after 24 h, and their behavior was monitored. Mice treated with BTA-EG₄ exhibited significantly enhanced freezing in response to the cue component (n=8/group; *p=0.05). Individual animal data are shown in gray circles. Control (“CTRL”); BTA-EG₄ (“BTA”).

FIGS. 3A-3K. BTA-EG₄ does not enhance LTP in CA1. FIGS. 3A-3F: SC inputs to CA1. FIG. 3A: No significant difference in input-output function. Left, field potential (FP) slope plotted against stimulation intensity. CTRL (solid diamond); n=15 slices, six mice; BTA (BTA-EG₄, triangles): n=18 slices, six mice. Right, FP slope normalized to fiber volley (FV). Top left: Schematic of recording. Top center: Representative control FP traces. Top right: Representative BTA-EG₄ traces. FIG. 3B: No change in presynaptic function. Top panels: Representative control (left) or BTA-EG₄ (right) traces at 50 ms interstimulus interval (ISI). Bottom panel: paired pulse facilitation (PPF) ratio at different ISI. FIGS. 3C-3D: No difference in the magnitude of LTP induced by 1× TBS (CTRL: n=9 slices, 6 mice, BTA: n=10 slices, 5 mice, t test: p=0.359; FIG. 3C) and a reduction with 4× TBS (CTRL: n=8 slices, n=4 mice, BTA: n=11 slices, 6 mice, t test: p=0.01; FIG. 3D) in the BTA-treated group. FIGS. 3C-3D (top panels): Representative traces (baseline: thin line, post-LTP: thick line). FIGS. 3E-3F: Comparison of response integration during 1× TBS (FIG. 3D) and 4× TBS (FIG. 3F). FIGS. 3E-3F (top panels): representative traces. *p_{—}0.05. FIGS. 3G-3K: TA inputs to CA1. FIG. 3G: Isolation of TA inputs by stimulating the stratum lucidum-moleculare (SLM). Left, Schematics of recording. Right, Representative FP traces following stimulation of SC inputs by an electrode placed in stratum radiatum (SR) or SLM when recording from SR or SLM. FIG. 3H: A reduction in input-output function with stimulation intensity (left), which is normalized when corrected for presynaptic recruitment of axons (right) (CTRL: n=24 slices, n=5 mice, BTA: n=23 slices, n=5 mice). Top, Representative traces. FIG. 3I: Normal PPF ratio (CTRL: n=25 slices, n=5 mice, BTA: n=22 slices, n=5 mice). Top, Representative traces taken at 50 ms ISI. FIG. 3J: Reduced TA-LTP (CTRL: 120±2.1% at 1 h post-LTP, n=11 slices, n=5 mice; BTA: 109±1.2%, n=10 slices, n=4 mice; t test, p=0.01). Top, Representative traces. FIG. 3K: Histogram depicting normal summation of responses during TA-LTP induction protocol. Top, Representative traces.

FIGS. 4A-4J. BTA-EG₄ promotes spinogenesis in vivo. FIG. 4A: Representative Golgi-impregnated widefield view of the hippocampus (5× magnification). FIG. 4B: Representative apical oblique (AO) and basal shaft (BS) dendrites from hippocampal CA1 neurons from mice treated with control and BTA-EG₄ (30 mg/kg) as indicated. FIG. 4C: Left histogram columns: Spine density in hippocampal AO dendrites (n=5; **p<0.01). Center histogram columns: Spine density in hippocampal BS dendrites (n=5; **p<0.01). Right histogram columns: Total averaged spine density in hippocampal dendrites (n=5; **p<0.01). FIG. 4D: A representative Golgi impregnated neuron from cortical layers II/III. FIG. 4E: Representative AO and BS dendrites from pyramidal neurons of mice treated with control and BTA-EG₄ (30 mg/kg) as indicated. FIG. 4F: Left histogram columns: Average dendritic spine density for cortical AO dendrites (n=5, ***p<0.001). Center histogram columns: Average dendritic spine density for cortical BS dendrites (n=5, **p<0.01). Right histogram columns: Total average dendritic spine density (n=5, **p<0.01). FIGS. 4G-4H: The cumulative distribution percentage of spine head width FIG. 4G) and spine length (FIG. 4H) in cortical layers II/III in mice treated with BTA-EG₄ (30 mg/kg) (Kolmogorov-Smirnov test). FIGS. 4I-4J: The cumulative distribution percentage of spine head width and spine length in the hippocampus CA1 in mice treated with BTA-EG₄ (30 mg/kg). Scale bar=0.2 mm. Control (“C”); BTA-EG₄ (“B”); cortex (“CTX”); hippocampus (“HPC”).

FIGS. 5A-5D: BTA-EG₄ requires APP to increase spine density. FIG. 5A: Primary hippocampal neurons were transfected with GFP and PLL (upper panels) or GFP and APP shRNA (lower panels), treated with control (left panels) or BTA-EG₄ (5 μM) (right panels), and spine density was measured. FIG. 5B: Histogram depicting quantification of data from FIG. 5A (n=15). FIG. 5D: Representative images of AO and BS dendrites from APP knockout mice treated with control or BTA-EG₄. APP knockout mice were injected with control or BTA-EG₄ for 2 weeks, and Golgi staining was conducted. FIG. 5D: Histogram depicting quantification of data from FIG. 5C (n=5/group). Control (“C”); BTA-EG₄ (“B”).

FIGS. 6A-6L. BTA-EG₄ increases the number of functional synapses without altering synaptic strength. FIGS. 6A-6B: Cultured hippocampal neurons (DIV 18) were treated with BTA-EG₄ (5 μM) or control for 24 h and stained for synaptophysin (FIG. 6A, right panels) and PSD-95 (FIG. 6B, right panels). Neurons and dendrites were visualized by transfection of GFP (FIGS. 6A-6B, left panels). FIG. 6C: Histogram depicting quantification of average puncta number of synaptophysin and PSD-95 per 20 μm length of dendrite (n=10, *p=0.05, **p=0.01). FIG. 6D: BTA-EG₄ (30 mg/kg, i.p.)-treated mice showed significantly increased mEPSC frequency in CA1 pyramidal neurons as shown in histogram of comparison of average mEPSC frequency (CTRL: n=7 cells, 5 mice; BTA: n=8 cells, 5 mice; *t test: p=0.05). Values for individual cells are shown in gray circles. FIGS. 6E-6F: Figures are representative three consecutive mEPSC traces (1 s each) taken from cells of CTRL (FIG. 6E) and BTA (FIG. 6F) cases. FIG. 6G: Figure depicts no significant change in average mEPSC amplitude (n, the same as in FIG. 6D). Values for individual cells are shown in gray circles. Center and rights panels: Average mEPSC trace from control and BTA-EG₄ cases, respectively. FIG. 6H: Histogram (left) depicting no change in the ratio of AMPA/NMDA-mediated synaptic responses. Values for individual cells are shown in gray circles. Center and right panels depict overlap of AMPAR-mediated EPSC measured at −70 mV and NMDAR-mediated EPSC measured at +40 mV for control (center panel) and BTA-EG₄ (right panel) cases. Dotted line shows where NMDAR responses were measured. FIGS. 6I-6L: Figures depict no change in the total and cell surface levels of AMPAR (FIGS. 6I-6J) and NMDAR (FIGS. 6K-6L) subunits in microdissected CA1 slices obtained from control and BTA (30 mg/kg, i.p.)-treated mice. Left, Representative immunoblots. The blots were reprobed for β-actin, which did not show significant difference in signal between control (“C”) and BTA-EG₄-treated (“B”) groups (p=0.269). Right, Average data of glutamate receptor signal normalized to average control (CTRL: n=8 mice, BTA: n=8 mice, t test: p>1). There was no significant difference when the signal for each glutamate receptor antibody was normalized to the actin signal for each total homogenate sample (GluA1/β-actin ratio: CTRL=0.96±0.10, BTA=1.06±0.12,p=0.5; GluA2/β-actin ratio: CTRL=0.98±0.09, BTA=1.27±0.14,p=0.1; GluN1/β-actin ratio: CTRL=0.98±0.12, BTA=1.27±0.05,p=0.1; GluN2A/β-actin ratio: CTRL=1.03±0.08, BTA=1.04±0.14, p=1.0; GluN2B/β-actin ratio: CTRL=1.02±0.11, BTA=1.33±0.14,p=0.1). C, Control; B, BTA-EG₄.

FIGS. 7A-7L. BTA-EG₄ increases dendritic spine density through Ras signaling. FIG. 7A: Cultured hippocampal neurons (DIV18) were treated with BTA-EG₄ (5 μM) or control for 24 h and stained for RasGRF1. FIG. 7B: Pulldown of active Ras in primary cortical neurons using GST-Raf1-RBD (n=2). FIG. 7C: Pulldown of active Ras in brain lysates from wild-type mice intraperitoneally injected with BTA-EG₄ (30 mg/kg) daily for 2 weeks, using GST-Raf1-RBD (n=3). FIGS. 7D-7G: Cultured hippocampal neurons (DIV18) were treated with BTA-EG₄ (5 μM) or control for 24 h, and stained for p-ERK (FIG. 7D), ERK (FIG. 7E), p-CREB (FIG. 7F), and CREB (FIG. 7G). FIG. 7H: Histogram of quantification and comparison, in order left to right, of RasGRF1 (FIG. 7A, A, n=15), p-ERK (FIG. 7D, n=21), ERK (FIG. 7E, n=30), p-CREB (FIG. 7F, n=15), and CREB (FIG. 7G, n=15) intensities (**p<0.01; *p<0.05). FIG. 7I: Primary hippocampal neurons were transfected with GFP and PLL (top) or GFP and RasGRF1 shRNA (bottom) and treated with BTA-EG₄ (5 μM) or control. FIG. 7J: Histogram of quantification of dendritic spine density from FIG. 7I (**p<0.01; ***p<0.001). FIG. 7K: Primary hippocampal neurons were transfected with GFP and vector, GFP and Ras-WT, and GFP and RasN17, and were treated with BTA-EG₄ (5 μM) or control. FIG. 7L: Histogram of quantification of dendritic spine density from FIG. 7K (n=23, *p<0.05; **p<0.01; ***p<0.001). C, Control; B, BTA-EG₄.

FIGS. 8A-8I: APP interacts with RasGRF1 and regulates Ras signaling proteins. FIG. 8A: Brain lysates from wild-type mice were immunoprecipitated (IP) with IgG or APP, and probed with RasGRF1. FIG. 8B: Brain lysates from wild-type mice were immunoprecipitated with IgG or RasGRF1, and probed with APP. FIGS. 8C-8D: Pulldown of active Ras from wild-type mice (FIGS. 8C-8D, n=5), APP transgenic (TG) mice (1 month old, FIG. 8C) or APP knock-out mice (FIG. 8D) using GST-Raf1-RBD. FIG. 8E: Histogram of quantification of data from FIGS. 8C-8D. FIGS. 8F-8H: Primary hippocampal neurons were transfected with GFP and PLL or GFP and APP shRNA, then immunostained with RasGRF1 (FIG. 8F, n=25), p-ERK (FIG. 8G, n=25), and p-CREB (FIG. 8H, n=25). FIG. 8I: Histogram of quantification of data shown in FIGS. 8F-8H (***p<0.001).

FIGS. 9A-9G. BTA-EG₄ requires APP to alter Ras signaling. FIG. 9A: Primary hippocampal neurons were transfected with GFP and APP shRNA (top) or GFP and APP (bottom), treated with control or BTA-EG₄ (5 μM), then immunostained with RasGRF1. FIG. 9b: Histogram of quantification of Ras GRF1 levels from FIG. 9A (n=20, ***p<0.001). FIG. 9c: Pulldown of active Ras from APP knock-out mice using GST-Raf1-RBD (Ras binding domain) following injection of control or BTA-EG₄ (30 mg/kg, i.p.) for 2 weeks. There was no significant difference in the amount of active Ras between APP KO mice treated with control or BTA-EG₄ [CTRL (C)=100±1.22%, BTA (B)=03.2±1.26%, n=5]. FIG. 9E: Primary hippocampal neurons were transfected with GFP and APP shRNA (top) or GFP and APP (bottom), treated with control or BTA-EG₄ (5 μM), then immunostained with p-ERK. FIG. 9E: Histogram of quantification of p-ERK (n=20, ***p<0.001). FIG. 9F: Primary hippocampal neurons were transfected with GFP and APP shRNA (top) or GFP and APP (bottom), treated with control or BTA-EG₄ (5 μM), then immunostained with p-CREB. FIG. 9G: Histogram of quantification of p-CREB (n=20, ***p<0.001).

FIGS. 10A-10J. BTA-EG₄ increases dendritic spine density in 3× TgAD mice. FIGS. 10A-10E: Representative Golgi-stained dendritic segments of cortical layer II/III pyramidal neurons from 6-10 months of age (FIG. 10A) or 13-16 months of age (FIG. 10B) 3× Tg AD mice treated with BTA-EG₄ (“B”) or vehicle (“C”) control. Histogram of quantification of averaged spine densities on apical oblique (AO) (FIG. 10C), basal (BS) (FIG. 10D), and total (AO+BS) (FIG. 10E) dendrites (n=4-5 brains/group; **p<0.01, ***p<0.001). (FIGS. 10E-10J) Representative Golgi-stained dendritic segments of cortical layer II/III pyramidal neurons from 6 to 10 months of age (FIG. 10F) or 13-16 months of age (FIG. 10G) 3× Tg AD mice treated with BTA-EG₄ or vehicle control. Histograms of quantification of averaged spine densities on apical oblique (AO, FIG. 10H), basal (BS, FIG. 10I), and total (AO+BS, FIG. 10J) dendrites (n=3 brains/group; **p<0.01, ***p<0.001).

FIGS. 11A-11L. BTA-EG₄ alters dendritic spine morphology in 6-10 month-old, but not 13-16 month old, 3× Tg AD mice. FIGS. 11A-11D: Dendritic spine morphology is depicted as a cumulative distribution plot of spine head width (FIGS. 11A, 11C) and spine length (FIG. 11B, 11D) in cortical layers II/III (FIGS. 11A-11B) and hippocampal region CA1 (FIGS. 11C-11D) in 6-10 month old mice treated with BTA-EG₄ (Kolmogorov-Smirnov test, n=4-5 brains/group; *p<0.05). FIGS. 11E-11H: Dendritic spine morphology is depicted as a cumulative distribution plot of spine head width (FIGS. 11E, 11G) and spine length (FIGS. 11F, 11H) in cortical layers II/III (FIGS. 11E-11F) and hippocampal region CA1 (FIGS. 11G-11H) at 13-16 month old mice treated with BTA-EG₄ (Kolmogorov-Smirnov test, n=3 brains/group). FIGS. 11I-11L: Histogram summary of the average width of dendritic spines in the cortex (FIG. 11I) and hippocampal CA1 region (FIG. 11K) following BTA-EG₄ treatment as a percentage of control levels for each age. Summary of the averaged dendritic spine lengths in the cortex (FIG. 11J) and hippocampal CA3 (FIG. 11L) following BTA-EG₄ (“B”) or control (“C”) treatment for 2 weeks a percentage of control levels for each age. ***p<0.001.

FIGS. 12A-12P. BTA-EG₄ increases Ras activity and RasGRF1 levels in 6-10 month old 3× Tg AD mice. GST-Raft-RBD pull-down of active Ras from brain lysates of cortex and hippocampus from 6-10 month old (FIGS. 12A-12D) and 13-16 month old (FIGS. 12E-12H) 3× Tg AD mice injected with control or BTA-EG₄ (n=2 brains/group); *p<0.05. FIGS. 12I-12L: Western blot and histogram of quantification of RasGRF1 in brain lysates from cortex (FIG. 12I, 12J) and hippocampus (FIG. 12K, 12L) from 6 to 10 month old 3× Tg AD mice i.p. injected with vehicle control (“C”) or BTA-EG₄ (“B”) (n =3-4 brains/group). FIGS. 12M-12P: Western blot and histogram of quantification of RasGRF1 from cortex (FIGS. 12M, 12N) and hippocampus (FIGS. 12O, 12P) from 13 to 16 month old 3× Tg AD mice injected with BTA-EG₄ or vehicle (n=3-4 brains/group). β-Actin is used as a loading control; *p<0.05.

FIGS. 13A-13H. BTA-EG₄ injected mice had increased AMPA receptor subunit GluA2 expression at 6-10 months of age. FIGS. 13A-13D: Western blot of GluA1 and GluA2 levels and histogram of quantification in brain lysates from cortex (FIGS. 13A-13B) and hippocampus (FIGS. 13C13D) of 6-10 month old 3× TgAD mice injected with BTA-EG₄ (“B”) or control vehicle solution (“C”) daily for 2 weeks (n=3-4 brains/group). FIGS. 13E-13H: Western blot of levels of GluA1 and GluA2 and histogram of quantification in brain lysates from cortex (FIGS. 13E-13F) and hippocampus (FIGS. 13G-13H) of 13-16 month old 3× Tg AD mice i.p. injected with BTA-EG₄ (“B”) or control (“C”) (n=3-4 brains/group).

FIGS. 14A-14O. BTA-EG₄ improves cognitive performance of 3× TgAD mice. Spatial learning was tested by Morris Water Maze in 3× Tg mice aged 2-3 months (n=7/group), 6-10 months (n=10/group), and 13-16 months (n=7/group). Escape latencies during the 4-day training phase (FIGS. 14A, 14E, 14I), histogram of results swim speed velocity during the training trials (FIGS. 14B, 14F, 14J), histogram of percent time spent in the target quadrant measured during the probe test on day 5 (FIGS. 14C, 14G, 14K), and histogram of the number of platform crossings during probe trial on day 5 (FIGS. 14D, 14H, 14L) were compared between vehicle (CTRL) and BTA-EG₄ (BTA) treated 2-3 month old (FIGS. 14A-14D), 6-10 month old (FIGS. 14E-14H), and 13-16 month old (FIGS. 14I-14L) 3× Tg AD mice (*p<0.05, **p<0.01). Aβ ELISA was conducted to compare Aβ levels in 2-3 month old (FIG. 14M, n=7/group), 6-10 month old (FIG. 14N, n=9/group), and 13-16 month old (FIG. 14O, n=4-5/group) 3× Tg AD mice injected with BTA-EG₄ or vehicle daily for 2 weeks.

FIGS. 15A-15H. FIG. 15A: Representative Golgi-stained dendritic segments of cortical layer II/III pyramidal neurons from 2-3 months of age 3× Tg AD mice (FIG. 10E) or from hippocampus (FIG. 15E) treated with BTA-EG₄ (“BTA”) or vehicle (“CTRL”) control. FIGS. 15B-15D, 15F-15H: Histograms depict quantification of averaged spine densities on apical oblique (AO) (FIGS. 15B, 15F), basal (BS) (FIGS. 15C, 15G), and total (AO+BS) (FIGS. 15D, 15H). See FIGS. 10A-10J. n=3 brains/group.

FIGS. 16A-16B. FIG. 16A: Figure depicts histogram of dendritic spin density in cortex for 3× Tg AD mice as a function of age (ordered pairs left to right: 2-3 mo, 6-10 mo, 13-16 mo) and as a function of treatment (control “C”, BTA-EG₄ “B”). FIG. 16B: Figure depicts histogram of dendritic spin density in hippocampus for 3× Tg AD mice as a function of age (ordered pairs left to right: 2-3 mo, 6-10 mo, 13-16 mo) and as a function of treatment (control “C”, BTA-EG₄ “B”). See FIGS. 10A-10J. n=3 brains/group.

FIGS. 17A-17H. FIGS. 17A-17F: Assay for p-ERK, ERK and β-actin in 6-10 month old 3× Tg AD mice (FIGS. 17A, 17D) in cortex (FIGS. 17A-17C) and hippocampus (FIGS. 17D-17F) under control (“C”) conditions and after injection with 30 mg/kg BTA-EG₄ (“B”) for two weeks prior to assay. FIG. 17G: Assay depicting that BTA-EG₄ administration in 6-10 month old 3× Tg AD mice did not alter the levels of p-Elk, which is a downstream target of phosphorylated ERK. FIG. 17H: Assay depicting that BTA-EG₄ administration in 13-16 month old 3× Tg AD mice did not alter the levels of p-Elk.

### DETAILED DESCRIPTION OF THE INVENTION

### I. DEFINITIONS

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from:
      oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In embodiments, at least one or all of these groups are substituted with at least one size-limited substituent group. In embodiments, at least one or all of these groups are substituted with at least one lower substituent group.

In embodiments of the compounds herein, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating,” and conjugations thereof, include prevention of an injury, pathology, condition, or disease.

An “effective amount” is an amount sufficient to accomplish a stated purpose (e.g.

achieve the effect for which it is administered, treat a disease, reduce one or more symptoms of a disease or condition, and the like). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Subject,” “patient,” “subject in need thereof” and the like refer to a living organism suffering from or prone to a disease or condition that can be treated by administration of a compound or pharmaceutical composition as provided herein. Non-limiting examples include humans, other mammals, bovines, rats, mice, dogs, monkeys, goat, sheep, cows, deer, and other non-mammalian animals. In embodiments, a subject is human.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, dimethyl sulfoxide (DMSO), NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, polyethylene glycol, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “administering” means oral administration, administration as an inhaled aerosol or as an inhaled dry powder, suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compound of the invention can be administered alone or can be coadministered to the patient. Coadministration is meant to include simultaneous or sequential administration of the compound individually or in combination (more than one compound or agent). The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Pharmaceutical compositions provided by the present invention include compositions wherein the active ingredient (e.g. compounds described herein, including embodiments or examples) is contained in a therapeutically effective amount, i.e., in an amount effective to achieve its intended purpose. The actual amount effective for a particular application will depend, inter alia, on the condition being treated. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, tautomers, geometric isomers, and individual isomers are encompassed within the scope of the present invention. The compounds of the present invention do not include those that are known in the art to be too unstable to synthesize and/or isolate.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

### II. METHODS OF TREATMENT

In a first aspect, there is provided a method for improving memory or learning in a subject in need thereof, the method including administering to the subject an effective amount of a compound of Formula (I):

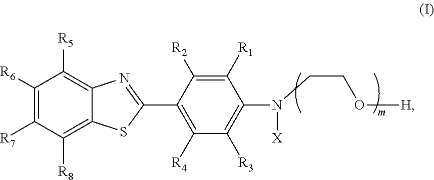

wherein R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyland trifluoromethyl; m is a integer in the range 1-20; and X is hydrogen, methyl, or ethyl. The improving is relative to the absence of administration of the compound.

The term “memory” and the like refer, in the usual and customary sense, to the processes by which information is encoded, stored and retrieved by a subject. The terms “encode,” “register” and the like in the context of memory refer, in the usual and customary sense, to receiving, processing and combining information impinging on the senses as chemical or physical stimuli. The term “stored” and the like in this context refer, in the usual and customary sense, to the creation of a record of the encoded information. The terms “retrieve,” “recall” and the like in this context refer, in the usual and customary sense, to calling back the stored information. Retrieval can be in response to a cue, as known in the art.

In embodiments, the memory may be recognition memory or recall memory. In this context, “recognition memory” refers to recollection of a previously encountered stimulus. The stimulus can be e.g., a word, a scene, a sound, a smell or the like, as known in the art. A broader class of memory is “recall memory” which entails retrieval of previously learned information, e.g., a series of actions, list of words or number, or the like, which a subject has encountered previously. Methods for accessing the level of memory encoding, storage and retrieval demonstrated by a subject are well known in the art, including methods disclosed herein.

The term “learning” and the like refer, in the usual and customary sense, to the acquisition of knowledge, behaviors, skills, values or preferences, or modifying and reinforcing what has been previously learned. Without wishing to be bound by any theory, it is believed that synaptic plasticity is correlated with learning. See e.g., Kandel, 2001; VanGuilder et al., 2011. The term “synaptic plasticity” and the like refer, in the usual and customary sense, to the ability of synapses to strength or weaken over time. Mechanisms of synaptic plasticity are known in the art. In particular, without wishing to be bound by any theory, it is believed that long-lasting changes in the efficacy of synaptic connections can implicate the making and breaking of synaptic contacts; i.e., “long-term potentiation (LTP).” It is further believed that synaptic plasticity can result from modulation (i.e., increase or decrease) in the density of receptors, e.g., on post-synaptic membranes. The term “spinogenesis” and the like refer, in the usual and customary sense, to development (e.g. growth and/or maturation) of dendritic spines in neurons. In embodiments, the compounds provided herein promote spinogenesis without affecting spine morphology. The promotion is relative to the absence of administration of the compound.

In embodiments, compounds useful in the methods provided herein have the structure of Formula (I) defined above. In embodiments, compounds useful in the methods provided herein have the structure of Formula (Ia) (also referred to herein as “BTA-EG₄” or “BTA-EG₄”) :

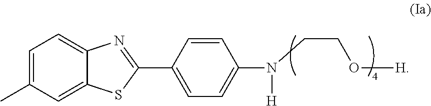

In embodiments of Formula (I) or (Ia), m is an integer in the range 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2. In embodiments, m is 1, 2, 3, 4, 5, 6, 7, 8, 9, 10, 11, 12, 13, 14, 15, 16, 17, 18, 19 or 20. In embodiments, m is 2, 4 or 6. In embodiments, m is 2. In embodiments, m is 3. In embodiments, m is 4. In embodiments, m is 5. In embodiments, m is 6.

In embodiments of Formula (I), R¹, R², R³ and R⁴ are hydrogen. In embodiments, one of R⁵, R⁶, R⁷ and R⁸ is deuterium, tritium, fluoride, chloride, bromide, iodide, hydroxide, amino, methylamino, dimethylamino, trimethylammonium, methyl, ethyl, methoxy, ethoxy, fluoromethyl, or difluoromethyland trifluoromethyl, and the others of R⁵, R⁶, R⁷ and R⁸ are hydrogen. In embodiments, R⁵ is fluoride, chloride, bromide, iodide, hydroxide, amino, methylamino, dimethylamino, trimethylammonium, methyl, ethyl, methoxy, ethoxy, fluoromethyl, or difluoromethyland trifluoromethyl. In embodiments, R⁶ is fluoride, chloride, bromide, iodide, hydroxide, amino, methylamino, dimethylamino, trimethylammonium, methyl, ethyl, methoxy, ethoxy, fluoromethyl, or difluoromethyland trifluoromethyl. In embodiments, R⁷ is fluoride, chloride, bromide, iodide, hydroxide, amino, methylamino, dimethylamino, trimethylammonium, methyl, ethyl, methoxy, ethoxy, fluoromethyl, or difluoromethyland trifluoromethyl. In embodiments, R⁸ is fluoride, chloride, bromide, iodide, hydroxide, amino, methylamino, dimethylamino, trimethylammonium, methyl, ethyl, methoxy, ethoxy, fluoromethyl, or difluoromethyland trifluoromethyl. In embodiments, R⁷ is methyl or ethyl. In embodiments, R⁷ is methyl.

In another aspect, there is provided a method for treating neuronal or cognitive impairment in a subject in need thereof. The method includes administering to the subject an effective amount of a compound of Formula (I), and embodiments thereof (e.g. Formula (Ia)), as disclosed herein. The term “neuronal impairment” and the like refer, in the usual and customary sense, to an atrophy or other decrease in the effective functioning of the neuron. For example, it is known that Alzheimer's Disease (AD) presents with neuronal impairment, especially in cortical neurons, e.g., hippocampal neurons and neurons in proximity to the hippocampus. The terms “cognitive impairment,” “cognitive deficit” and the like refer, in the usual and customary sense, to an impairment or deficit of the cognition process. Typical cognitive deficits include deficits in general intellectual performance, e.g., mental retardation, and deficits in specific cognitive abilities, e.g., learning disorders, dyslexia, and the like. Cognitive deficit may be elicited by injury to the brain, neurological disorder, or mental illness. Without wishing to be bound by any theory, it is believed that dendritic spine loss may be correlated with cognitive impairment. Accordingly, it is further believed that increased dendritic spine density can result in treatment of cognitive impairment and in treatment of neuronal impairment. In embodiments, the compound has the structure of Formula (Ia).

In another aspect, there is provided a method of increasing dendritic spine formation, increasing dendritic spine density or improving dendritic spine morphology in a subject in need thereof. The method includes administering to the subject an effective amount of a compound of Formula (I), and embodiments thereof (e.g. Formula (Ia)), as disclosed herein. The improving and increasing is relative to the absence of administration of the compound. The term “dendritic spine formation” and the like refer, in the usual and customary sense to processes which lead to an increased number of dendritic spines or increased development of dendritic spines. Compounds disclosed herein have been demonstrated to regulate dendritic spine formation. Without wishing to be bound by any theory, it is believe that compounds disclosed herein can increase dendritic spine formation by increasing the activity of Ras-ERK signaling proteins through APP. The term “dendritic spine density” and the like refer, in the usual and customary sense, to the number of dendritic spines per unit area. The term “dendritic spine morphology” and the like refer, in the usual and customary sense, to physical characterization of a dendritic spine (e.g. shape and structure). Improvement of dendritic spine morphology is a change in morphology that results in increased fucntionality. As known in the art and disclosed herein, exemplary methods for such characterization include measurement of the dimensions (i.e., length and width) of dendritic spines. Accordingly, the term “improving dendritic spine morphology” generally refers to an increase in length, width, or both length and width of a dendritic spine.

In embodiments, the method increases dendritic spine formation. In embodiments, the method increases dendritic spine density. In embodiments, the method improves dendritic spine morphology.

In another aspect, there is provided a method of increasing functional synapses in a subject in need thereof. The method includes administering to the subject an effective amount of a compound of Formula (I), and embodiments thereof (e.g. Formula (Ia)), as disclosed herein. The increasing is relative to the absence of administration of the compound. The terms “functional synapses” and like refer, in the usual and customary sense, to synapses which are effective in permitting a neuron to pass an electrical or chemical signal to another cell. In contrast, the term “malfunctional synapses” and the like refer to synapses which lack, either fully or partially, normal physiologic functionality. Methods for determining the number of functional synapses in a system, as well known in the art and disclosed herein, include determination of the frequency of AMPA receptor-mediated mEPSCs.

Further to any aspect disclosed above, in embodiments the subject has Alzheimer's Disease (AD). In embodiments, the subject is suspected of having Alzheimer's Disease. In embodiments, the method improves memory and learning in the subject. In embodiments, the method improves memory in the subject. In embodiments, the method improves learning in the subject.

Further to any embodiment disclosed herein, in one embodiment the subject has low Aβ plaque accumulation in the brain relative to an amount of Aβ plaque accumulation in an Alzheimer's disease standard control. Without wishing to be bound by any theory, it is believed that quantification of Aβ plaque accumulation, and the correlation thereof to the presentation of symptoms of Alzheimer's Disease, does not find consensus within the art. Accordingly, the term “Alzheimer's disease standard control” as used herein refers to a level of Aβ plaque accumulation observed in subjects having a diagnosis of Alzheimer's Disease, as judged by a medical or veterinary practitioner. It is known that Alzheimer's Disease is characterized by severe synapse loss, i.e., severe reduction in functional synapses or severe reduction in the density of synapses. It is further known that Aβ plaque accumulation occurs over time and correlates with the appearance of symptoms of Alzheimer's Disease. Accordingly, the term “low Aβ plaque accumulation” and the like as used herein refer to cases wherein the observed neuronal physiology lacks the levels of Aβ plaque accumulation which characterize Alzheimer's Disease. Moreover, because Alzheimer's Disease is a progressive disease, it is observed that younger subjects typically lack levels of Aβ plaque accumulation which characterize older subjects. Thus, the terms “ages before severe synapse loss,” “synaptic loss seen in early AD,” “before high Aβ plaque load,” “before severe Aβ plaque deposition and synapse loss,” “before Aβ plaque accumulation” and the like are understood as embodiments of the term “Aβ plaque accumulation in the brain” as used herein.

In one embodiment wherein the subject has low Aβ plaque accumulation in the brain relative to an amount of Aβ plaque accumulation in an Alzheimer's disease standard control, the method improves memory. Quantification of memory improvement is available by a variety of methods well known in the art, e.g., as disclosed herein.

Further to any aspect disclosed above, in embodiments the subject is a healthy subject (e.g. the subject does not have AD). In embodiments, the subject does not have a neurological disease. In embodiments, embodiment the subject does not have Alzheimer's Disease. In embodiments, the subject is not suspected of having Alzheimer's Disease. In embodiments, the subject is a juvenile and does have Alzheimer's Disease. In embodiments, the subject is an adult and does have Alzheimer's Disease. In embodiments, the subject is a juvenile and is not suspected of having Alzheimer's Disease. In embodiments, the subject is an adult and is not suspected of having Alzheimer's Disease.

Further to any aspect disclosed herein, in embodiments the compound is administered to the subject for a prolonged period. The terms “prolonged period” and the like refer to 14 days or longer of administration, e.g., daily administration. In embodiments, the compound is administered to the subject daily for 2, 3, 4 weeks, or even longer. In embodiments, the compound is administered to the subject daily for 1, 2, 3, 4 months, or even longer. In embodiments, the compound is administered to the subject daily for 1, 2, 3, 4 years, or even longer. In embodiments, the compound is administered to the subject daily for 1, 2, 3, 4 decades, or even longer. In embodiments, the compound is administered more than once per day, e.g., 2, 3, 4 times per day, or even greater.

Further to any aspect disclosed herein, in embodiments the subject has Fragile-X syndrome (FXS). As known in the art, FXS is a genetic syndrome which has been linked to a variety of disorders, e.g., autism and inherited intellectual disability. The disability can present in a spectrum of values ranging from mild to severe. It is observed that males with FXS begin developing progressively more severe problems, typically starting after age 40, in performing tasks which require working memory. This is especially observed with respect to verbal working memory. Visual-spatial memory is not found to be directedly related to age.

For example, in embodiments the method improves memory or learning in a subject in need thereof, wherein the subject has FXS. In embodiments, the method improves memory in the subject. In embodiments, the method improves learning in the subject. In embodiments, the method treats neuronal or cognitive impairment in the subject. In embodiments, the method treats neuronal impairment in the subject. In embodiments, the method treats cognitive impairment in the subject.

Further to any aspect disclosed herein, in embodiments the subject suffers from autism. As known in the art, autism is a disorder of neural development. Without wishing to be bound by any theory, it is believed that austism affects information processing in the brain by altering how nerves and synapses connect and organize In embodiments, the method improves memory in the subject. In embodiments, the method improves learning in the subject. In embodiments, the method treats neuronal or cognitive impairment in the subject. In embodiments, the method treats neuronal impairment in the subject. In embodiments, the method treats cognitive impairment in the subject.

Further to any aspect disclosed herein, in embodiments the subject suffers from schizophrenia. In embodiments, the method improves memory in the subject. In embodiments, the method improves learning in the subject. In embodiments, the method treats neuronal or cognitive impairment in the subject. In embodiments, the method treats neuronal impairment in the subject. In embodiments, the method treats cognitive impairment in the subject.

Further to any aspect disclosed herein, in embodiments the subject suffers from brain injury. Absent express indication to the contrary, the terms “brain injury” and the like refer to an insult to the brain tissue. Types of brain injury include brain damage (i.e., destruction or degeneration of brain cells), traumatic brain injury (i.e., damage accruing as the result of an external force to the brain), stroke (i.e., a vascular incident which temporarily or permanently damages the brain, e.g., via anoxia), and acquired brain injury (i.e., brain damage not present at birth). In embodiments, the method improves memory in the subject. In embodiments, the method improves learning in the subject. In embodiments, the method treats neuronal or cognitive impairment in the subject. In embodiments, the method treats neuronal impairment in the subject. In embodiments, the method treats cognitive impairment in the subject.

### III. EXAMPLES

The examples provided herein are intended to provide embodiments of the invention described herein and not to limit the scope of the invention.

In the examples provided herein, we investigated the biological effects of BTA-EG₄ on synaptic function in vitro and in vivo. We initially found that BTA-EG₄-injected wild-type mice exhibited increased dendritic spine formation, as well as improved learning and memory. The spinogenic activity of BTA-EG₄ is accompanied by an increase in the number of functional synapses as evidenced by an elevated frequency of AMPA receptor-mediated mEPSCs. Furthermore, BTA-EG₄ acts through amyloid precursor protein (APP) to increase Ras activity as well as downstream Ras signaling, which are necessary for its ability to increase dendritic spine density. These data show that BTA-EG₄ is beneficial as a therapeutic treatment for the neuronal and cognitive dysfunction seen in AD by targeting Ras-dependent spinogenesis.

### Example 1

### A Tetra(Ethylene Glycol) Derivative of Benzothiazole Aniline Enhances Ras-Mediated Spinogenesis

Materials and Methods

Synthesis of 2-(2-(2-(2-hydroxyethoxy)ethoxy)ethoxy)ethyl toluenesulfonate. In a clean, dry 1 L round bottom flask equipped with a stir bar, tetra-ethylene glycol (10.0 g, 51.5 mmol) was dissolved in 500 mL dry dichloromethane (DCM) and stirred at room temperature. After 5 min, potassium iodide (1.71 g, 10.3 mmol), Ag₂O (17.9 g, 77.2 mmol), and p-toluenesulfonyl chloride (10.8 g, 56.6 mmol) were successively added to the reaction flask. The reaction mixture was stirred vigorously for 2 h, filtered through Celite to remove the solids and concentrated in vacuo. The residue was purified via silica column chromatography (100% DCM to 95:5 DCM:CH₃OH) giving 2-(2-(2-(2-hydroxyethoxy)ethoxy)ethoxy)ethyl toluenesulfonate as a colorless oil (13.2 g, 74%). ¹H-NMR (400 MHz, CDCl₃): δ=7.74 (d, 8.0 Hz, 2H), 7.30 (d, 8.0 Hz, 2H), 4.11 (t, 4.8 Hz, 2H), 3.66-3.53 (m, 12H), 2.79 (s, 1H), 2.39 (s, 3H). ¹³C-NMR (100 MHz, CDCl₃); δ=145.04, 133.17, 130.10 (2C), 128.19 (2C), 70.95, 70.79, 70.70, 69.49, 68.88, 21.87. ESI-MS (m/z) calculated for C₁₅H₂₄O₇S [M]+348.1243; found [M+H]+ 348.96, [M+NH₄]⁺ 365.94 and [M+Na]⁺ 371.08.

Synthesis of 2-(2-(2-(2-iodoethoxy)ethoxy)ethoxy)ethanol. 2-(2-(2-(2-hydroxyethoxy)ethoxy)ethoxy)ethyl toluenesulfonate (12.01 g, 34.5 mmol), sodium iodide (20.7 g, 137.9 mmol) and 200 mL dry acetone were combined in a clean, dry round bottom flask and heated to reflux with vigorous stirring. After 12 h the reaction was cooled to room temperature and diluted with 100 mL ethyl acetate. The organic phase was washed with 10% Na₂S₂O₃, (2×10 mL), deionized H₂O (1×20 mL), saturated NaCl (1×20 mL), dried over anhydrous Na₂SO₄, filtered, and concentrated in vacuo giving 2-(2-(2-(2-iodoethoxy)ethoxy)ethoxy)ethanol as a pale yellow oil (5.61 g, 54%). ₁H-NMR (400 MHz, CDCl₃): δ=3.73-3.58 (m, 14H), 3.24 (t, 2H), 2.59 (s, 1H). ¹³C-NMR (100 MHz, CDCl₃); δ=72.70, 72.19, 70.90, 70.76, 70.58, 70.39, 61.94, 3.07.

Synthesis of BTA-EG₄. A microwave reaction tube was charged with 2-(2-(2-(2-iodoethoxy)ethoxy)ethoxy)ethanol (1.47 g, 4.83 mmol), benzothiazole aniline (3.49 g, 14.5 mmol), potassium carbonate (3.34 g, 24.2 mmol) and 20 mL dry THF. The tube was then equipped with a small stir bar, sealed and placed in a microwave reactor. The reaction was heated at 125° C. for 2 h. The reaction was cooled to room temperature and filtered to remove the solids. The solids washed several times with DCM until the filtrate was colorless. The combined organic layers were concentrated in vacuo and purified by column chromatography to give the desired BTA-EG₄ compound as a yellow solid. (1.13 g, 56%). ₁H-NMR (400 MHz, CDCl₃): δ=7.87 (d, 8.8 Hz, 2H), 7.83 (d, 8.4 Hz, 1H) 7.63 (s, 1H) 7.23 (d, 8.4 Hz, 1H), 6.68 (d, 8.8 Hz, 2H), 3.76-3.64 (m, 14H), 3.37 (t, 5.2 Hz, 2H), 2.47 (s, 3H). ¹³C-NMR (100 MHz, CDCl₃); δ=168.03, 152.64, 150.92, 134.87, 134.47, 129.13 (2C), 127.70, 122.88, 122.03, 121.41, 112.82 (2C), 72.86, 70.88, 70.69, 70.43 (2C), 69.64, 61.91, 43.32, 21.70. HR-ESI-MS (m/z) calculated for C₂₂H₂₈N₂O₄SNa [M+Na] 439.1662; found [M+Na]+439.1660.

Cerebrovascular permeability and pharmacokinetic analysis of BTA-EG₄ in wild-type mice. The partitioning of BTA-EG₄ between plasma and brain was studied in male CD-1 mice. The mice were dosed with 10 mg/kg (in 10% DMSO/90% PBS) intraparitoneally (IP, n=2 per time point) and the concentration of BTA-EG₄ in the plasma and brain was measured over time. Blood was collected via cardiac puncture and was pooled in EDTA tubes, centrifuged, and the plasma isolated. The brain was collected from each mouse, snap frozen, and homogenized in 2 mL PBS. The concentration of BTA-EG₄ in the plasma and brain at each time point was determined by LC/MS/MS and the concentrations of BTA-EG₄ in the plasma and brain were plotted as a function of time. Pharmacokinetic parameters for the plasma and brain profile of BTA-EG₄ were also calculated: half-life for BTA-EG₄ in the plasma and brain (t_{1/2}), the maximum concentration (Cₘₐₓ) of BTA-EG₄ in the plasma and brain, the area under the concentration-time curve (AUC), the brain-to-plasma ratio (BB), and the logarithmic brain-to-plasma ratio (Log BB).

Cell lines. COS 7 cells (Lombardi Co-Resources Cancer Center, Georgetown University) were maintained in Opti-MEMO (Invitrogen) with 10% fetal bovine serum (FBS, Life Technologies, Inc.) in a 5% CO₂ incubator. The cells were transiently transfected with 0.5-1 μg of plasmid in FuGENE® 6 (Roche) according to the manufacturer's protocol and cultured 24 hr in DMEM containing 10% FBS. For co-transfections, cells were similarly transfected with 0.5-1 μg of each plasmid in FUGENE® 6 (Roche) and cultured 24 hr in DMEM with 10% FBS.

Primary neuron culture and immunostaining. Primary hippocampal neurons from E19 Sprague-Dawley rats were cultured at 150 cells/mm² as previously described (Pak et al., 2001). Neurons were transfected using Lipofectamine™ 2000 (Invitrogen, Carlsbad, Calif., USA) or calcium phosphate precipitation with GFP+PLL, GFP+APP shRNA, or GFP+APP and treated with BTA-EG₄. The following antibodies were used: mouse anti-GFP (Novus Biologicals, 9F9.F9), rabbit anti-GFP (Invitrogen, A11122), rabbit anti-GluR1 (Calbiochem, PC246), mouse anti-GluR2 (BD Pharmagen, 556341), mouse anti-postsynaptic density (PSD)-95 (NeuroMabs, Davis, Calif., USA), mouse anti-Synaptophysin (Sigma Aldrich, s5768), rabbit APP N-terminal (Sigma-Aldrich, A8967), anti-Ras (BD Biosciences, 610001), anti-RasGRF1 (Santa Cruz, C-20; BD Biosciences, 610149), anti-ERK1/2 (Cell Signaling, 4695), anti-p-ERK1/2 (Invitrogen, 36880), anti-CREB (Cell Signaling, D76D11), anti-p-CREB (Millipore, 06-519), and mouse anti-c-Myc (Novus Biologicals, 9E10). Cultured hippocampal neuron images were acquired by LSM 510 laser scanning confocal microscope (Zeiss). Confocal zseries image stacks encompassing entire dendrite segments were analyzed using MetaMorph software (Universal Imaging Corporation, Downington, Pa., USA).

GST-pull-down assay. To measure levels of active Ras, brain lysate homogenized with Ral buffer (25 mM Tris-HCl, pH 7.4, 250 mM NaCl, 0.5% NP40, 1.25 mM MgCl₂, and 5% glycerol) from APP transgenic, APP knockout, or wild-type mice was incubated with GST-Raf-RBD purified protein coupled with GLUTATHIONE SEPHAROSE™ (Amersham) overnight at 4° C. After 24 hours, pellets were washed with Ral buffer and western blotting was conducted with anti-Ras.

Cell surface biotinylation. COS7 cells were transiently transfected with APP for 24 hr in OPTI-MEM® containing 10% fetal bovine serum, and then treated with BTA-EG₄ or control for 24 hr. After 24 hr, surface proteins were biotin labeled, immobilized with NeutrAvidin™ Gel and incubated 1 h with SDS-PAGE sample buffer, including 50 mM DTT, as described previously (Minami et al., 2010). Eluates were analyzed for APP by immunoblotting.

Live cell surface immunostaining. Immunostaining of surface APP in hippocampal neurons was performed as described (Hoe et al., 2009). Briefly, live neurons were incubated with APP antibodies (10 μg/mL in conditioned medium) for 10 min, and then briefly fixed in 4% paraformaldehyde (non-permeabilizing conditions). Surface-labeled APP was detected with ALEXA FLUORO-555 secondary antibodies. Cells were then permeabilized in methanol (−20° C., 90 s), and incubated with anti-GFP antibody to identify transfected neurons.

Animals. Wild-type C57BL/6J and APP knockout mice (B6.12957-APP_{tm1Dbo}/J) were obtained from Jackson Laboratories (Bar Harbor, Me., USA). CD1 mice were obtained from Vivasource. All animal experiments were approved by the Institutional Animal Care and Use Committees at Georgetown University and Johns Hopkins University. All animals were maintained according to protocols approved by the Animal Welfare and Use Committee at both institutes.

Golgi staining and morphological analysis of dendritic spines. To analyze dendritic spine density and morphology in brain, FD RAPID GOLGISTAIN KIT™ (FD NeuroTechnologies, Ellicott City, Md., USA) was used. Dissected mouse brains were immersed in Solution A and B for 2 weeks in dark conditions at room temperature and transferred into Solution C for 24 h at 4° C. Brains were sliced using a VT1000S Vibratome (Leica, Bannockburn, Ill., USA) at 150 μm thickness. Dendritic images were acquired by Axioplan 2 (Zeiss, Oberkochen, Germany) under brightfield microscopy. Spine width, length, and linear density of cortical layers II/III and CA1 of hippocampus were measured using Scion image software (Scion Corporation, Frederick, Md., USA). Images were coded, and dendritic spines counted in a blinded manner. Spines from 0.2 to 2 μm in length were included for analysis. All morphological analysis was done blind to experimental conditions.

Aβ ELISA. Mouse brains were homogenized in tissue homogenization buffer containing 250 mM sucrose, 20 mM Tris base, protease, and phosphatase inhibitors. To measure soluble Aβ, DEA extraction was performed. Crude 10% homogenate was mixed with an equal volume of 0.4% diethylamine (DEA), sonicated, and ultracentrifuged for 1 hr at 100,000×g. The supernatant was collected and neutralized with 10% 0.5 M Tris, pH 6.8. Sensitive and specific ELISAs to rodent Aβ1-40 was purchased from IBL Transatlantic (Toronto, Canada) and conducted per the manufacturer's protocol.

Morris water maze. To examine the effects of BTA-EG₄ on cognitive performance, we injected wild-type (B6) mice with BTA-EG₄ daily for one week, and then began behavior testing. We continued daily injections for another week, for a total treatment course of two weeks. The Morris water maze task included training mice to locate a submerged, hidden platform using extramaze visuospatial cues. This system consists of a large, white circular pool with a Plexiglas platform painted white and submerged below the surface of the water, made opaque by the addition of white non-toxic paint. During training, the platform was hidden 14 inches from the side wall in one quadrant of the maze. The animals were gently placed at random into one of the four quadrants, separated by 90°, and facing the wall. The time required (latency) to locate the hidden platform was recorded by a blinded observer and tracked using TOPSCAN, and was limited to 90 sec. Animals failing to find the platform within 90 sec were assisted to the platform. Animals were allowed to remain on the platform for 15 sec on the first trial and 10 sec on subsequent trials. 24 hrs after the final learning trial, a probe trial of 90 sec was given. We recorded the percentage of time spent in the quadrant where the platform was previously located. As a control experiment, we tested motor impairment or visual discriminative ability. The animals were required to locate a clearly visible black platform (placed in a different location), raised above the water surface, at least 12 hrs after the last trial.

Fear conditioning. Before initiating fear-conditioning tests, wild-type mice were injected daily with BTA-EG₄ or vehicle for 2 weeks (n=8/group). Mice were subsequently trained by exposure to a conditioning box (context) for 3 min before administering a tone (18 s) and footshock (2 s), which were repeated twice at 1 min intervals. We performed the contextual test 24 h after training by re-exposing the mice to the conditioning context. A measurement of freezing behavior was recorded every minute for 3 min inside of the conditioning box. The cued test was conducted 3 h following the contextual test. Following a 3 min reexposure to the conditioning box, a tone (18 s) was administered and freezing behavior was measured for 1 min.

Electrophysiology. Transverse [for Schaffer collateral (SC) input studies] or horizontal [for temporammonic (TA) input studies] hippocampal slices (400-μm thick) were prepared from adult mice. Slices were made using ice-cold dissection buffer (in mM: 212.7 sucrose, 2.6KCl, 1.23NaH₂PO₄, 10 dextrose, 3MgCl₂ and 1CaCl₂; 5% CO₂/95% O₂), and recordings were done in a submersion-type chamber perfused with artificial cerebrospinal fluid (ACSF, in mM: 124NaCl, 5KCl, 1.25NaH₂PO₄, 26NaHCO₃, 10 dextrose, 1.5MgCl₂, and 2.5CaCl₂; 5% CO₂/95% O₂, 29.5-30.5° C., 2 ml/min). For field potential recordings, synaptic responses were evoked using 0.2 ms duration pulses delivered through a bipolar glass stimulating electrode at 0.0333 Hz. A train of TBS consisted of a burst of 4 pulses at 100 Hz repeated 10 times at 5 Hz. For 4× TBS, 4 trains of TBS were given at 10 sec inter-train-intervals. For whole cell recordings, slices were transferred to a submersion-type recording chamber mounted on a fixed stage of an upright microscope (E600 FN; Nikon, Tokyo, Japan) with IR oblique illumination. AMPA receptor (AMPAR)-mediated miniature excitatory postsynaptic currents (mEPSCs) were pharmacologically isolated by adding 1 μM tetrodotoxin, 20 μM bicuculline, and 100 μM D,L-2-amino-5-phosphonopentanoic acid to ACSF (30±1° C., saturated with 95% O₂-5% CO₂), which was continually perfused at a rate of 2 ml/min. Target cells in CA1 were identified by the pyramid-shaped soma. These neurons were patched using a whole cell patch pipette (tip resistance 3-5 MΩ), which was filled with internal solution (in mM: 120 Cs-methanesulfonate, 5MgCl₂, 8NaCl, 1 EGTA, 10 HEPES, 2 Mg.ATP, 0.5Na₃GTP and 1 QX-314; pH 7.3, 280-290 mOsm). Recording was initiated 2-3 min after break-in, and each cell was recorded for 10-15 min to collect enough mEPSCs for analysis. The Axon patch-clamp amplifier 700B (Molecular Devices, Union City, Calif.) was used for voltage-clamp recordings. Cells were held at −70 mV, and the recorded mEPSC data were digitized at 10 kHz with a data acquisition board (National Instruments, Austin, Tex.) and acquired through custom-made programs using the Igor Pro software (WaveMetrics, Lake Oswego, Oreg.). The MiniAnalysis program (Synaptosoft, Decatur, Ga.) was used to analyze the acquired mEPSCs. The threshold for detecting mEPSCs was set at three times the root mean square (RMS) noise. There was no significant difference in the RMS noise across the groups [BTA-EG₄ (30 mg/kg)=1.6±0.09 (n=7), Control=1.6±0.05 (n=8); t-test, P>0.65]. Recordings were excluded from analysis if the RMS noise was <2, the series resistance was <25 MΩ, and input resistance was >100 MΩ. To minimize the impact of dendritic filtering, we adopted the standard approach of excluding mEPSCs with rise time >3 ms, as well as cells showing a negative correlation between mEPSC amplitude and rise time (Rall, 1969). Only about 4% of the total recorded cells (at most 1 cell per experimental group) were excluded due to negative correlation between mEPSC rise and amplitude. One hundred consecutive mEPSCs that met the rise time criteria were analyzed from each cell. AMPAR- and NMDAR-mediated currents were measured at −70 and +40 mV, respectively. AMPAR mediated EPSC amplitudes were measured at the peak of the current at −70 mV. NMDAR mediated EPSC amplitudes were measured 100 ms after the stimulation artifact. Data are means±SE. Student's t-test was used for two-group comparisons. For all statistical tests, P<0.05 was considered statistically significant.

Slice surface biotinylation. Hippocampal slices (400 μm thick) were prepared as described above. CA3 and DG regions were cut away after the slice preparation to isolate CA1. After 30 min recovery at room temperature, the slices were transferred to 30° C. for additional 30 min recovery. The slices were then transferred to ice-cold ACSF for 10 min, and subsequently to ice-cold ACSF containing 1 mg/ml biotin (EZ-Link Sulfo-NHS-SS-Biotin, Pierce) saturated with 5% CO₂/95% O₂ for 15 min. The slices were then washed in tris-buffered saline (TBS: 50 mM Tris, 0.9% NaCl, pH 7.4) containing 100 mM glycine 5 times for 1 min each before homogenized in ice-cold 0.2% SDS/1% Triton X-100 IPB (20 mM Na₃PO₄, 150 mM NaCl, 10 mM EDTA, 10 mM EGTA, 10 mM Na₄P₂O₇, 50 mM NaF, and 1 mM Na₃VO₄, pH 7.4; with 1 mM okadaic acid and 10 KIU/ml aprotinin) by 30 gentle strokes using glass-Teflon tissue homogenizers (Pyrex). The homogenates were centrifuged for 10 min at 13,200×g, 4° C. Protein concentration of the supernatant was normalized to 0.6-1.5 mg/ml. Some of the supernatants were saved as inputs, and the remaining supernatant was mixed with neutravidin slurry [1:1 in 1% Triton X-100 IPB (TX-IPB)] and rotated overnight at 4° C. The neutravidin beads were isolated by brief centrifugation at 1,000×g, and washed 3×1% TX-IPB, 3×1% TX-IPB+500 mM NaCl, followed by 2×1% TX-IPB. The biotinylated surface proteins were then eluded from the neutravidin beads by rotating at room temperature for 15 min in gel sample buffer with 2 mM DTT. The input (total homogenate) and biotinylated samples (surface fraction) were run on separate gels, and processed for immunoblot analysis using GluA1 (sc-55509, Santa Cruz), GluA2/3 (07-598, Upstate/Millipore), GluN1 (a gift from Dr. R. Huganir), GluN2A (07-632, Upstate/Millipore), and GluN2B (71-8600, Invitrogen) antibodies. NMDAR subunit blots were developed using enhanced chemifluorescence substrate (ECF™ substrate, Amersham). AMPAR blots were probed simultaneously with GluR1 and GluR2/3 antibodies followed by second antibodies linked to Cy5 and Cy3. All blots were scanned using TYPHOON™ 9400 (GE Health), and quantified using Image Quant TL software (GE Health). The signal of each sample on a blot was normalized to the average signal from the control group to obtain the % of average control values, which were compared across groups using unpaired Student's t-test.

Statistical analyses. All data were analyzed with Graphpad PRISM® 4 software using either a 2-tailed t-test or ANOVA with Tukey's post-hoc test for multiple comparisons, with significance determined at p<0.05. Cumulative distribution plots were analyzed using the Kolmogorov-Smirnov test. Descriptive statistics were calculated with StatView 4.1 and expressed as mean±S.E.M. (Standard Error of the Mean).

Results

BTA-EG₄ decreases Aβ Levels in vitro and in vivo

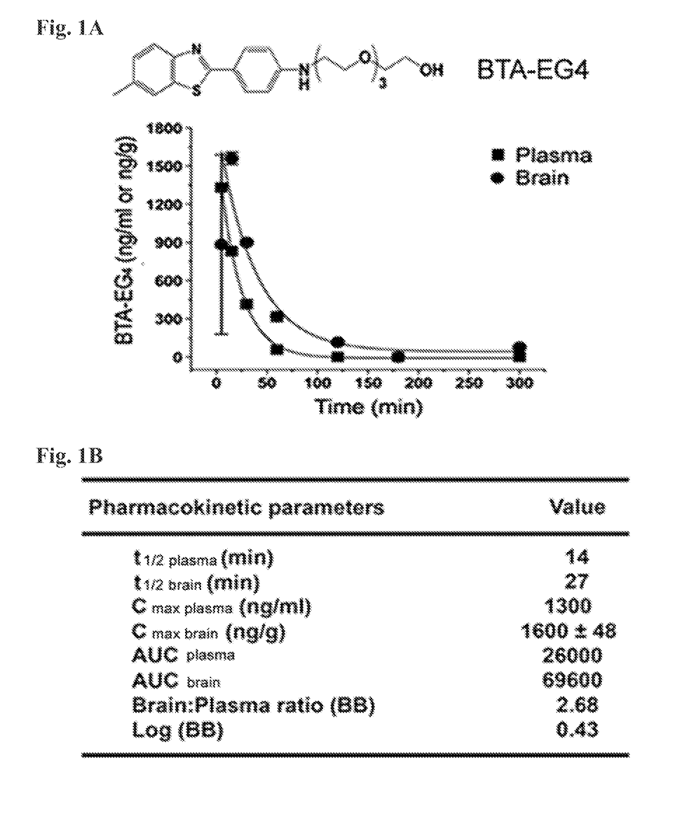

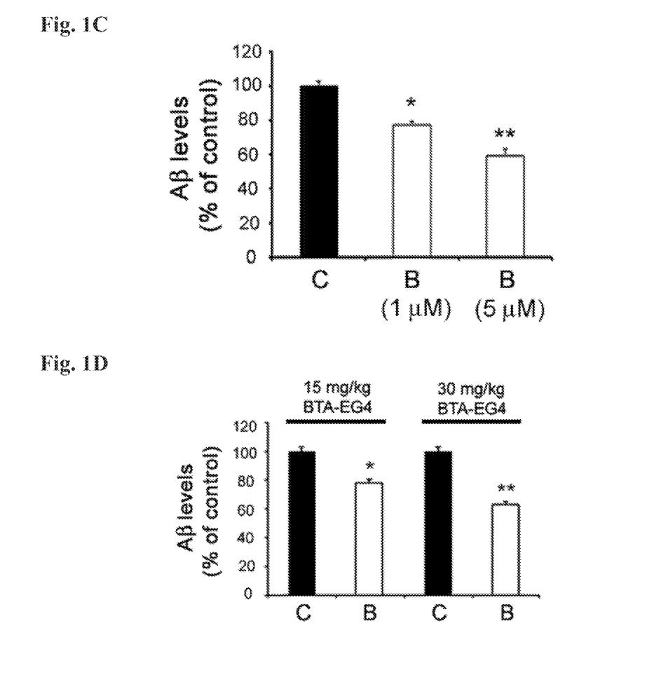

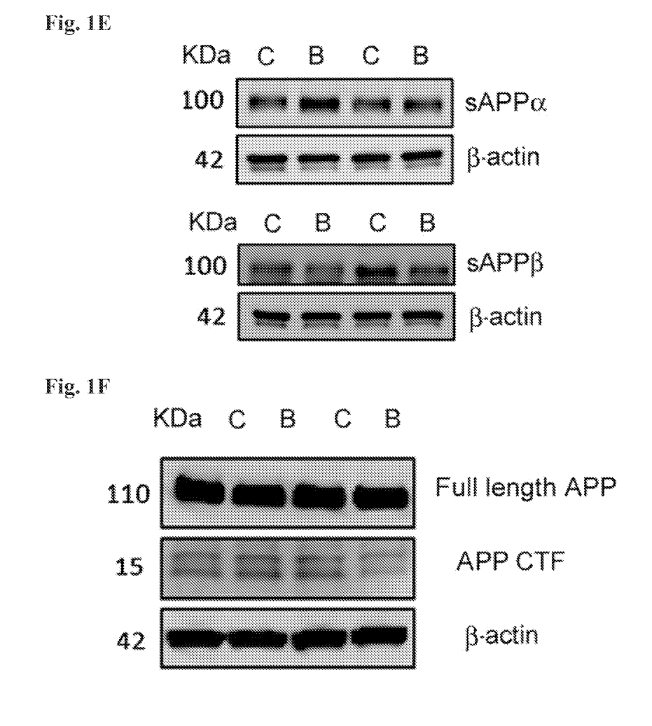

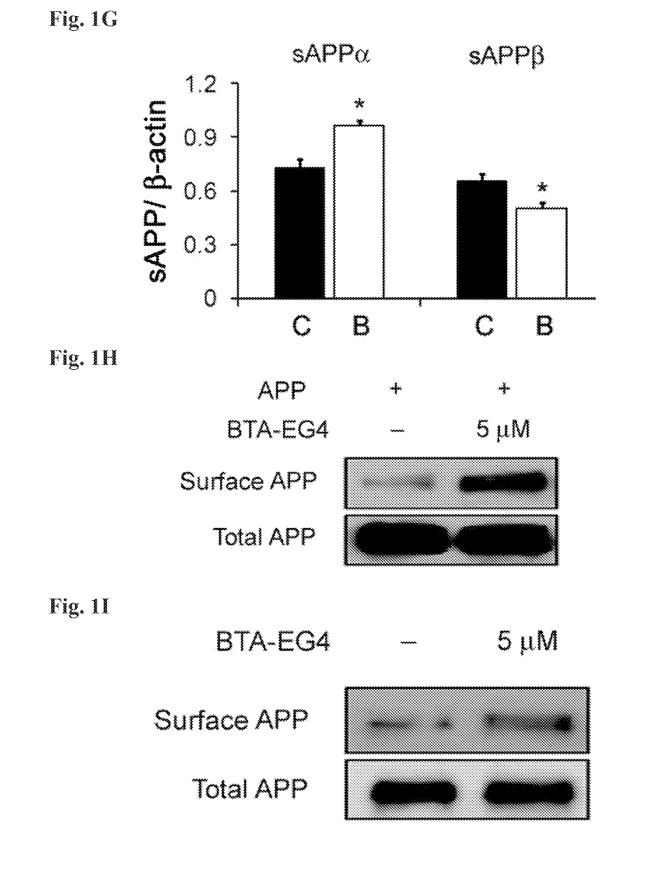

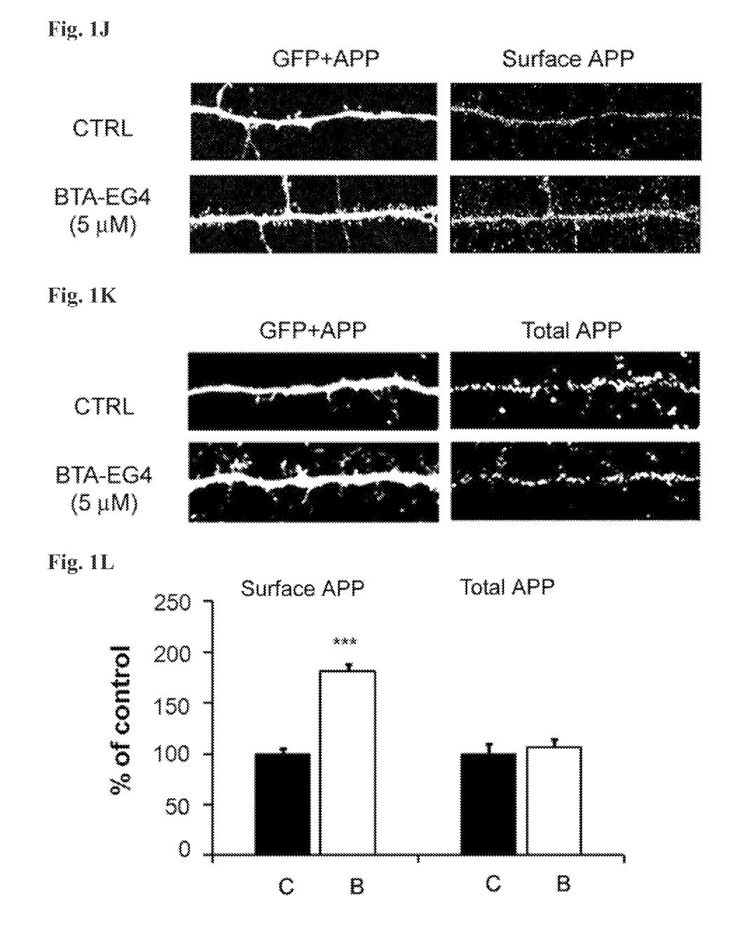

We examined the biological and pharmacokinetic properties of BTA-EG_{4.} BTA-EG₄ readily crosses the blood brain barrier and is rapidly distributed to the brain (Iyer et al., 2002) with an estimated logarithmic brain-to-plasma ratio (log BB) value of 0.43 (FIGS. 1A-1B). While it is clear from these studies that BTA-EG₄ readily distributes to the brain, we make this conclusion from the analysis of brain and plasma samples of only 4 mice at each time point (2 dosed with BTA-EG₄ and 2 dosed only with vehicle). The quantitative pharmacokinetic parameter values provided (FIG. 1B) should, therefore, be considered only as estimated values. We further found that daily injections of BTA-EG₄(≦50 mg/kg, i.p.) were well tolerated in wild-type mice for 16 days, and necropsy revealed no adverse effects on major organs.

To test whether BTA-EG₄ alters Aβ production in vitro, primary cortical neurons were treated with BTA-EG₄ (1 or 5 μM) or control (10% DMSO), and Aβ levels were measured using ELISA. BTA-EG₄ significantly decreased Aβ protein levels (FIG. 1C). We then examined whether BTA-EG₄ can alter Aβ levels in vivo by injecting wild-type mice daily for 2 weeks with BTA-EG₄ (15 or 30 mg/kg, 10% DMSO in saline, i.p.). We found that wild-type mice injected with both doses of BTA-EG₄ had significantly decreased Aβ peptide levels compared to controls (10% DMSO, i.p.) (FIG. 1D), suggesting that BTA-EG₄ can also reduce Aβ production in vivo. Furthermore, BTA-EG₄ altered APP processing in vivo, as monitored by increased sAPPα (α-secretase cleavage product) and decreased sAPPβ (β-secretase cleavage product) levels in BTA-EG₄ injected wild-type mice (30 mg/kg, i.p) compared to control-injected mice (FIGS. 1E, 1F). These findings suggest that BTA-EG₄ promotes α-secretase mediated metabolism of APP at the expense of β-secretase pathway, which may explain the reduction in Aβ.

It is well known that the majority of a-secretase activity occurs on the cell surface, while β- and γ-secretase activity occurs primarily in the early and late endosomes (Huse et al., 2000; Reiss et al., 2006). Thus, if APP is present at the cell's surface, it is preferentially cleaved by α-secretase, resulting in decreased Aβ production. Therefore, we examined whether BTA-EG₄ regulates cell surface expression of APP. To test this initially, COS7 cells were transfected with human APP and treated with BTA-EG₄ (5 μM) or control (10% DMSO) for 24 h. After performing cell surface biotinylation, we found that BTA-EG₄ increased cell surface APP (FIG. 1H). Furthermore, BTA-EG₄ increased endogenous cell surface APP levels in primary cortical neurons following 24 h of BTA-EG₄ (5 μM) treatment compared with control (10% DMSO) treatment (FIG. 1I). In an alternative approach to examine the effect of BTA-EG₄ on cell surface APP expression, we conducted live cell-surface immunostaining in primary hippocampal neurons. BTA-EG₄ treatment (5 μM) increased cell surface levels of APP relative to vehicle control without affecting total levels of APP (FIGS. 1J-1L). These results suggest that BTA-EG₄ may reduce Aβ production by increasing the amount of APP present at the cell surface.

BTA-EG₄ improves cognitive performance in the absence of enhanced LTP

Several studies have shown that Aβ accumulation contributes to cognitive deficits (Chang et al., 2011; Che'telat et al., 2012). Since we observed that BTA-EG₄ decreases Aβ levels both in vitro and in vivo (FIGS. 1C, 1D), we then examined whether BTA-EG₄ affects learning and memory.

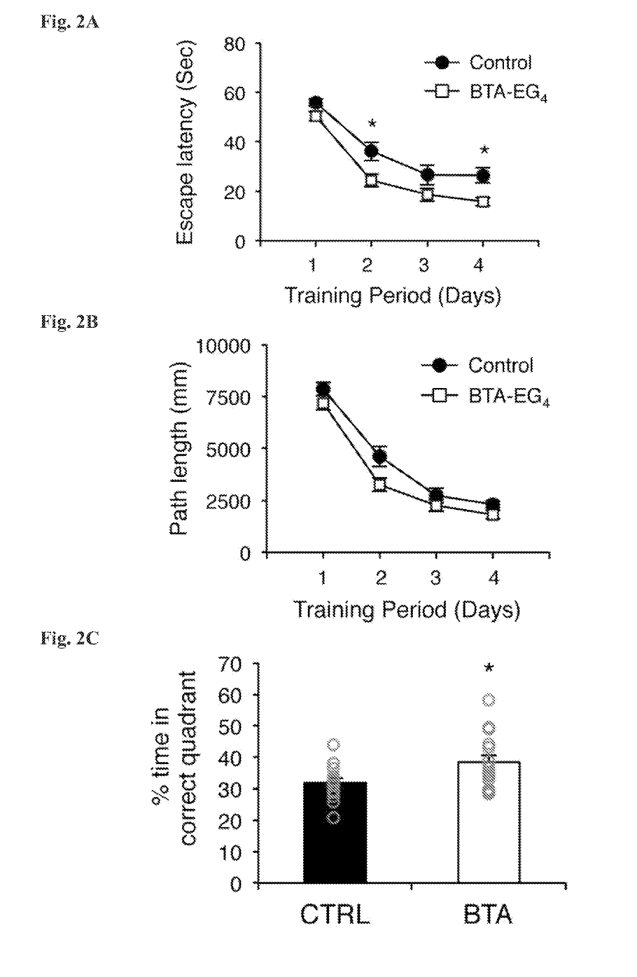

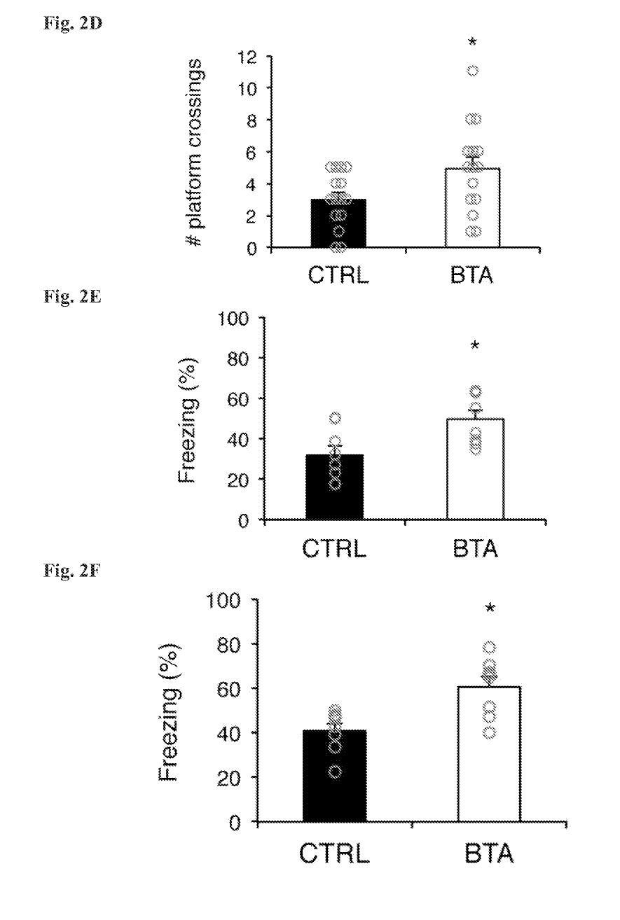

The Morris water maze task was used to assess cognitive performance in wildtype mice injected with BTA-EG₄ (30 mg/kg, i.p.) and controls. BTA-EG₄-injected wild-type mice exhibited significantly reduced escape latency during training (FIG. 2A), which was accompanied by an increase in swim speed (CTRL=117±3.1 mm/s, BTA=130±3.6 mm/s; p<0.01), but no difference in path length to the escape platform (FIG. 2B). These findings suggest that the apparent reduction in escape latency in the BTA-EG₄ group may simply be a reflection of the effect of the drug on swim speed. The fact that there is no change in the path length to reach the platform, which is a measurement not affected by swim speed, during the training trials suggests that BTA-EG₄ may not improve the learning process. To test whether BTA-EG₄ affects memory, we ran probe trials to measure the percentage of time spent in the correct quadrant and the number of platform crossings. We found that BTA-EG₄-injected wild-type mice spent more time in the target quadrant and showed a significantly higher rate of platform crossing during probe trials (FIGS. 2C, 2D), suggesting that BTA-EG₄ improves memory in this standard spatial memory task.

We also conducted a fear-conditioning paradigm as an alternative method to measure the effect of BTA-EG₄ on cognitive performance. We found that 30 mg/kg BTA-EG₄-injected wildtype mice showed significantly increased freezing in both the contextual and cued tests (FIGS. 2E, 2F), suggesting that BTA-EG₄ improves cognitive performance.

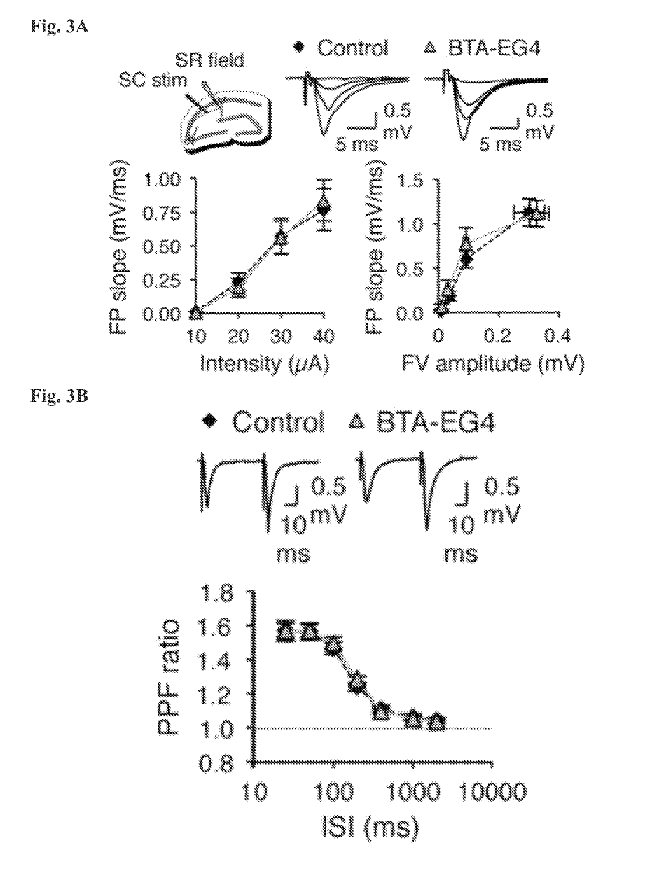

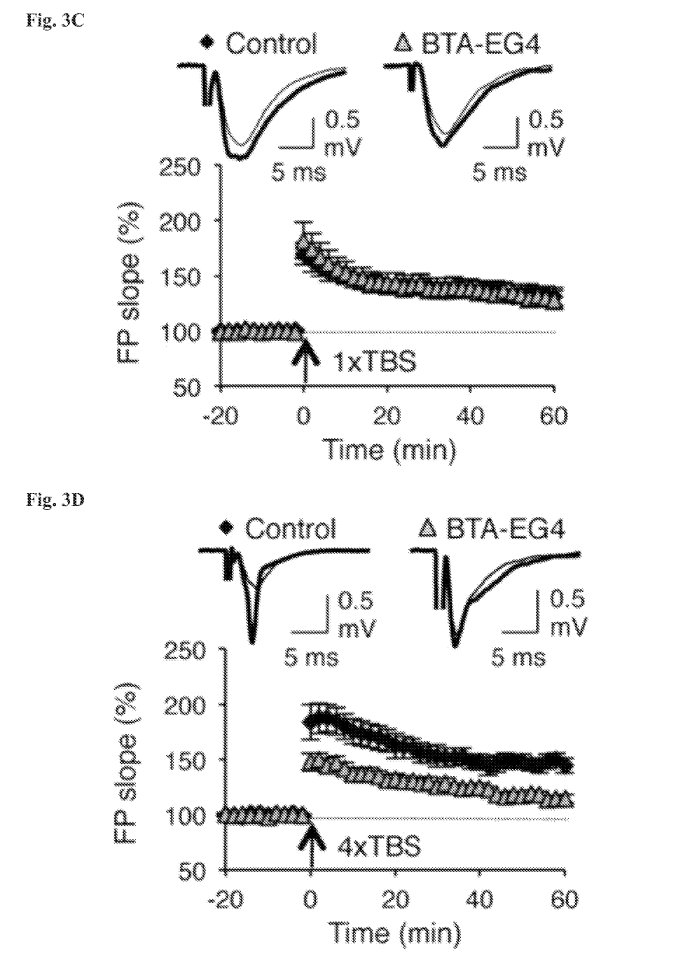

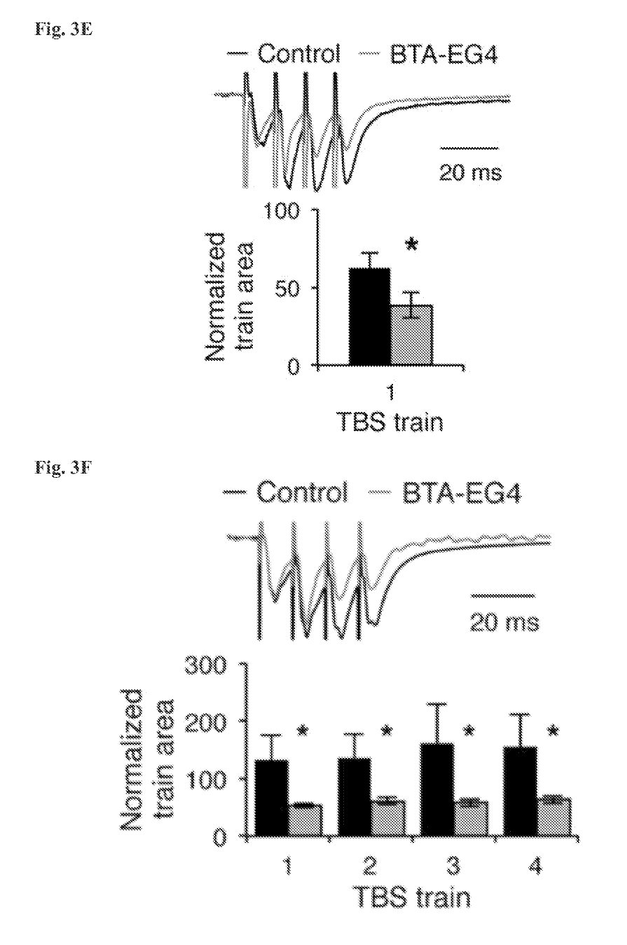

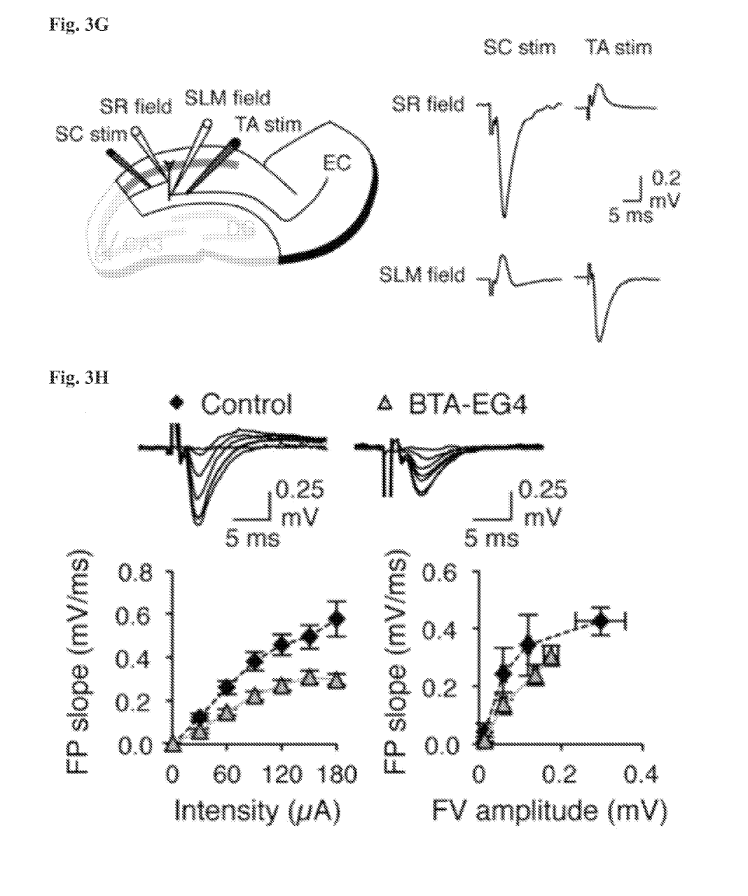

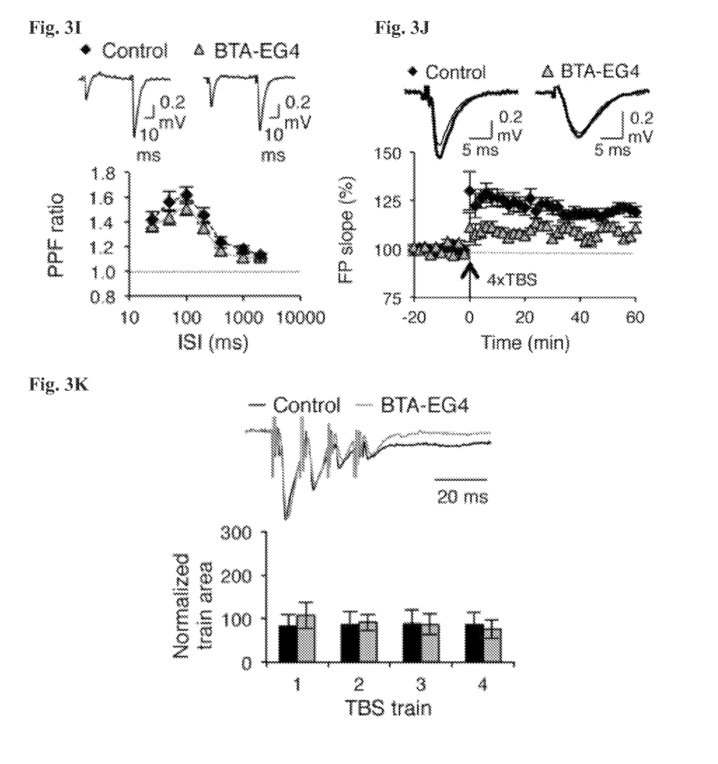

Several studies have demonstrated that synaptic plasticity is correlated with learning and memory (Kandel, 2001; VanGuilder et al., 2011). Therefore, we examined whether improved cognitive performance following BTA-EG₄ treatment is associated with altered synaptic function and plasticity. We conducted electrophysiology experiments in an acute hippocampal slice preparation from wild-type mice injected with BTA-EG₄ (30 mg/kg, i.p.) or control solution (10% DMSO, i.p) for 2 weeks. At the Schaffer collateral (SC) inputs to CAl, BTA-EG₄ did not alter basal synaptic transmission or presynaptic function (FIGS. 3A-3B). Long-term potentiation (LTP) was either normal or reduced, depending on the induction protocol (FIGS. 3C-3D). Unexpectedly, the summation of synaptic responses during the LTP induction protocol was reduced (FIGS. 3E-3F), which suggests that the normal LTP expression is likely due to an upregulation of a downstream signaling cascade. Recent evidence suggests that temporo-ammonic (TA) inputs to CA1, rather than SC inputs to CA1, support water maze-type learning (Nakashiba et al., 2008). BTA-EG₄ (30 mg/kg, i.p.) treatment reduced the ability to recruit presynaptic axons per stimulation intensity at TA inputs to CA1 (FIG. 3H, left), but there was no difference in synaptic transmission when responses were normalized to the presynaptic fiber volley amplitude (FIG. 3H, right). There was also not a change in presynaptic function (FIG. 3I). Similar to SC inputs to CA1, LTP at TA inputs also showed a similar reduction in the magnitude of LTP (FIG. 3J), which occurred in the absence of a change in the response summation during the induction protocol (FIG. 3K). Collectively, these results support the idea that the benefit of BTA-EG₄ on improved cognitive performance is not through enhancing LTP.

BTA-EG₄ increases spinogenesis in vivo

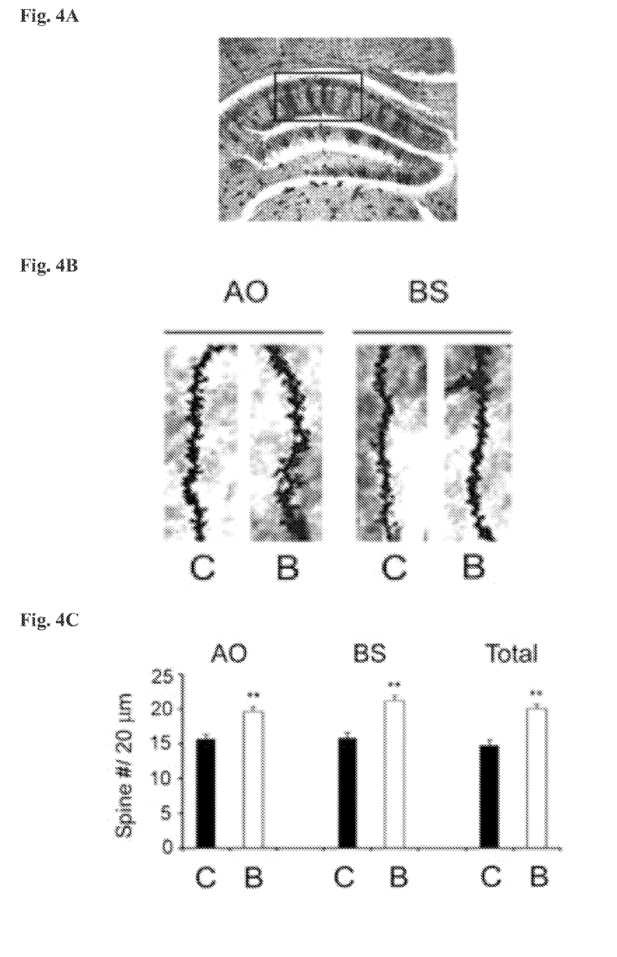

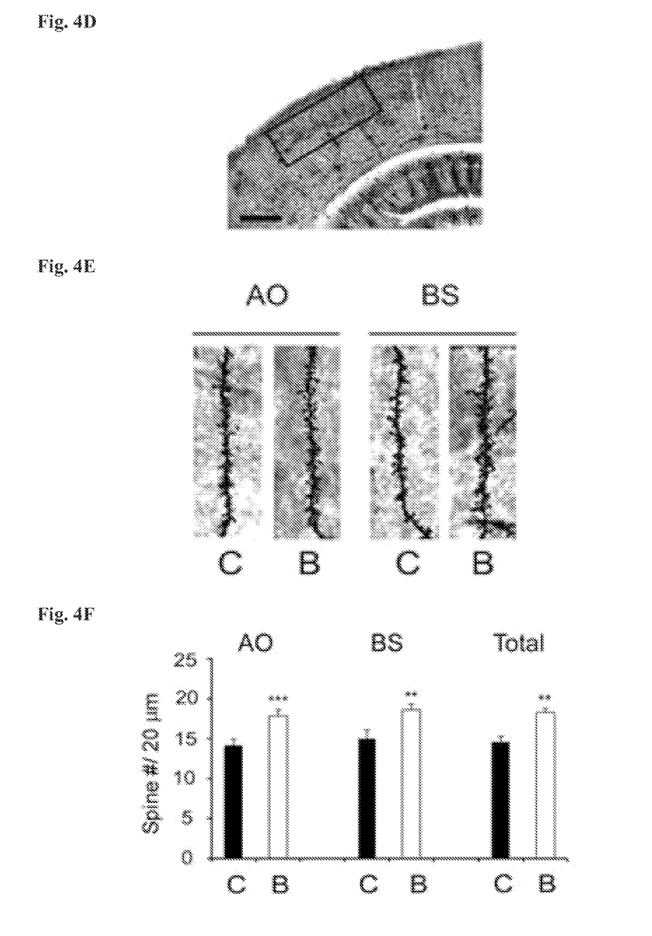

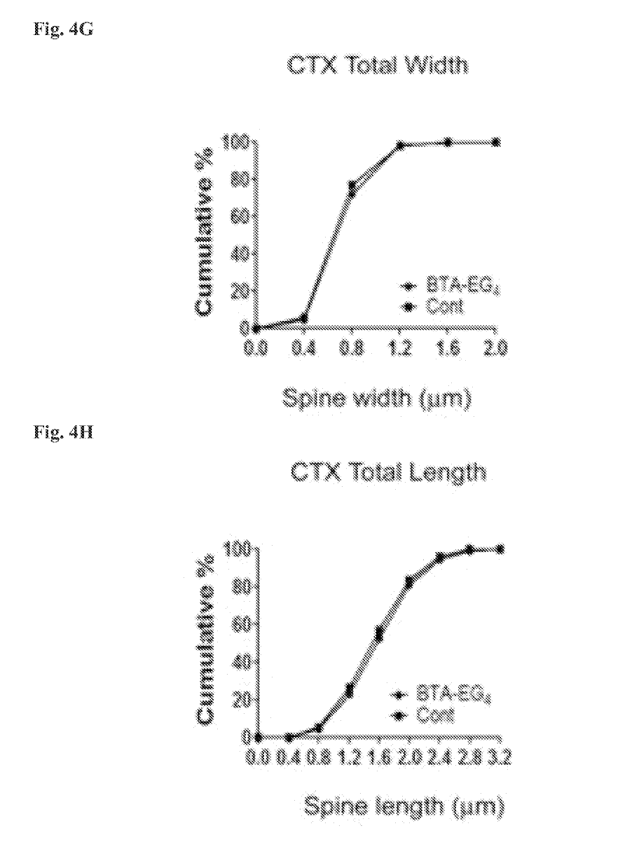

There is precedence that cognitive performance correlates better with dendritic spine density rather than LTP magnitude (Hayashi et al., 2004; Morgado-Bernal, 2011). Since we observed that BTA-EG₄ improves learning and memory without enhancing LTP, we hypothesized that BTA-EG₄ promotes cognitive performance by increasing spine density. To test this idea, wild-type mice were injected with BTA-EG₄ (30 mg/kg) or control for 2 weeks. Subsequently, we performed Golgi staining and found that BTA-EG₄ treated mice showed significantly increased dendritic spine density in the CA1 region of the hippocampus and cortical layers II/III (FIGS. 4A-4F). However, BTA-EG₄ did not alter spine morphology, including spine head width and spine length, in these areas (FIGS. 4G-4J). These data suggest that BTA-EG₄ promotes dendritic spine formation without affecting spine morphology.

BTA-EG₄ requires APP to increase dendritic spine density.

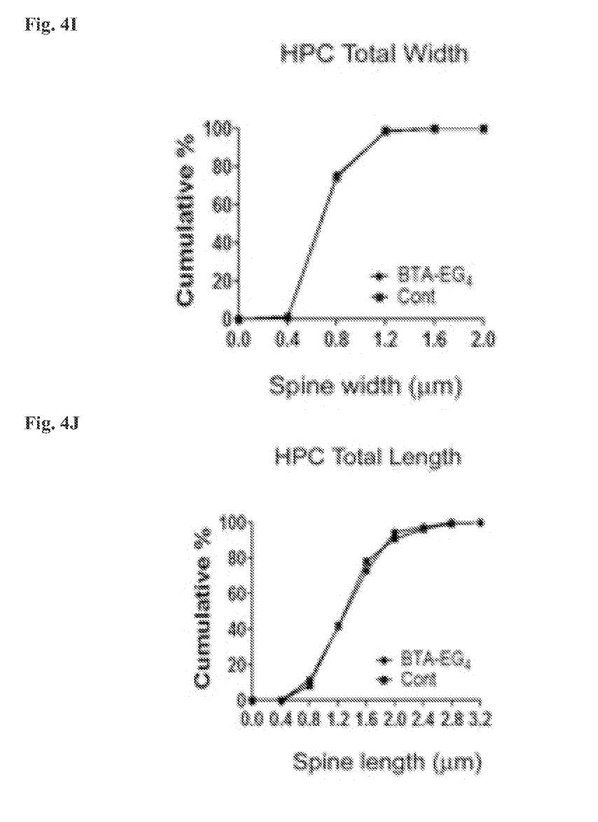

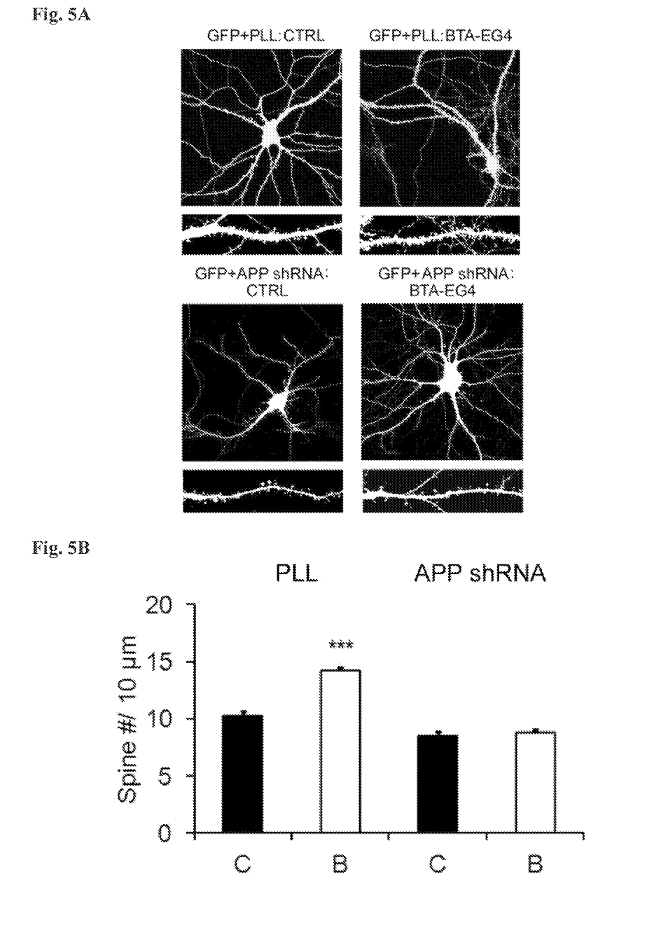

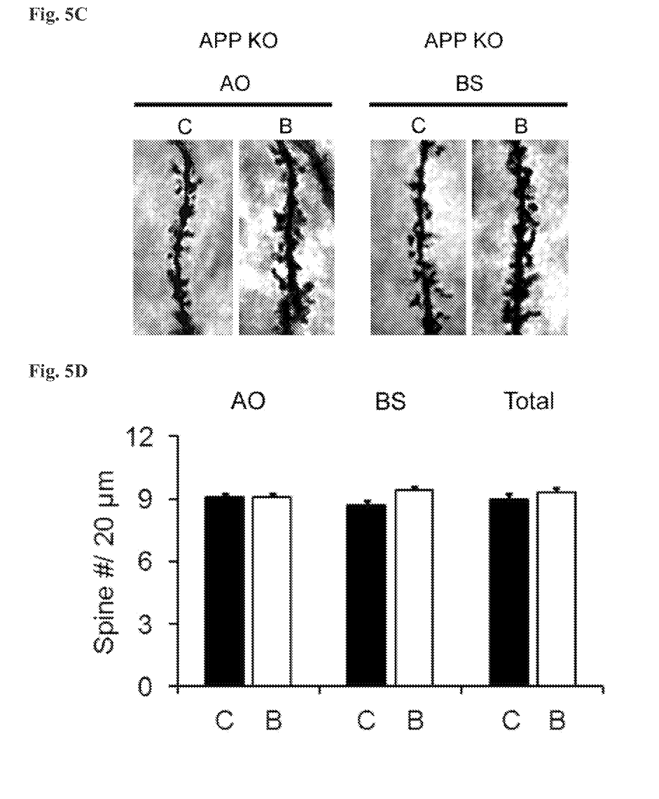

To examine whether BTA-EG₄ acts through APP to increase dendritic spine density, we acutely knocked down APP using shRNA in primary hippocampal neurons. APP shRNA was cotransfected with GFP to visualize dendritic spines, and control cultures were transfected with GFP and PLL (control vector for shRNA construct). We then treated both cultures with BTA-EG₄ (5 μM) or vehicle. We found that knockdown of APP decreased dendritic spine density on its own, and prevented the increase in dendritic spine density with BTA-EG₄ treatment (FIGS. 5A, 5B), which suggests that BTA-EG₄ can only increase dendritic spine density in the presence of APP. To confirm this finding in vivo, we examined the effect of BTA-EG₄ on dendritic spine density in APP knockout (KO) mice. APP-KO mice were injected with BTA-EG₄ (30 mg/kg) or vehicle for 2 weeks, and Golgi analysis was conducted on hippocampal CA1 neurons. In APP KOs, we did not find a statistically significant increase in dendritic spine density following BTA-EG₄ treatment (FIGS. 5C, 5D). This observation suggests that BTA-EG₄'s ability to promote spinogenesis is dependent on APP.

BTA-EG₄ increases AMPA mEPSC frequency but not amplitude

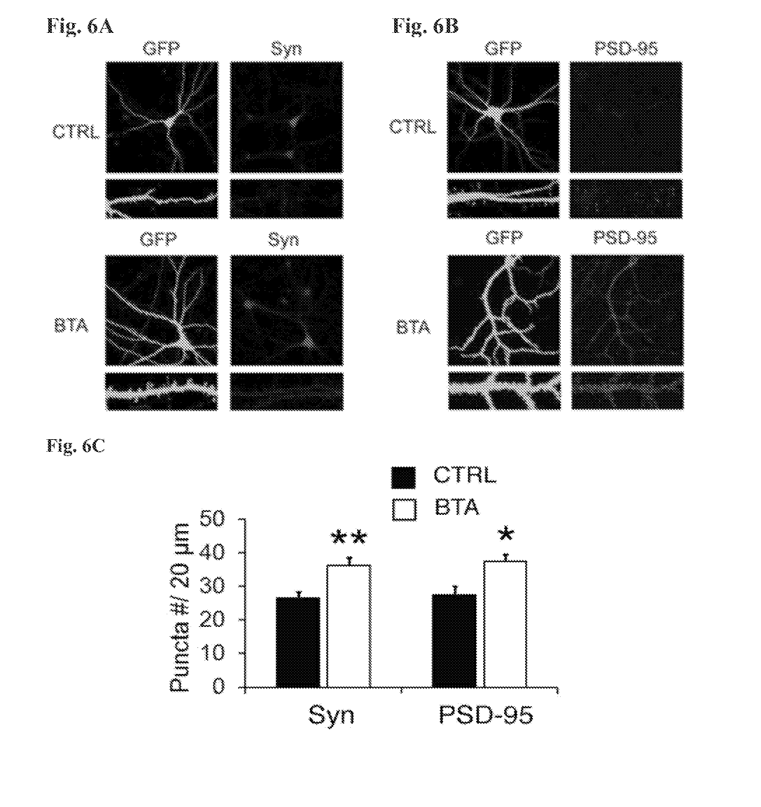

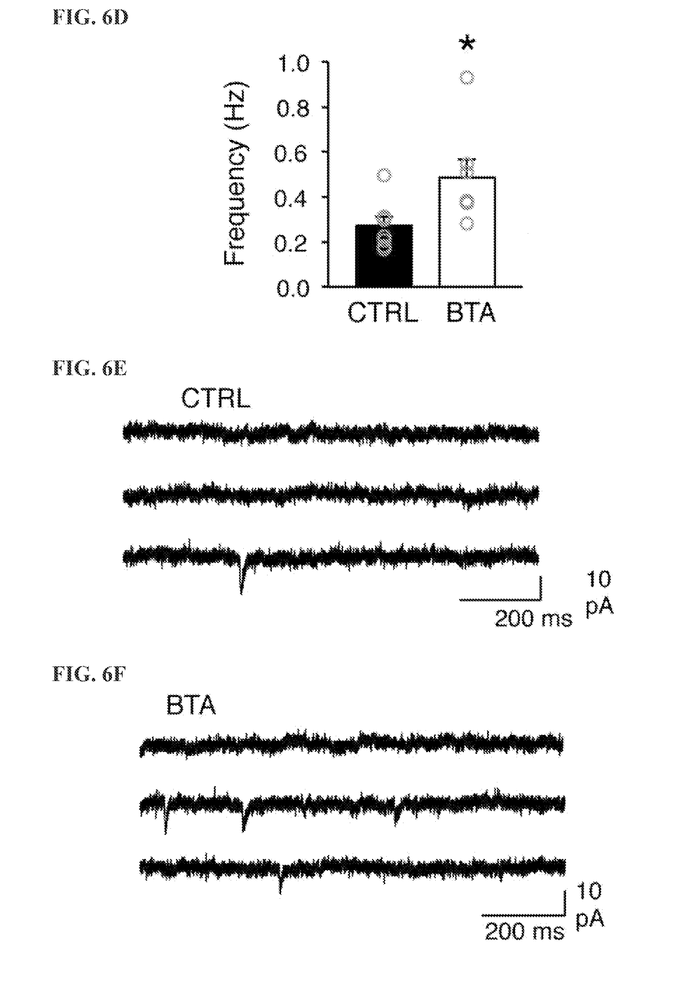

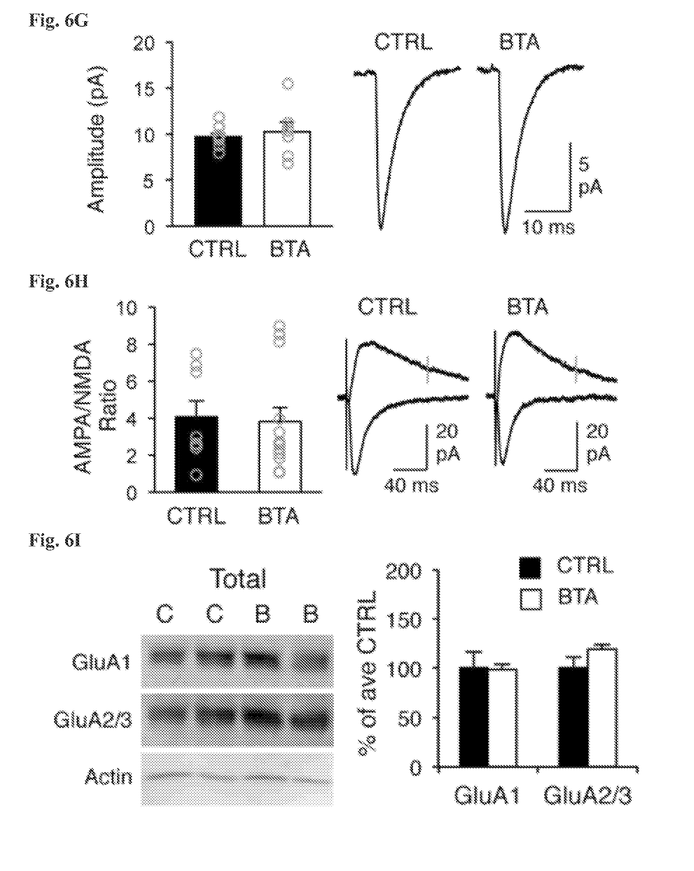

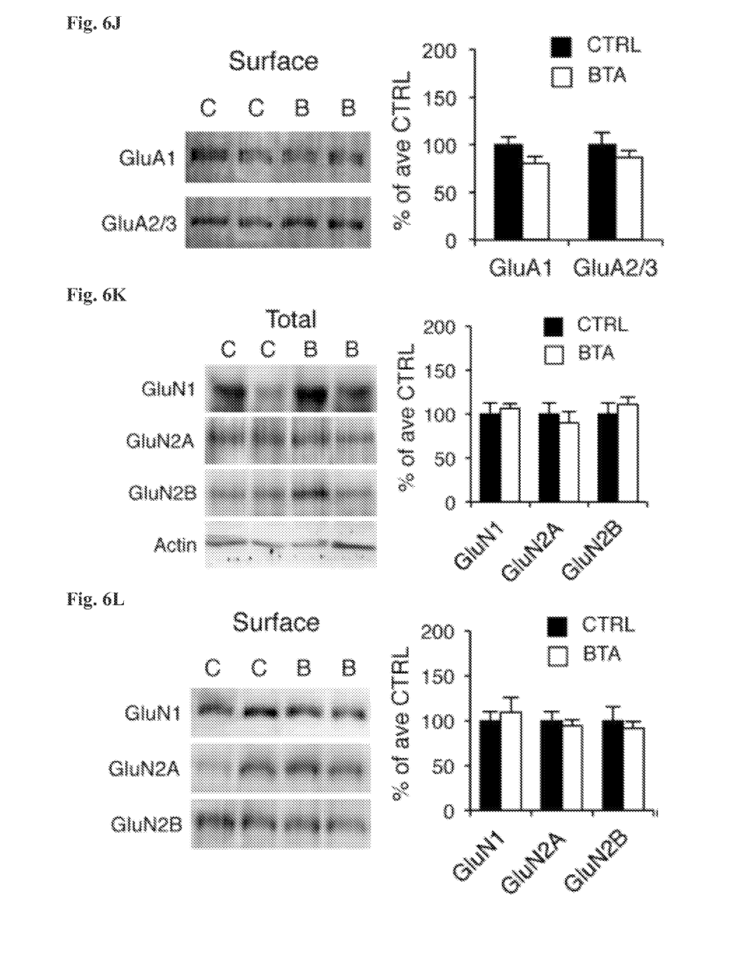

Next, we examined whether the increase in dendritic spine density following BTA-EG₄ treatment reflects an increase in the number of functional excitatory synapses. Primary hippocampal neurons were treated with BTA-EG₄ (5 μM) or control, and immunostained with synaptic markers. We found that BTA-EG₄ increased the number of puncta stained for synaptophysin (presynaptic marker) and PSD-95 (postsynaptic marker) (FIGS. 6A-6C).

To verify that the increase in dendritic spine density and synaptic proteins reflects an increase in functional synapses, we measured miniature excitatory postsynaptic currents (mEPSCs) from CA1 neurons in hippocampal slices following in vivo administration of BTA-EG₄ (30 mg/kg, i.p.) for 2 weeks. Consistent with an increase in functional synapses, BTA-EG₄ significantly increased the frequency of mEPSCs compared to vehicle treated controls (FIG. 6D). There was no significant difference in the average mEPSC amplitude (FIG. 6G) suggesting no postsynaptic alteration in synaptic strength. Furthermore, we did not observe a change in the AMPAR/NMDAR ratio (FIG. 6H), which suggests that the new synapses likely contain both NMDAR and AMPAR at normal levels.

To test whether the increase in functional excitatory synapses is via cell surface recruitment of glutamate receptors, we performed steady-state surface biotinylation using acute hippocampal slices obtained from mice injected with BTA-EG₄ (30 mg/kg) or control vehicle for 2 weeks. Both the cell surface and total levels of major AMPAR subunits GluA1 and GluA2, as well as NMDAR subunits GluN1, GluN2A and GluN2B, were quantified. There was no significant change in cell surface or total expression of any of these proteins in hippocampal slices from BTA-EG₄ treated mice (FIGS. 6I-6L). These data suggest that BTA-EG₄ does not regulate cell surface or total expression of AMPAR or NMDAR. Hence, the increase in the number of functional synapses is likely due to lateral recruitment of existing cell surface glutamate receptors to new spines.

BTA-EG₄ alters synapse formation through Ras signaling

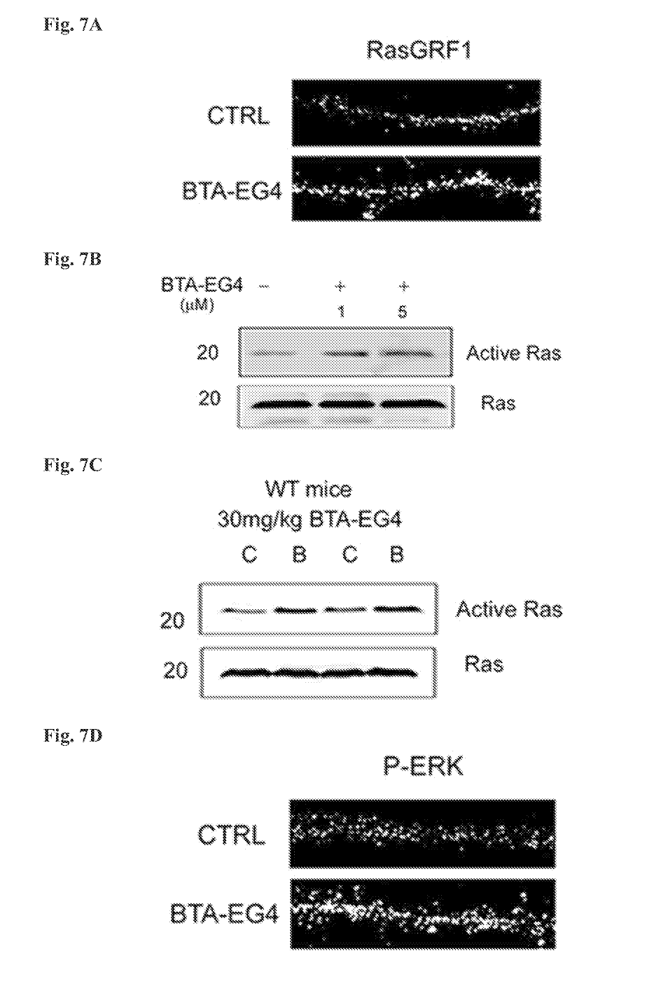

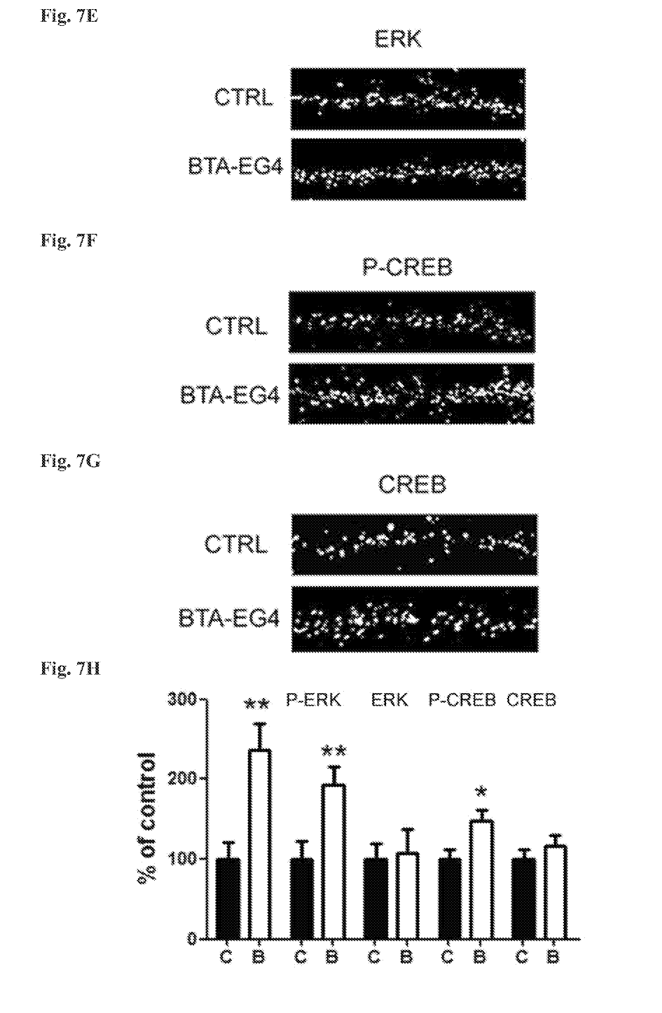

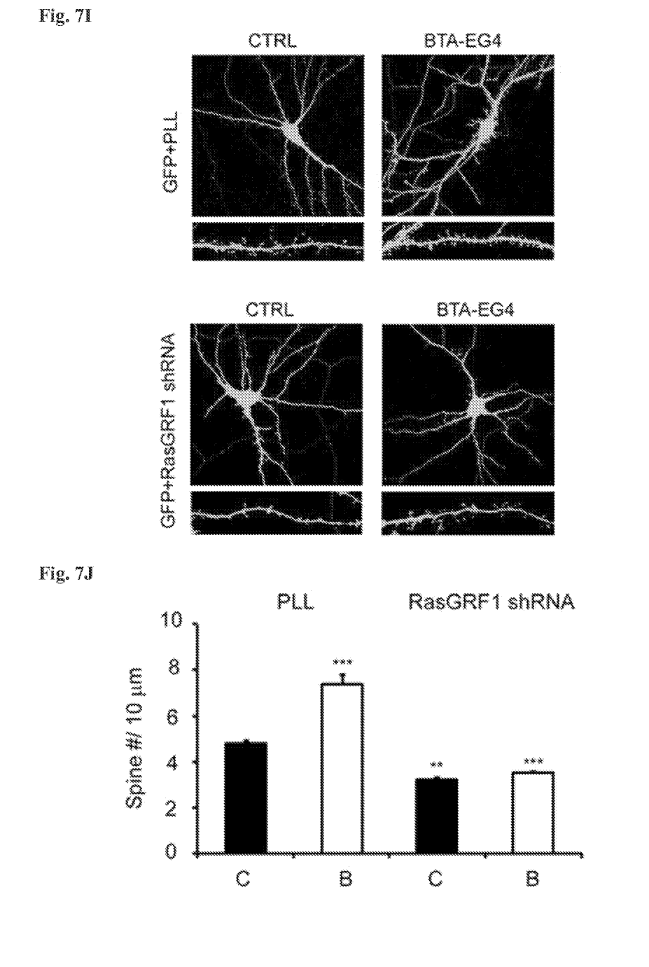

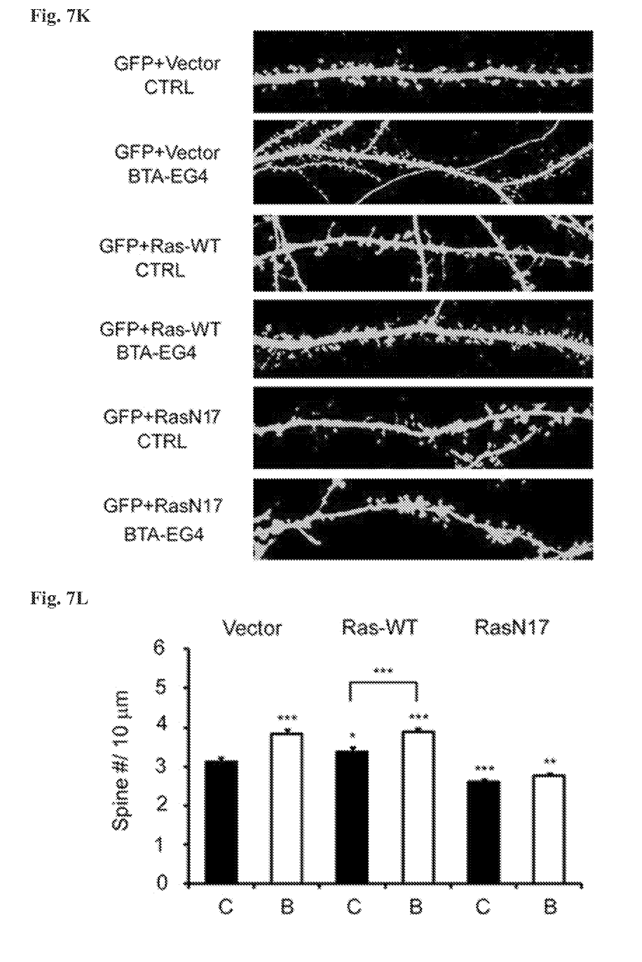

We next investigated the molecular mechanism by which BTA-EG₄ may alter dendritic spine formation. Ras, a small GTPase, is involved in dendritic spine formation and synaptic delivery of AMPA receptors (Zhu et al., 2002; Lee et al., 2011). Moreover, abnormal Ras signaling is associated with neurodegenerative disease, causing cognitive impairments and learning deficits (Stornetta and Zhu, 2011). Thus, we initially investigated the effect of BTA-EG₄ on Ras signaling by treating primary hippocampal neurons with BTA-EG₄ (5 μM) or control for 24 hrs. Interestingly, we found that BTA-EG₄ increased levels of RasGRF1, a guanine nucleotide exchange factor involved in Ras activation (Lee et al., 2010), as measured by immunofluorescence (FIGS. 7A-7B). Further, levels of active Ras were elevated following BTA-EG₄ treatment (5 μM) in primary cortical neurons (FIG. 7C) and following BTA-EG₄ treatment (30 mg/kg) in wild-type mice (FIG. 7D). We also examined whether BTA-EG₄ can alter the activity of downstream Ras signaling proteins, including p-ERK and p-CREB. We found that BTA-EG₄ (5 μM) increased the phosphorylation of ERK and CREB, the active forms of the signaling molecules downstream of Ras, without altering total ERK or CREB levels (FIG. 7E-L).

To examine whether the effect of BTA-EG₄ on dendritic spine formation is Ras dependent, primary hippocampal neurons were transfected with GFP and RasGRF1 shRNA, or GFP and PLL. After 24 h, we treated with BTA-EG₄ (5 μM) or control for another 24 h, and spine density was measured using immunofluorescence. Consistent with our findings above, BTA-EG₄ significantly increased dendritic spine density; however, RasGRF1 knockdown prevented the effect of BTA-EG₄ on dendritic spine formation (FIGS. 7I, 7J). In addition, primary hippocampal neurons were transfected with GFP and empty vector, GFP and Ras-WT, or GFP and RasN17 (inactive Ras mutant). After 24 h, we then treated neurons with BTA-EG₄ (5 μM) or control for 24 h, and spine density was measured. Ras-WT alone or combined with BTA-EG₄ increased dendritic spine density compared with control (FIGS. 7K, 7L). RasN17 decreased dendritic spine density compared with control, and BTA-EG₄ could no longer increase dendritic spine density in the presence of RasN17 (FIGS. 7K, 7L). These results suggest that Ras signaling is necessary for mediating the increase in dendritic spine density conferred by BTA-EG₄.

APP interacts with RasGRF1 and regulates Ras signaling proteins.

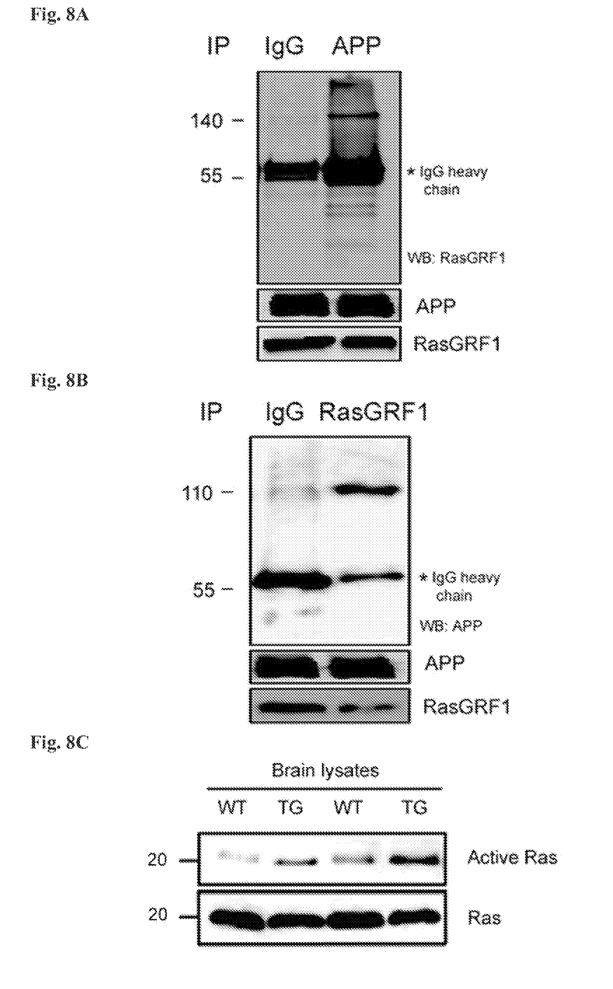

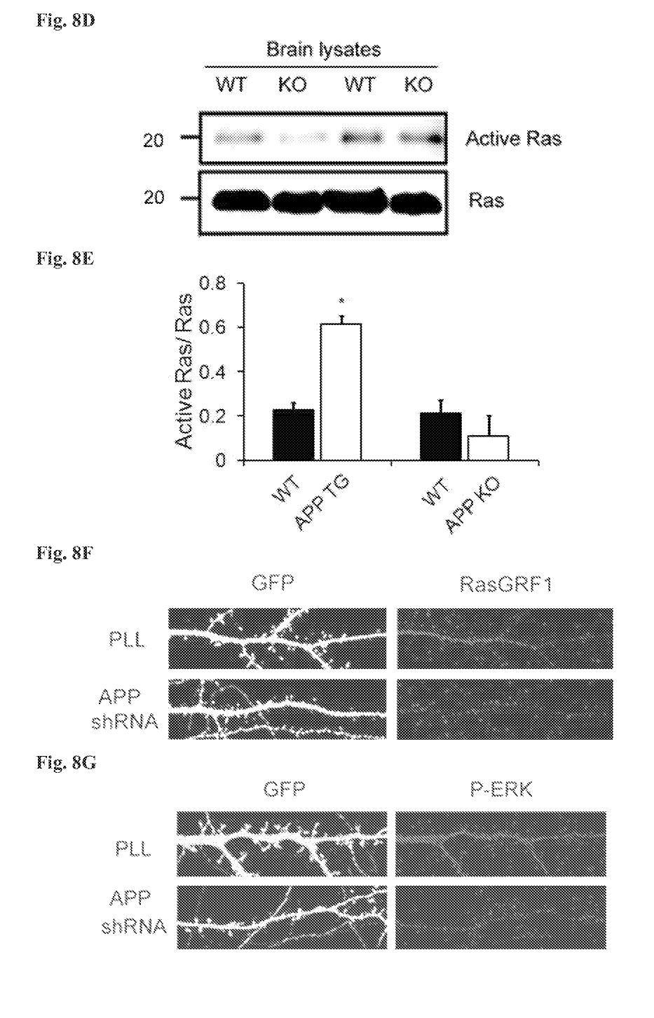

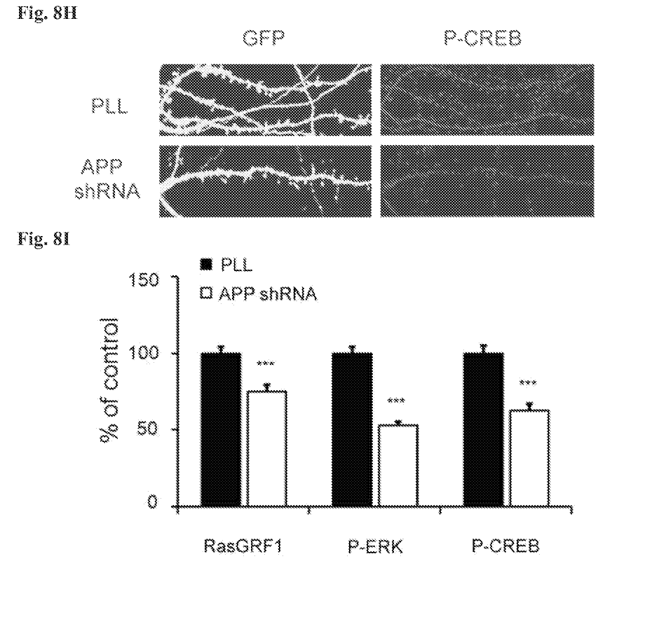

Since we observed that BTA-EG₄ functions through APP and requires Ras signaling to increase spine density, we examined whether APP can increase spine density through Ras signaling. To test this, we examined whether APP can interact with RasGRF1 by immunoprecipitating APP from brain lysates of wild-type mice, and probing for RasGRF1 (FIG. 8A). Interestingly, we found that APP co-immunoprecipitates with RasGRF1 (FIG. 8A). We also found that immunoprecipitating RasGRF1 pulls down APP (FIG. 8B). Our results indicate that RasGRF1 associates with APP in vivo.

Next, to examine whether APP can alter RasGRF1 levels, we examined the effect of APP on Ras activity in APP transgenic mice and APP KO mice using a GST pull-down assay (FIGS. 8C-8E). We found that Ras activity was elevated in 1-month-old APP transgenic mice (overexpressing APP without Aβ pathology at 1 month), but decreased in 10-month-old APP KO mice, compared with wild-type mice (FIGS. 8C-8E). Furthermore, we examined whether APP can alter the activity of downstream Ras signaling proteins ERK and CREB. First, to verify the effect of APP on Ras signaling, primary hippocampal neurons were transfected with GFP and PLL or GFP and APP shRNA, and then immunostained against RasGRF1. Knockdown of APP significantly decreased the levels of RasGRF1 compared with control vector (FIGS. 8F, 8I). We then immunostained primary hippocampal neurons transfected with GFP and PLL or GFP and APP shRNA against p-ERK and p-CREB. We found that knockdown of APP decreased the phosphorylation of ERK and CREB compared with control vector (FIGS. 8G-8I). These data suggest that APP may regulate dendritic spine formation through increases in Ras activity and downstream Ras signaling pathways.

BTA-EG₄ requires APP to alter Ras signaling.

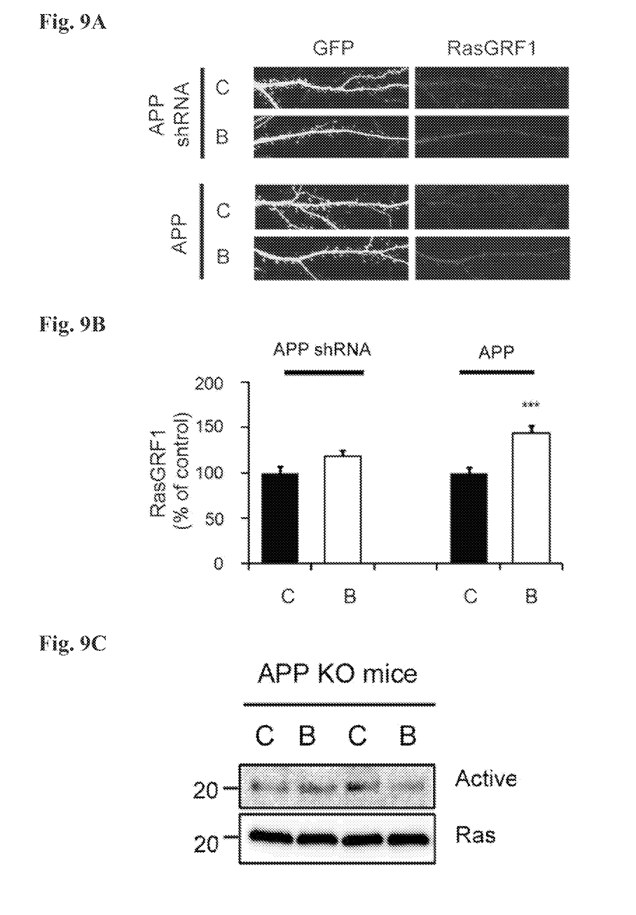

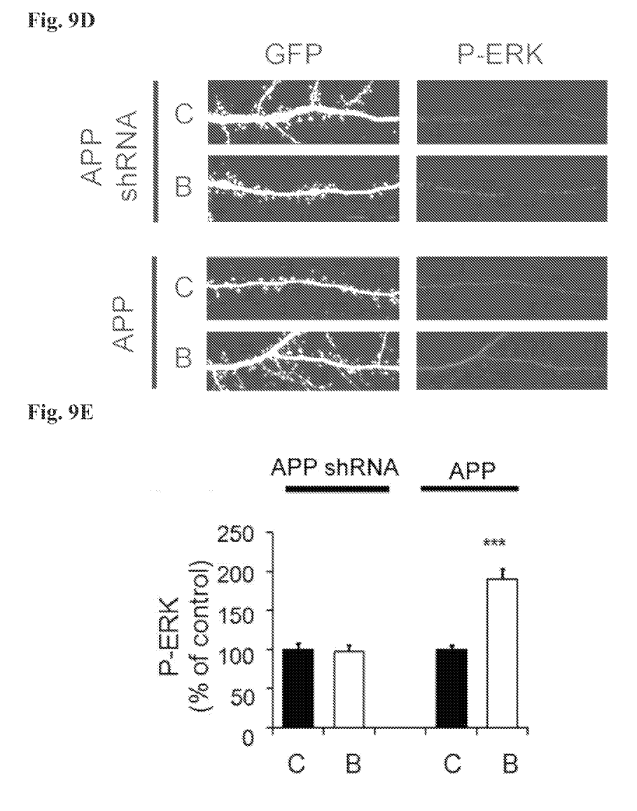

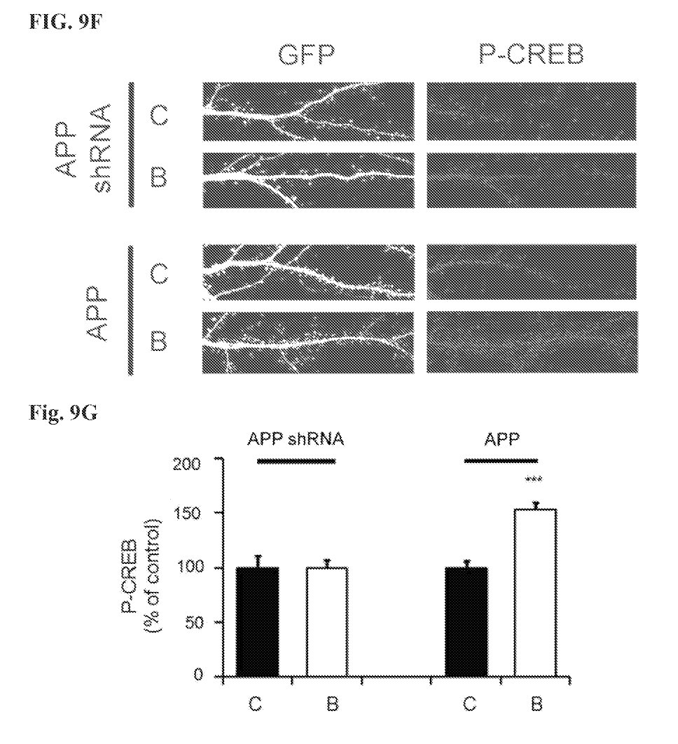

Since we observed that BTA-EG₄ and APP could possibly regulate dendritic spine density through Ras-dependent mechanisms, we then examined whether BTA-EG₄ requires APP to modulate Ras signaling. To test this hypothesis, primary hippocampal neurons were transfected with GFP and APP shRNA or GFP and APP, treated with control or BTA-EG₄ (5 μM), then immunostained against RasGRF1 (FIGS. 9A, 9B). We found that knockdown of APP did not increase RasGRF1 following BTA-EG₄ treatment compared with control, while overexpression of APP significantly increased RasGRF1 following BTA-EG₄ treatment compared with control (FIGS. 9A, 9B).

Next, we investigated whether BTA-EG₄ can alter Ras activity in APP KO mice by injecting control or BTA-EG₄ for 2 weeks. We found that BTA-EG₄-injected APP KO mice did not have altered Ras activity (FIG. 9C). Further, we examined the effect of BTA-EG₄ on downstream Ras signaling in the presence or absence of APP. For this experiment, primary hippocampal neurons were transfected with GFP and APP shRNA or APP, treated with BTA-EG₄ or control, and then immunostained against p-ERK or p-CREB. We found that BTA-EG₄ was ineffective at increasing p-ERK or p-CREB following knockdown of APP, while overexpression of APP significantly increased p-ERK and p-CREB with BTA-EG₄ treatment compared with control (FIGS. 9D-9G). These data strongly support that BTA-EG₄ acts via APP to activate Ras dependent signaling.

Discussion

In the present study, we demonstrate that the Aβ-targeting molecule BTA-EG₄ reduces Aβ levels by facilitating cell surface expression of APP. See e.g., FIG. 1A. Wild-type mice treated with BTA-EG₄ exhibited improved cognitive performance without enhancement of hippocampal LTP (FIGS. 3A-3K). Additionally, BTA-EG₄ promotes dendritic spine density, which was accompanied by an increase in the number of functional synapses as determined by elevated mEPSC frequency. Moreover, BTA-EG₄ regulates dendritic spine formation, potentially by increasing the activity of Ras-ERK signaling proteins through APP. See e.g., FIGS. 4A-9G). Without wishing to be bound by any theory, it is believed that, taken together, these results strongly suggest that BTA-EG₄ works through APP to increase dendritic spine density via a Ras ERK dependent mechanism. In addition, BTA-EG₄ warrants further investigation to determine its effect in mouse models of AD.

BTA-EG₄ treatment regulates APP metabolism, resulting in reduced Aβ levels and increases cell surface APP. It is known that β-secretase cleavage of APP forms Aβ along the intracellular endosomal pathway. Conversely, α-secretase cleavage of APP occurs at the cell surface and prevents Aβ production (Hyman, 2011). Because BTA-EG₄ did not alter the levels of Aβ degradation enzymes (i.e. insulin-degrading enzyme (IDE), neprilysin (NPE), data not shown), it is believed that BTA-EG₄ can decreases Aβ levels by specifically increasing cell surface levels of APP, and thus, favoring processing of APP by α-secretase cleavage over processing by β-secretase.

Several recent studies have shown that Aβ aggregation is correlated with deficits in learning and memory, and therapies that decrease Aβ can rescue these deficits (Loane et al., 2009; Chang et al., 2011). For instance, γ-secretase inhibitor (DAPT) injected mice had decreased Aβ levels and improved behavioral performance after traumatic brain injury (Loane et al., 2009). Other studies using mouse models of AD showed reduced Aβ levels after treatment with either β-secretase or HDAC inhibitors (Chang et al., 2011; Ricobaraza et al., 2011). These therapies were also able to prevent or improve memory deficits in AD mice. Furthermore, γ-secretase and HDAC inhibitors increase LTP, increase dendritic spine density, and improve cognitive performance (Townsend et al., 2010; Haettig et al., 2011). In contrast to these results, we found that while BTA-EG₄ had positive effects on cognitive performance and dendritic spine density, it did so without a correlated increase in the magnitude of LTP at both SC and TA inputs to CA1. This finding implies that BTA-EG₄ improves cognitive performance through an LTP-independent mechanism, and suggests that targeting spine density alone may be sufficient to improve cognitive function.

We found that BTA-EG₄ specifically acts to increase the number of functional synapses, but individual synapses are not stronger. The lack of an increase in LTP magnitude suggests that the new synapses are available for synaptic plasticity, but there is no enhancement of LTP due to the addition of new synapses. While our LTP findings defy the conventional interpretation of the role of LTP in memory formation, it is not an isolated case. Indeed, in the PAK transgenic model, decreased dendritic spine density was associated with a decrease in cognitive performance, but an enhancement of LTP magnitude (Hayashi et al., 2004). Combined with our results, this suggests that an increase in the number of dendritic spines and functional synapses confer benefits to cognitive function. The reduction in LTP magnitude seen in some cases may be a homeostatic adjustment to the increase in synapse number. For example, higher synaptic density may increase the LTP induction threshold to prevent over-excitation of neuronal activity. This presents an interesting point by implying that creating new synapses may benefit cognitive function not by enhancing LTP at individual synapses, but by allowing more synaptic contact points to form along the dendrite for potential information storage. It is of interest to note that some of the APP transgenic AD mouse models display larger LTP in younger age (Marchetti and Marie, 2011; Wang et al., 2012). It would be of interest to know whether BTA-EG₄ treatment in these young AD mouse models would show beneficial effects. While BTA-EG₄ significantly increased dendritic spine density in cortical layers II/III and the hippocampal CA1 region, this occurred without changes in dendritic spine morphology. Longer and thinner dendritic spines are characterized as immature “plastic” spines, while shorter and wider dendritic spines are characterized as mature memory spines (Kasai et al., 2002; Yasumatsu et al., 2008). Thus, BTA-EG₄ increases dendritic spine density without changing the proportion of immature and mature spines.

Here, we investigated the molecular mechanism by which BTA-EG₄ regulates dendritic spine density. One possibility is that BTA-EG₄ may act through a Ras-dependent mechanism because Ras signaling not only plays an important role in dendritic spine formation, but also in neuronal degeneration (Saini et al., 2009; Ye and Carew, 2010; Lee et al., 2011; Stornetta and Zhu, 2011). For instance, AD mice models have increased synaptic depression, which results in decreased activity and levels of RasGRF1, as well as downstream Ras signaling proteins. In contrast, AD patients have increased activity of Rap effectors, including p-JNK, which is accompanied by the removal of synaptic AMPA receptors (Savage et al., 2002; Zhu et al., 2002; Stornetta and Zhu, 2011). Interestingly, we observed that BTA-EG₄ promotes Ras-ERK signaling. BTA-EG₄ treatment increased RasGRF1 levels and Ras activity as well as activation of downstream Ras signaling proteins, including p-ERK. We found that Ras activity is necessary for spinogenesis induced BTA-EG₄, which suggests that one of the main signaling pathways involved in BTA-EG₄ action is via its ability to activate Ras. Therefore, BTA-EG₄ has the potential to reverse the decrease in Ras signaling seen in AD.

How does BTA-EG₄ activate Ras signaling to increase spine density? One possibility is that BTA-EG₄ binds directly to Aβ to prevent negative functional effects, resulting in protection against synapse loss. We also have data to demonstrate that BTA-EG₄ promotes cell surface expression of APP, which is known to increase dendritic spine formation (Lee et al., 2010). Here, our novel finding provides evidence that APP promotes spinogenesis through direct or indirect interaction with RasGRF1 to increase Ras activity and downstream signaling to promote spinogenesis. Furthermore, the action of BTA-EG₄ on dendritic spine formation and Ras activity both required APP. While this does not rule out the possibility that BTA-EG₄ acts via neutralizing Aβ, the more parsimonious explanation is that BTA-EG₄ promotes APP signaling to enhance Ras-dependent spinogenesis. Whether the effect of BTA-EG₄ on APP signaling is strictly through enhancing cell surface APP levels or preventing β-cleavage of APP, perhaps via direct binding to the Aβ domain of APP, remains to be investigated. Nevertheless there is evidence that Aβ and full-length APP often produce opposite signaling (Hoe et al., 2012); hence, the dual role of BTA-EG₄ in reducing Aβ and promoting APP signaling is likely conferring benefit to synaptic and cognitive function.

Without wishing to be bound by any theory, it is believed that, taken together, these results suggest that BTA-EG₄ treatment decreases Aβ levels and improves cognitive performance. Moreover, BTA-EG₄ increases dendritic spine density through APP and Ras-dependent mechanisms.

### REFERENCES

Capule C C &Yang J, 2012, Analytical Chemistry 84:1786-1791.

Chang W P, et al., 2011, FASEB J 25:775-784.

Chetelat G, et al., 2012, Neurodegener Dis. 10:141-144.

Finder V H & Glockshuber R, 2007, Neurodegener Dis 4:13-27.

Habib L. K., et al., 2010, J Biol Chem 285:38933-38943.

Haettig J, et al., 2011, Learn Mem 18:71-79.

Hayashi M L, et al., 2004, Neuron 42:773-787.

Hoe H S, et al., 2012, CNS Neurosci Ther 18(1):47-56.

Huse J T, et al., 2000, J Biol Chem 275:33729-33737.

Hyman B T, 2011, Arch Neurol 68:1062-1064.

Inbar P, et al., 2006, Chembiochem 7:1563-1566.

Iyer M, et al., 2002, Pharmaceut Res 19:1611-1621.

Kandel E R, 2001, Science 294:1030-1038.

Kasai H, et al., 2002, Nihon Shinkei Seishin Yakurigaku Zasshi 22:159-164.

Lee K J, et al., 2011, Small Gtpases 2:162-166.

Lee K J, et al., (2010, Neuroscience 169:344-356.

Loane D J, et al., 2009, Nat Med 15:377-379.

Lustbader J W et al., 2004, Science 304:448-452.

Marchetti C & Marie H, 2011, Reviews in the Neurosciences 22:373-402.

Minami S S, et al., 2010, Mol Neurodegener 5:16.

Morgado-Bernal I, 2011, Neuroscience 176:12-19.

Nakashiba T, et al., 2008, Science 319:1260-1264.

Nicoll J A, et al., 2003, Nat Med 9:448-452.

Orgogozo J M, et al., 2003, Neurology 61:46-54.

Pak D T, et al., 2001, Neuron 31:289-303.

Rall W., 1969, Biophys J 9:1483-1508.

Reiss K, et al., 2006, Pharmacol Ther 111:985-1006.

Ricobaraza A, et al., 2011, Front Biosci (Elite Ed) 3:1375-1384.

Saini D K, et al., 2009, Trends Pharmacol Sci 30:278-286.

Savage M J, et al., 2002, J Neurosci 22:3376-3385.

Selkoe D J, 2011, Nat Med 17:1060-1065.

Stornetta R L & Zhu J J, 2011, Neuroscientist 17:54-78.

Subbarao K V, et al., 1990, J Neurochem 55:342-345.

Townsend M, et al., 2010, J Pharmacol Exp Ther 333:110-119.

VanGuilder H D, et al., 2011, Neurobiol Dis 43:201-212.

Wang H, et al., (2012, Neural Plasticity 2012:272374.

Yasumatsu N, et al., 2008, J Neurosci 28:13592-13608.

Ye X. & Carew T J, 2010, Neuron 68:340-361.

Zhu J J, et al., 2002, Cell 110:443-455.

### Example 2

### A tetra(ethylene glycol) Derivative of Benzothiazole Aniline Ameliorates Dendritic Spine Density and Cognitive Function in a Mouse Model of Alzheimer's Disease

Abstract. We recently reported that the tetra(ethylene glycol) derivative of benzothiazole aniline, BTA-EG₄, acts as an amyloid-binding small molecule that promotes dendritic spine density and cognitive function in wild-type mice. This raised the possibility that BTA-EG₄ may benefit the functional decline seen in Alzheimer's disease (AD). In the present study, we directly tested whether BTA-EG₄ improves dendritic spine density and cognitive function in a well-established mouse model of AD carrying mutations in APP, PS1 and tau (APPswe; PS1M146V; tauP301L, 3× Tg AD mice). We found that daily injections of BTA-EG₄ for 2 weeks improved dendritic spine density and cognitive function of 3× Tg AD mice in an age-dependent manner. Specifically, BTA-EG₄ promoted both dendritic spine density and morphology alterations in cortical layers II/III and in the hippocampus at 6-10 months of age compared to vehicle-injected mice. However, at 13-16 months of age, only cortical spine density was improved without changes in spine morphology. The changes in dendritic spine density correlated with Ras activity, such that 6-10 month old BTA-EG₄ injected 3× Tg AD mice had increased Ras activity in the cortex and hippocampus, while 13-16 month old mice only trended toward an increase in Ras activity in the cortex. Finally, BTA-EG₄ injected 3× Tg AD mice at 6-10 months of age showed improved learning and memory; however, only minimal improvement was observed at 13-16 months of age. This behavioral improvement corresponds to a decrease in soluble Aβ 40 levels. Taken together, these findings suggest that BTA-EG₄ is beneficial in ameliorating the synaptic loss seen in early AD.

Introduction. Alzheimer's disease (AD) is a neurodegenerative disease associated with amyloid-β (Aβ) pathology in the brain that contributes to synaptic loss by interacting with cellular components in harmful ways (Finder and Glockshuber, 2007; Habib et al., 2010; Lustbader et al., 2004). Excitatory synapse number is directly correlated with the number of excitatory sites of neurotransmission known as dendritic spines. Dendritic spines act as sites of learning and memory in the brain. Transient thin spines are thought to represent molecular sites of learning, while the persistent wider spines may represent molecular sites of memory (Kasai et al., 2002; Yasumatsu et al., 2008). Additionally, age-dependent synapse loss is common to many transgenic mouse models of AD, including 3× Tg AD mice (Knobloch and Mansuy, 2008). Interestingly, several studies have indicated that dendritic spine loss may be correlated with cognitive impairment more strongly than Aβ plaque levels in AD (Knobloch and Mansuy, 2008; Masliah et al., 2006; Scheff and Price, 2006; Scheff et al., 2006; Selkoe, 2002; Terry et al., 1991). For instance, dendritic spine density is reduced in hippocampal region CA1 in patients with a diagnosis of early Alzheimer's disease (Scheff et al., 2007). Moreover, synapse number correlates with Mini Mental Status Exam (MMSE) score in layer III of the frontal cortex in human AD patients (Scheff and Price, 2006). Thus, measurement of dendritic spine morphology and density is used here to quantify synapse loss in the CA1 region of the hippocampus and cortical layers II/III in 3× Tg AD mice. Our previous research has demonstrated that members of the benzothiazole aniline (BTA) class of compounds directly interact with Aβ and can prevent Aβ-induced cytotoxicity (Capule and Yang, 2012; Habib et al., 2010; Inbar et al., 2006). Additionally, we found that a tetra-ethylene glycol derivative of BTA (BTA-EG₄) can penetrate the blood-brain barrier and has no toxicity (Capule and Yang, 2012; Inbar et al., 2006). Moreover, our recent work demonstrated that BTA-EG₄ alters normal synaptic function in vitro and in vivo by acting through amyloid precursor protein (APP) to target Ras-dependent spinogenesis (Megill et al., 2013). This increase in dendritic spine density and the number of functional synapses, as observed by elevated frequency of AMPA-receptor-mediated miniature excitatory postsynaptic currents (mEPSCs), was accompanied by improved memory in cognitive tasks (Megill et al., 2013).

In the present study, we examined whether BTA-EG₄ can improve synapse loss and cognitive deficits in a mouse model of AD. We report here that BTA-EG₄-injected 3× Tg AD mice demonstrate age-specific improvements in dendritic spine density and morphology in cortical layers II/III and the CA1 region of the hippocampus. We also found that Ras activity correlated with the age-dependent increase in dendritic spine density following BTA-EG₄ treatment. Moreover, at 6-10 months BTA-EG₄ substantially improved, while at 13-16 months BTA-EG₄ modestly improved, learning and memory after daily injection for 2 weeks. These results suggest that BTA-EG₄ warrants further investigation with a longer duration of treatment as a novel therapeutic option for AD patients to mitigate synaptic loss and cognitive impairment.

Materials and Methods

Synthesis of reagents. 2-(2-(2-(2-hydroxyethoxy)ethoxy)ethoxy)ethyl toluenesulfonate, 2-(2-(2-(2-iodoethoxy)ethoxy)ethoxy)ethanol, and BTA-EG₄ were synthesized as described herein for Example 1.

Animals. Male homozygous 3× Tg AD mice were generated from mutant PS-1 (M146V) knock-in mice by microinjection of the following transgenes under the control of the Thy 1.2 promoter: human APP (K670M/N671L) and tau (P301L) on a hybrid 129/C57BL6 background (Oddo et al., 2003). All animal maintenance protocols and experiments were approved by the Institutional Animal Care and Use Committee at Georgetown University.

Golgi staining and morphological analysis of dendritic spines. Dendritic spine density and morphology analyses in the brain were conducted using the FD RAPID GOLGISTAIN KIT™ (FD NeuroTechnologies, Ellicott City, Md., USA). Mouse brains were dissected, immersed in Solutions A and B (2 weeks, room temperature, dark conditions), and then transferred to Solution C (24 h, 4° C.). A VT1000S Vibratome (Leica, Bannockburn, Ill., USA) was then used to slice brains 150 μm thick. Bright-field microscopy acquired dendritic images by Axioplan 2 (Zeiss, Oberkochen, Germany). Scion image software (Scion Corporation, Frederick, Md., USA) allowed measurement of spine head width, spine length, and linear density of cortical layers II/III and CA1 of the hippocampus. Images were coded and analyzed blind to experimental conditions. To measure dendritic spine density, we included spines from 0.2 to 2 μmin length, with at least 25 apical oblique (AO) and basal (BS) dendritic observations averaged from each animal (n=4-5 brains for 2-3 months old/group and 6-10 months old/group, n=3 brains for 13-16 months old/group). To measure spine morphology, we included spines up to 1.4 μm in width and 3.2 μm in length.

Ras activity assay. Brain lysate from 3× Tg AD mice at 6-10 months or 13-16 months of age was homogenized with Ral buffer (25 mM Tris-HCl, pH 7.4, 250 mM NaCl, 0.5% NP40, 1.25 mM MgCl2, and 5% glycerol) to measure active Ras levels. Briefly, brain lysate was incubated with GST-Raf-RBD purified protein coupled with GLUTATHIONE SEPHAROSE™ (Amersham) overnight at 4° C. After 24 h, pellets were washed with Ral buffer and western blotting was conducted with anti-Ras.

Western blot and analysis. 3× Tg AD mice were injected with 30 mg/kg BTA-EG₄ or vehicle daily for 2 weeks. After 2 weeks, the cortex and hippocampus were dissected and homogenized with RIPA buffer. Brain lysates were reduced, boiled, and run on a polyacrylamide gel followed by transfer onto nitrocellulose membrane and incubation in the following primary antibodies: rabbit anti-RasGRF1 (Santa Cruz, C-20), rabbit anti-GluA1 (Millipore, ab1504),mouse anti-GluA2 (Neuromab), rabbit anti-p-ERK (Invitrogen, 36880), or p-Elk (Cell Signal, 9181). Primary antibodies were applied overnight, washed with TBS buffer (3 washes, 5 min each), and HRP conjugated secondary antibodies were applied for 1 h. After 1 h, membranes were washed with TBS buffer (3 washes, 5 min each) and proteins were visualized by affixing the blots to autoradiography film. The density of each band was then quantified using ImageJ software (National Institutes of Health, Washington, D.C.) as a percentage of control following normalization to β-actin.

Morris Water Maze. To examine the effects of BTA-EG₄ on learning and memory, we injected 2-3 month old, 6-10 month old, and 13-16 month old 3× Tg AD mice daily (i.p.) for 2 weeks before beginning behavioral testing as described previously (Minami et al., 2012). The control groupw as treated with vehicle solution (10% DMSO) while the experimental group was treated with BTA-EG₄ (30 mg/kg) daily for 2 weeks. Animals were kept on a fixed 12 hour light-dark cycle, and behavioral experiments were conducted during the light portion of this cycle. Specifically, experiments began at 9:30 AM and concluded before 4:00 PM. Briefly, the amount of time required for animals to locate a Plexiglas platform submerged in opaque water was measured in a large circular pool. The animal was randomly placed into one of four quadrants separated by 90°, and the platform was hidden in one of these quadrants (14 in. from the wall). TOPSCAN software tracked the time required (latency, limited to 60 s) to locate the hidden platform, and animals failing to locate the platform within 60 s were gently guided to the platform. On the first trial, animals were allowed to remain on the platform for 15 s. All subsequent trials limited platform time to 10 s, and a probe trial (60 s) was administered 24 h after the final learning trial. Time spent in the quadrant where the platform was previously located and number of platform crossings were recorded during this probe trial. As a control, 12 h after the last trial, animals were required to locate a clearly visible black platform placed in a new location.

Statistical analyses. All data were analyzed using either a 2-tailed t-test or 1-way ANOVA with Tukey's Multiple Comparison test for post-hoc analyses (Graphpad PRISM® software, GraphPad, La Jolla, Calif.). Significance was determined as p<0.05. To analyze the Morris Water Maze escape latencies during the training phase, we used 2-way ANOVA with Tukey's Multiple Comparison test for post-hoc analyses. Descriptive statistics were calculated with StatView 4.1 and expressed as mean±S.E.M.

Results.

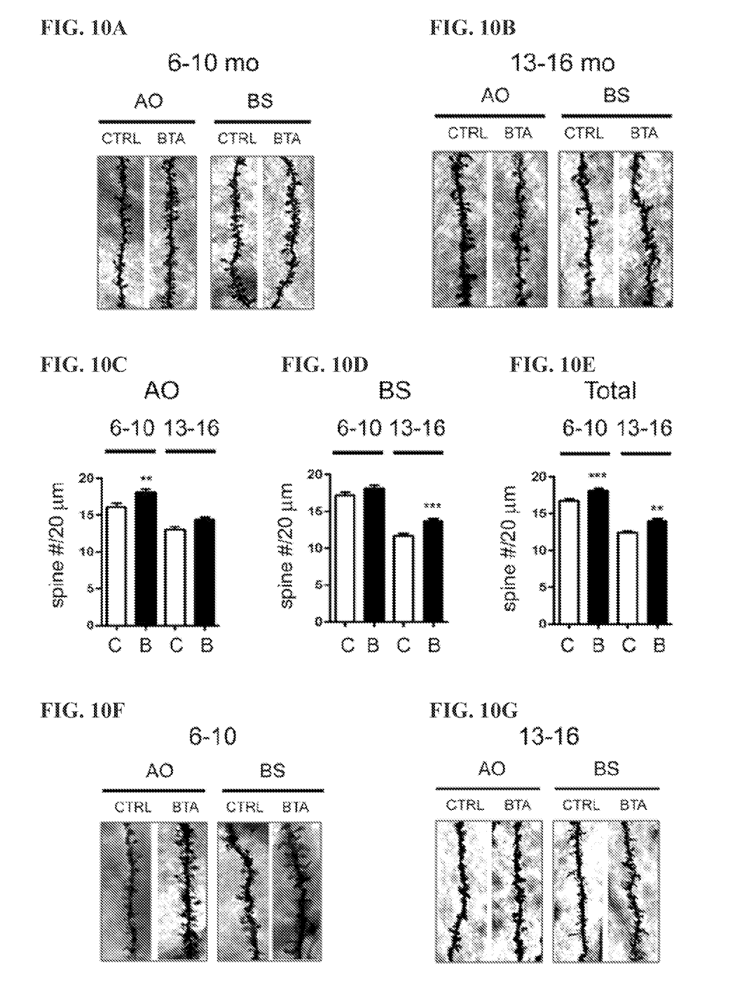

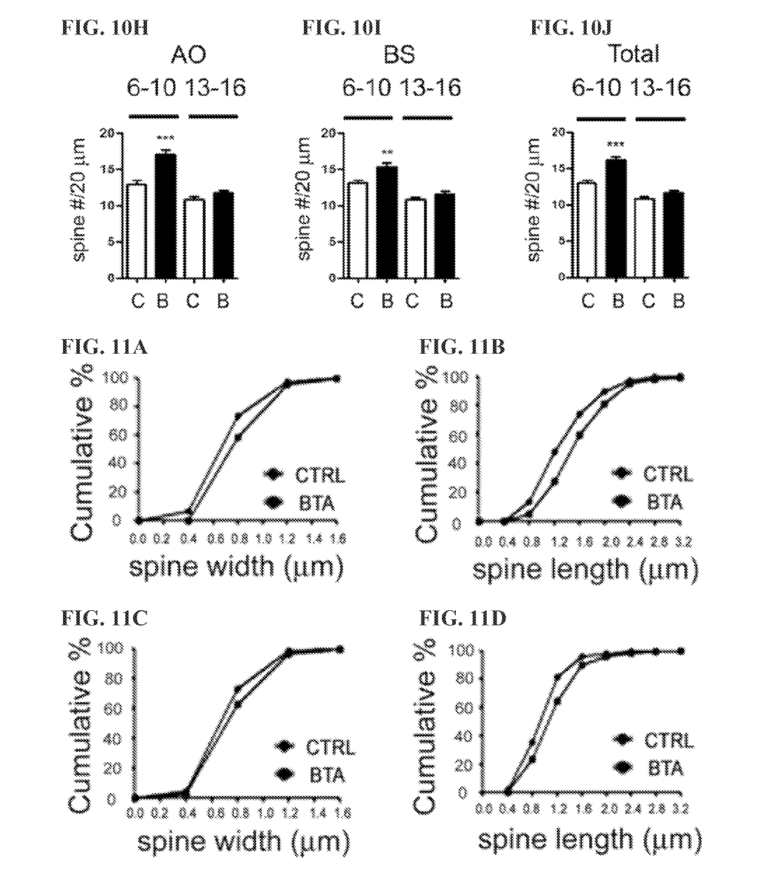

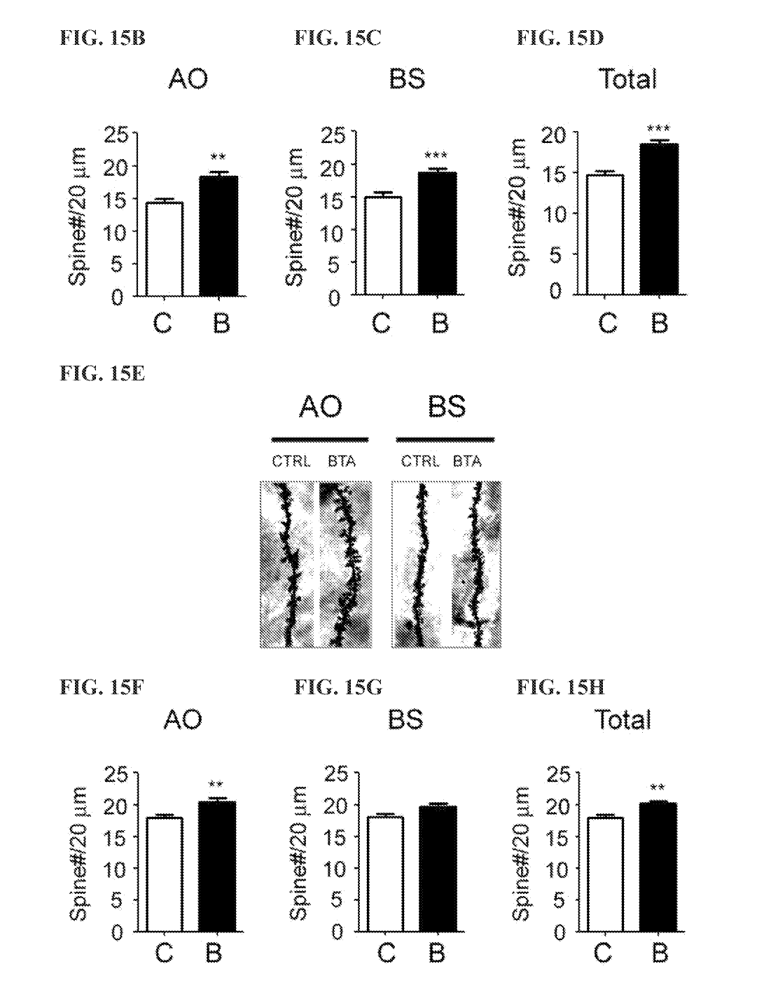

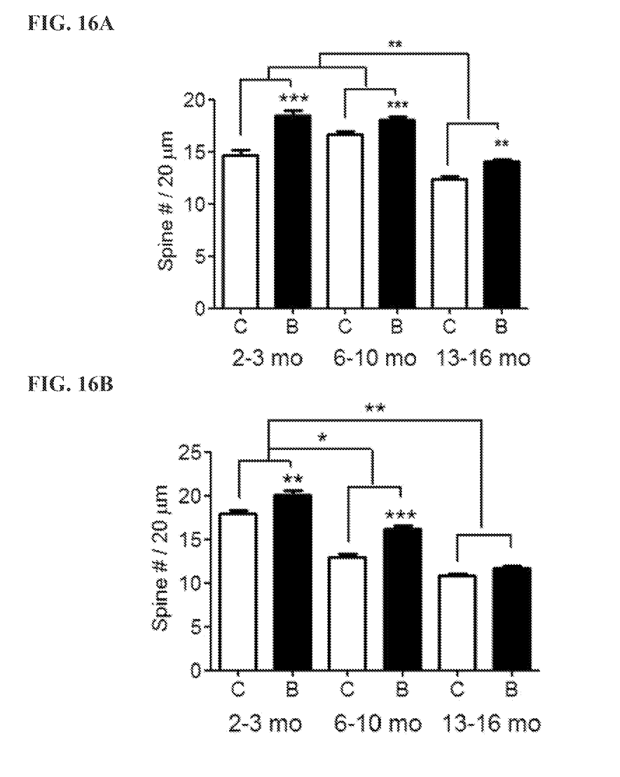

BTA-EG₄ alters dendritic spine density in the cortex and hippocampus. We recently reported that BTA-EG₄-injected wild-type mice exhibited increased dendritic spine density in the cortex and hippocampus (Megill et al., 2013). In the present study, we investigated whether BTA-EG₄ improves dendritic spine density in a mouse model of AD. For this study, we selected the 3× Tg AD mouse model due to its ability to model the progression of human AD (Oddo et al., 2003). This AD mouse model exhibits mild synapse loss at 6-10 months of age, and moderate loss by 13-16 months. To assess its effectiveness throughout disease progression, 2-3 month, 6-10 month, and 13-16 month old 3× Tg AD mice were injected with BTA-EG₄ (30 mg/kg, i.p.) or vehicle (10% DMSO) daily for 2 weeks. The 2-week duration of treatment was selected because it increased dendritic spine density and improved memory in wild-type mice (Megill et al., 2013). After the treatment, we conducted Golgi staining to measure dendritic spine density (FIGS. 10A-10J, FIGS. 15A-15H and FIGS. 16A-16B). We found that BTA-EG₄ injection significantly increased the overall density of dendritic spines in both the layers II/III of the cortex and the hippocampus CA1 at 2-3 months (n=4-5 brains/group) and 6-10 months of age (n=4-5 brains/group), but only improved the dendritic spine density of cortical neurons at 13-16 months of age (FIGS. 10A-10J, FIGS. 15A-15H and FIGS. 16A-16B, n=3 brains/group). There were subtle differences in the effect of BTA-EG₄ on dendritic spine density of apical oblique (AO) and basal (BS) dendrites at different ages, but overall our data suggest that the effectiveness of BTA-EG4 in improving dendritic spine density is reduced in older 3× Tg AD mice.

BTA-EG4-injected 3× Tg AD mice had wider and longer dendritic spines at 6-10 months old. Next, we further analyzed whether BTA-EG₄ could regulate dendritic spine morphology by measuring spine head width and spine length in cortical layers II/III and hippocampal region CA1. We found that 6-10 month old 3× Tg AD mice injected with BTA-EG₄ (30 mg/kg) had wider dendritic spines compared to vehicle-injected mice in the cortex and hippocampus using cumulative distribution analysis (FIGS. 11A, 11C) as well as comparison of average dendritic spine width (FIGS. 11I, 11K, n=4-5 brains/group). Additionally, 6-10 month old BTA-EG₄-injected 3× Tg AD mice also had longer dendritic spines in cortical layers II/III and hippocampus (FIGS. 11B, 11D, 11J, 11L, n=4-5 brains/group). However, neither the width nor length of dendritic spines changed when BTA-EG₄ was administered to 13-16 month old 3× Tg AD mice (FIGS. 11E-11H, 11I-11L, n=3 brains/group). Taken together, these data suggest that BTA-EG₄ treatment promotes dendritic spine density and alters dendritic spine morphology in cortical layers II/III and the CA1 region of the hippocampus of 3× Tg AD mice at 6-10 months of age, which is before severe Aβ plaque deposition and synapse loss.

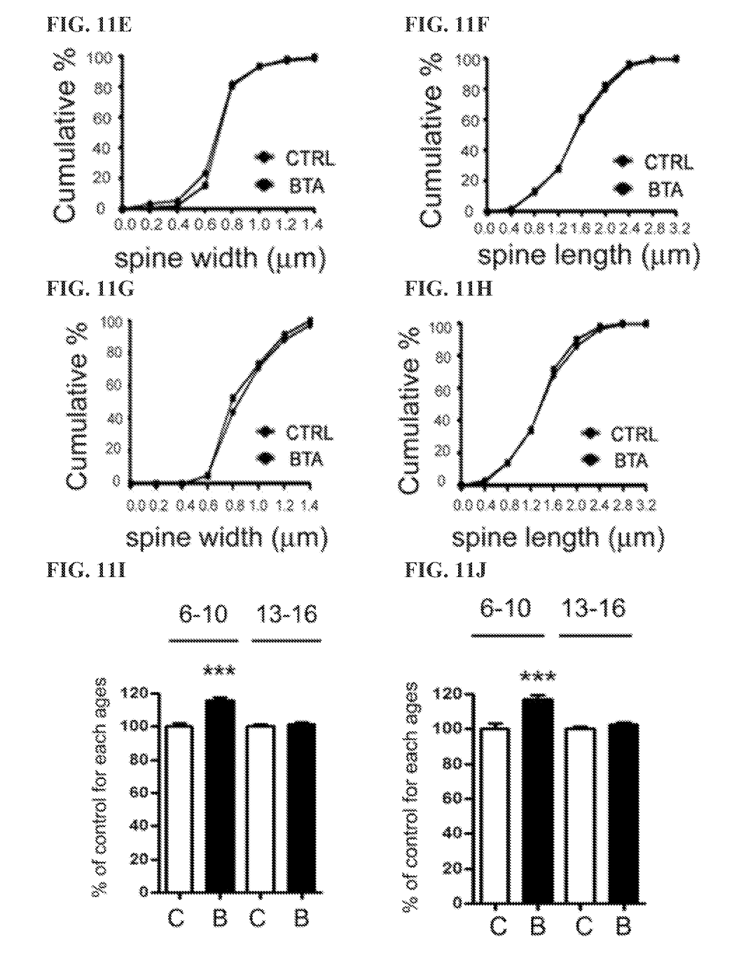

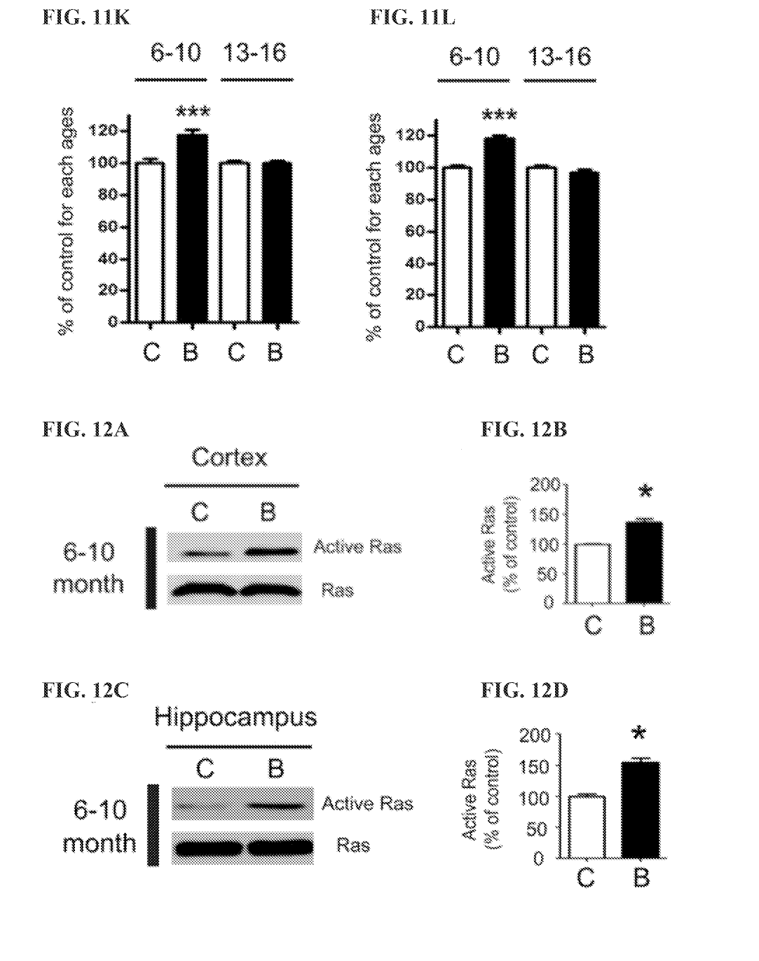

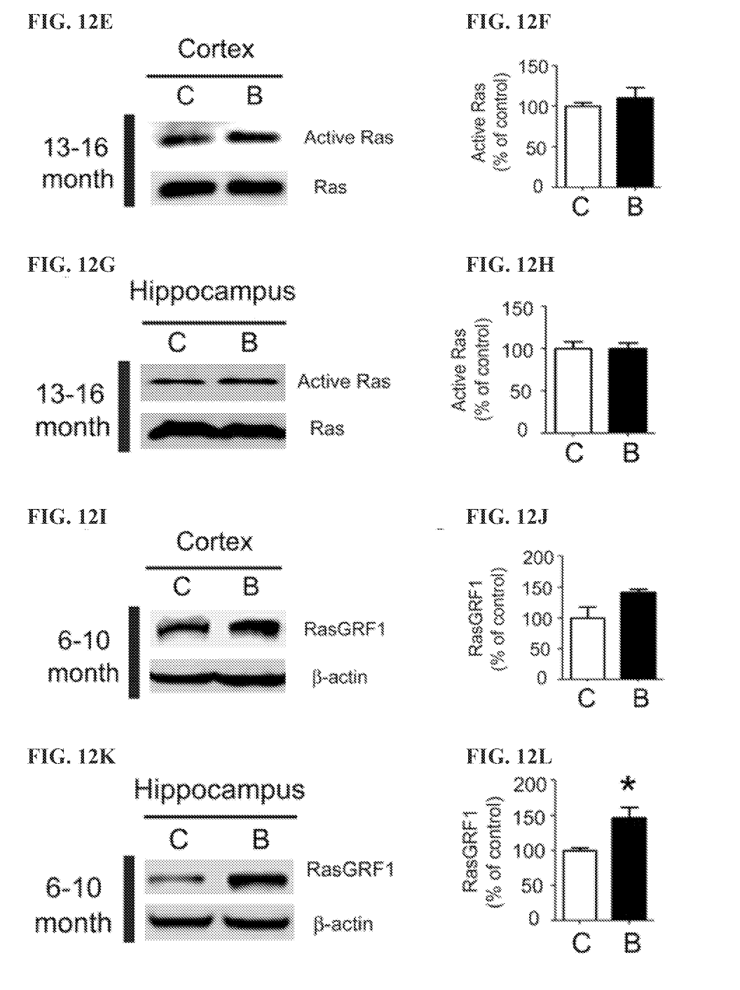

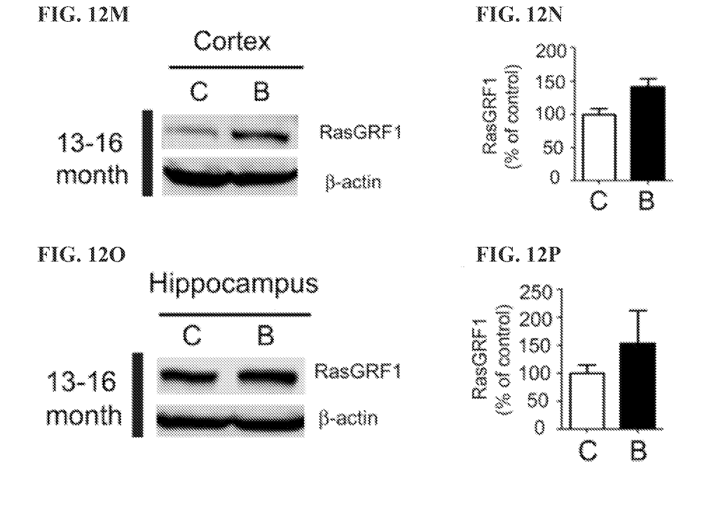

BTA-EG₄ injected 6-10 month old 3× Tg AD mice had increased Ras activity. Our recent study demonstrated that BTA-EG₄ promoted dendritic spine density in a Ras-dependent manner in wild-type mice (Megill et al., 2013). Therefore, we examined whether the same mechanism is shared for improving dendritic spine density in the 3× Tg AD mouse model. To test this, 6-10 month old or 13-16 month old 3× Tg AD mice were injected with 30 mg/kg BTA-EG₄ or vehicle daily for two weeks. After two weeks, mouse brains were homogenized with Ral buffer and levels of active Ras and RasGRF1 (a Ras effector) were measured via Western blot. We found that Ras activity was increased in the cortex and hippocampus of 6-10 month old BTA-EG₄-injected 3× Tg AD mice (FIGS. 12A-12D, n=2 brains/group). However, Ras activity was unchanged in the cortex and hippocampus of 13-16 month old mice (FIGS. 12E-12H, n=2 brains/group). Ras activity changes with BTA-EG₄ were corroborated by similar age specific increases in the level of RasGRF1 (FIGS. 12I-12P, n=3-4 brains/group). These data suggest that BTA-EG₄ may promote dendritic spine density in 3× Tg AD mice by modulating the Ras activity.

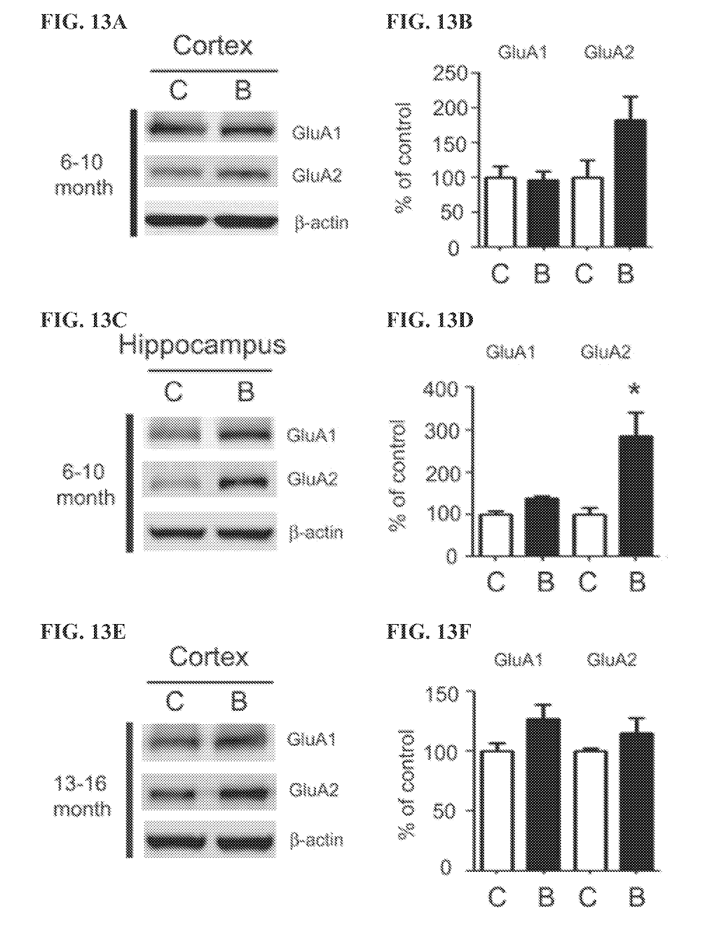

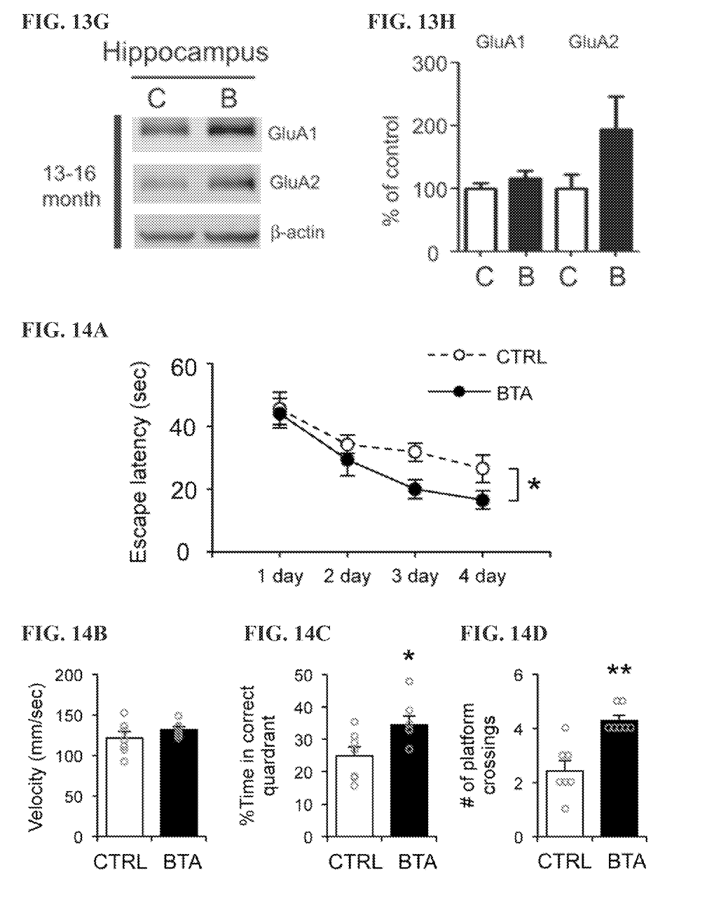

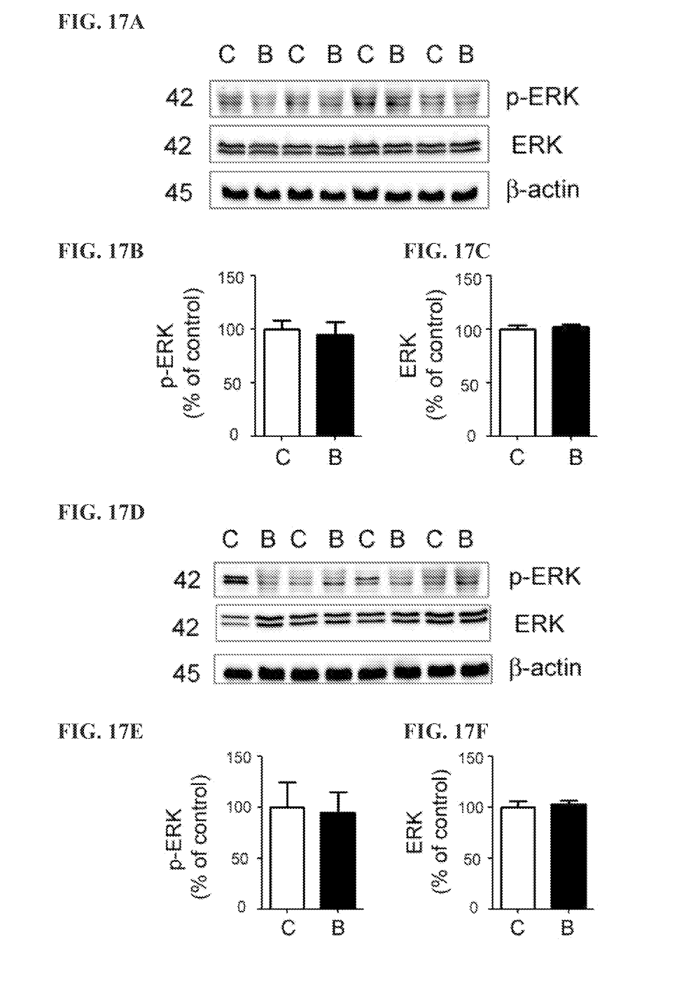

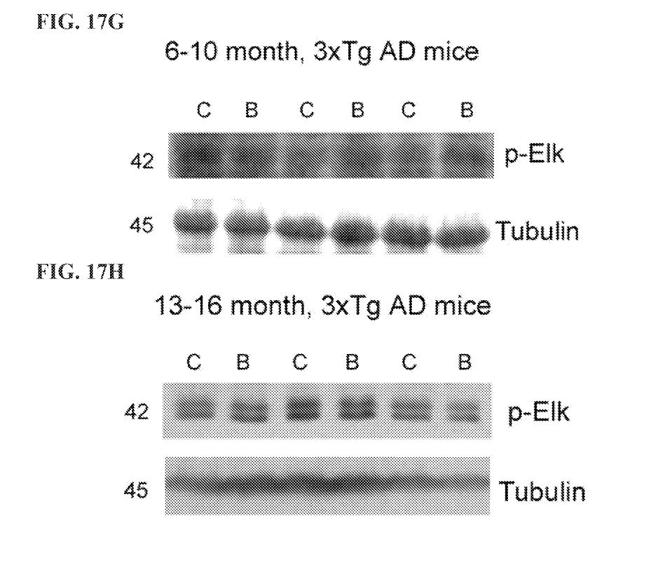

BTA-EG₄ injected 3× Tg AD mice had increased GluA2 levels. Next, we investigated whether BTA-EG₄ could alter downstream targets of the Ras signaling pathway. To test this, 6-10 month or 13-16 month old 3× Tg AD mouse brains were homogenized following BTA-EG₄ (30 mg/kg) or vehicle injection daily for 2 weeks. Using Western blots with specific GluA1 and GluA2 antibodies, we found an age-specific increase in the GluA2 subunit of AMPA receptors with BTA-EG₄ treatment. Specifically, GluA2 levels were increased in 6-10 month old 3× Tg AD mouse hippocampus without a significant change in the GluA1 levels (FIGS. 13C-13D, n=3-4 brains/group). There was only a trend of an increase in GluA2 in the cortex (FIGS. 13A-13B, n=3-4 brains/group). In the older 3× Tg AD mice (13-16 months old), there was no statistically significant change in the levels of AMPA receptor subunits in either brain area (FIGS. 13E-13H, n=3-4 brains/group). We then examined whether BTA-EG₄ injection could also alter the levels of p-ERK and p-Elk in 3× Tg AD mice. To test this, 6-10 month old or 13-16 month old 3× Tg AD mice were injected with 30 mg/kg BTA-EG₄ or vehicle daily for two weeks prior to measuring the levels of p-ERK and ERK. Unexpectedly, we found that BTA-EG₄ injected 6-10 month old 3× Tg AD mice did not have altered levels of p-ERK and ERK in the cortex and hippocampus (FIGS. 17A-17F). Moreover, we found that BTA-EG₄ injection in 6-10 month old 3× Tg AD mice or 13-16 month old 3× Tg AD mice did not alter the levels of p-Elk, which is a downstream target of phosphorylated ERK (FIGS. 17G-17H). Hence, the increases in AMPA receptor subunit GluA2 expression and Ras activity with BTA-EG₄ treatment correlate with the age-specific increases in dendritic spine density in a mouse model of AD.

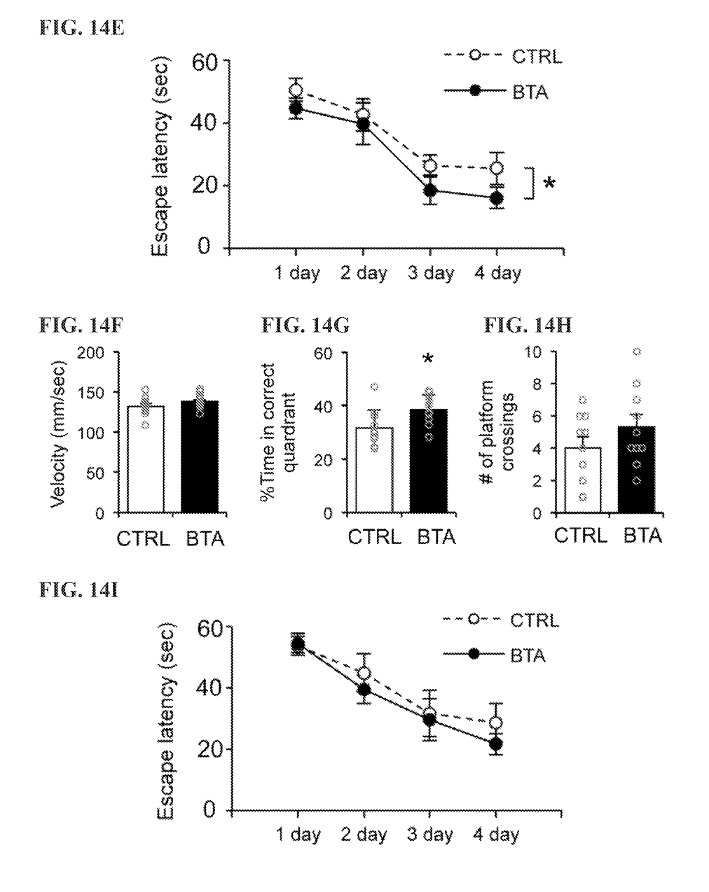

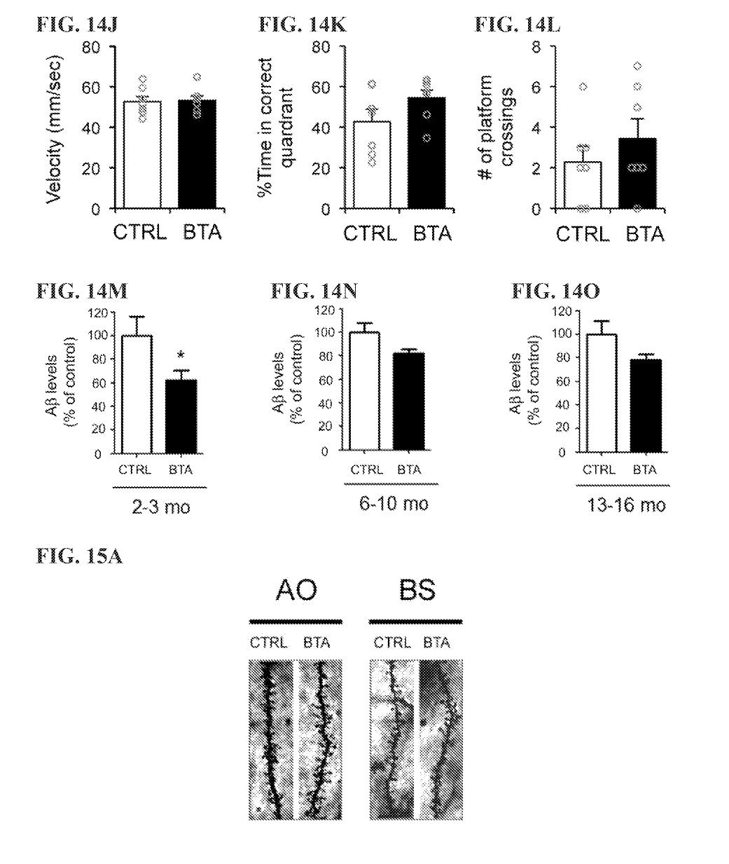

BTA-EG₄ improves learning and memory in 3× Tg AD mice. To examine whether BTA-EG₄ can improve learning and memory in a mouse model of AD, we injected 2-3 month old, 6-10 month old and 13-16 month old 3× Tg AD mice daily for 2 weeks with BTA-EG₄ (30 mg/kg) and conducted the Morris Water Maze. We found that there is an age-dependent progressive loss in the effectiveness of BTA-EG₄ in improving learning and memory of 3× Tg AD mice. As seen in FIGS. 14A-14D, 2-3 month old BTA-EG₄ injected mice demonstrated significantly faster escape latency during training trials and performed significantly better during probe trials, as seen by a greater percentage of time in the target quadrant and more platform crossings than control injected mice, without differing in swim speed (FIGS. 14A-14D, n=7/group). This behavioral improvement corresponds to a decrease in soluble Aβ 40 levels following BTA-EG₄ injection in 2-3 month old 3× Tg AD mice (FIG. 14M, n=7/group). At 6-10 months of age, BTA-EG₄ injected mice still demonstrated faster escape latency during training trials; however, during probe trials, only the percentage of time spent in the target quadrant was significantly improved without changes in the number of platform crossings (FIGS. 14E-14H, n=10/group) or soluble Aβ 40 level (FIG. 14N, n=9/group). By 13-16 months of age, none of the measured parameters were significantly altered in BTA-EG₄-injected mice (FIGS. 14I-14L, n=7/group, FIG. 14O, n=4-5/group). These data suggest that BTA-EG₄ improves both learning and memory on this standard spatial memory task, but the effectiveness of this drug is limited in older 3× Tg AD mice.

Discussion

This study demonstrates that BTA-EG₄ produces an age-specific improvement in synaptic density and cognitive function in a well established AD mouse model. In particular, we observed improvement in dendritic spine density accompanied by changes in dendritic spine morphology in cortical layers II/III and the CA1 region of the hippocampus in 3× Tg AD mice. Moreover, BTA-EG₄ increased Ras signaling and subsequent downstream signaling to synaptic AMPA receptors without altering phosphorylation of ERK and Elk in this mouse model, which was also most effective in young animals. Furthermore, we report that BTA-EG₄ is effective at improving memory-related cognitive function. However, the BTA-EG₄-induced improvement of synaptic loss and cognitive decline in the 3× Tg AD mice was most effective at ages before severe synapse loss.

In the present study, we selected a dosage of 30 mg/kg of BTA-EG₄ daily for 2 weeks due to its pronounced effect on dendritic spine density in wild-type mice (Megill et al., 2013). We found that dendritic spine density was increased in both the hippocampus CA1 region and cortical layers II/III at 6-10 months of age (mild Aβ plaque deposition and synapse loss) following BTA-EG₄ treatment in 3× Tg AD mice. While BTA-EG₄ was able to ameliorate dendritic spine loss typically seen in 13-16 month old (moderate Aβ plaque deposition and synapse loss) 3× Tg AD mice in cortical layers II/III, there was only a trend toward an increase in hippocampal spine density measured in CA1. This suggests that BTA-EG₄ is useful for improving early AD pathology. However, this limited effect may be due to the short (2-week) duration of BTA-EG₄ treatment; hence, it is possible that a longer treatment period or initiation of the treatment before severe Aβ plaque pathology may be more effective at improving dendritic spine density. Unexpectedly, we also found that dendritic spine density in 6-10 month old 3× Tg AD mice is higher than that of 2-3 month old 3× Tg AD mice. This might be due to either the normal function of APP before amyloid beta deposition that increases dendritic spine number, or the effect of tau increasing dendritic spine density on its own. Another possibility is that this may reflect normal development of dendritic spine density change, which is preserved in the 3× Tg AD mice. Future studies are warranted to examine these possibilities. Additionally, it will be of interest to investigate whether BTA-EG₄ has the same effect on dendritic spine density in other mouse models of AD, such as 2× Tg AD mice lacking tau pathology. Determining the effectiveness of BTA-EG₄ with and without tau pathology will be informative, especially in light of a recent study demonstrating that tau increases dendritic spine density while taumutants have reduced dendritic spine density (Kremer et al., 2011).

In addition to dendritic spine density, dendritic spine morphology analyses can elucidate the effects of treatment on synapse formation. For example, long and thin dendritic spines are often classified as “immature learning” spines, whereas short and wide dendritic spines are classified as “mature memory” spines (Kasai et al., 2002; Yasumatsu et al., 2008). Specifically, longer spines are thought of as substrates for conversion into mature spines via LTP-type mechanisms, while wider spines typically mediate stronger synaptic transmission (Matsuzaki et al., 2001). Previously, we reported that BTA-EG₄ injection does not alter dendritic spine morphology in wild-type mice (Megill et al., 2013). Yet, here we observed that BTA-EG₄ alters dendritic spine morphology in 3× Tg AD mice at 6-10 months of age. In particular, dendritic spines were longer and wider following daily BTA-EG₄ application for 2 weeks, suggesting that BTA-EG₄ can regulate dendritic spine structure. However, this effect was restricted in age, and we did not observe alterations in dendritic spine morphology in 13-16 month old 3× Tg AD mice following BTA-EG₄ treatment. This further corroborates the idea that BTA-EG₄ may be effective at altering dendritic spine morphology before high Aβ plaque load in this AD mouse model. Alternatively, a longer duration of treatment may be needed for altering dendritic spine morphology in aged AD mice with heavier plaque load. AD patients and mouse models of AD undergo decreased synaptic connectivity and increased synaptic loss with age (Knobloch and Mansuy, 2008; Scheff and Price, 2006). Because 13-16 month old 3× Tg AD mice have a greater loss of synapses than 6-10 month old mice, it may be more difficult to improve synapse number with only 2 weeks of BTA-EG₄ treatment.

We recently demonstrated that BTA-EG₄ promotes dendritic spine density through a full length APP and Ras-dependent mechanism in wild-type mice (Megill et al., 2013). Additionally, a recent study demonstrates that Aβ (in contrast to the effect of full length APP) decreases dendritic spine density by inhibiting Ras activity (Szatmari et al., 2013). Here, we found that Ras activity is increased following BTA-EG₄ injection in 6-10 month old, but not 13-16 month old, 3× Tg AD mice. We also found that BTA-EG₄ can selectively increase GluA2 levels while GluAl levels remained comparable to controls. It is known that Ras activity can regulate AMPA receptor expression (Gu and Stornetta, 2007; Qin et al., 2005), and in particular GluA2 subunit expression has been shown to increase dendritic spine density via its extracellular domain (Passafaro et al., 2003). Surprisingly, BTA-EG₄ injections in 6-10 month old 3× Tg AD mice did not alter downstream targets of Ras, such as p-ERK and p-Elk. This is opposite to the effects of BTA-EG₄ in wild-type mice in which downstream Ras targets were increased. This discrepancy may be explained by the complexities of Ras signaling in various circumstances, including localization of Ras isoforms and the disease state of the organism. It is known that Ras activates ERK in an isoform-specific manner (Prior and Hancock, 2012), and thus, BTA-EG₄ may act differentially to promote dendritic spine density in wild-type mice (the normal condition) and in a mouse model of AD (the pathological condition). One possibility may be that BTA-EG₄ upregulates an isoform of Ras through RasGRF1 that poorly activates ERK (and its downstream target Elk) in 3× Tg AD mice, as opposed to the effects of BTA-EG₄ in wild-type mice (to increase both Ras and ERK). One candidate Ras isoform activated by RasGRF1 is localized to the endoplasmic reticulum and has been shown to activate ERK less efficiently than other Ras isoforms (Matallanas et al., 2006). Thus, this isoform may be capable of regulating glutamate receptor insertion at the synapse in a manner that does not rely on ERK upregulation in 3× Tg mice. In any case, it is likely that BTA-EG₄ promotes dendritic spine density through enhancing Ras activity and increasing GluA2 AMPA receptor subunit expression in 3× Tg AD mice. Alternatively, BTA-EG₄ may also exert its effect by neutralizing Aβ, which has been shown to induce removal of synaptic AMPARs and reduce the density of dendritic spines (Hsieh et al., 2006; Kamenetz et al., 2003). These two possibilities are not mutually exclusive, and it is possible that the combination of increasing Ras signaling and blocking Aβ signaling may be responsible for the improvement in dendritic spine density in 3× Tg AD mice with BTA-EG₄ treatment.

We further found that BTA-EG₄ improves learning and memory in 3× Tg AD mice. This effect is similar to what we observed in wild-type mice (Megill et al., 2013), but with subtle differences. In wild-type mice, BTA-EG₄ mainly improved memory with little improvement on learning (Megill et al., 2013). However, in 3× Tg AD mice, both learning and memory are improved by BTA-EG₄. In particular, this improvement was only significant in the 2-3 month old and 6-10 month old, but not in the 13-16 month old, 3× Tg AD mice. We further found that BTA-EG₄ improved cognitive performance that correlated with decreased Soluble Aβ 40 levels in 2-3 month old 3× Tg AD mice, along with a trend toward a decrease at 6-10 months and 13-16 months of age. The age dependence of the effectiveness BTA-EG₄ mirrors that seen with dendritic spine improvement and Ras signaling. As discussed above, it would be of interest to investigate whether a longer treatment period or initiation of the treatment before Aβ plaque accumulation could be more effective at recovering behavioral performance. In either case, our current data strongly suggest that BTA-EG₄ treatment may be useful for the prevention of early AD pathology.

Conclusions

Our study demonstrates that BTA-EG₄ treatment can increase dendritic spine density in a mouse model of AD with mild and moderate Al3 plaque deposition and synapse loss. This change in dendritic spine 112 J. M. Song et al./Experimental Neurology 252 (2014) 105-113 density was associated with increased Ras activity. Moreover, we observed that BTA-EG₄ injected mice show improvement in learning and memory up to 6-10 months of age. Taken together, these findings suggest that BTA-EG₄ may be a beneficial therapy for preventing and/or treating the synaptic loss accompanying AD.

### REFERENCES

Capule, C. C. & Yang, J., 2012, Anal. Chem. 84:1786-1791.

Finder, V. H. & Glockshuber, R., 2007, Neurodegener Dis 4:13-27.

Gu, Y. & Stornetta, R. L., 2007, Acta Pharmacol. Sin. 28:928-936.

Habib, L. K., et al., 2010, J. Biol. Chem. 285:38933-38943.

Hsieh, H., et al., 2006, Neuron 52:831-843.

Inbar, P., et al., 2006, Chembiochem 7:1563-1566.

Kamenetz, F., et al., 2003, Neuron 37:925-937.

Kasai, H., et al., 2002, Nihon Shinkei Seishin Yakurigaku Zasshi 22:159-164.

Knobloch, M. & Mansuy, I. M., 2008, Mol. Neurobiol. 37:73-82.

Kremer, A., et al., 2011, J. Neurosci. 31:18036-18047.

Lustbader, J. W., et al., 2004, Science 304:448-452.

Masliah, E., et al., 2006, J. Alzheimers Dis. 9:91-99.

Matallanas, D., et al., 2006, Mol. Cell Biol. 26:100-116.

Matsuzaki, M., et al., 2001, Nat. Neurosci. 4:1086-1092.

Megill, A., et al., 2013, J. Neurosci. 33:9306-9318.

Minami, S. S., et al., 2012, Neurobiol. Aging 33(4):825.e15-24.

Oddo, S., et al., 2003, Neuron 39:409-421.

Passafaro, M., et al., 2003, Nature 424:677-681.

Prior, I. A. & Hancock, J. F., 2012, Semin. Cell Dev. Biol. 23:145-153.

Qin, Y., et al., 2005, Genes Dev. 19:2000-2015.

Scheff, S. W. & Price, D. A., 2006, J. Alzheimers Dis. 9:01-115.

Scheff, S. W., et al., 2006, Neurobiol. Aging 27:1372-1384.

Scheff, S. W., et al., 2007, Neurology: 1501-1508.

Selkoe, D. J., 2002, Science 298:789-791.

Szatmari, E. M., et al., 2013, J. Neurosci. 33:5367-5374.

Terry, R. D., et al., 1991, Ann. Neurol. 30:572-580.

Yasumatsu, N., et al., 2008, J. Neurosci. 28:13592-13608.

### IV. EMBODIMENTS

A first set of embodiments P1-P4 follows:

Embodiment P1. A method for improving memory and learning in a subject in need thereof, comprising administering to said subject a compound with structure of Formula (I):

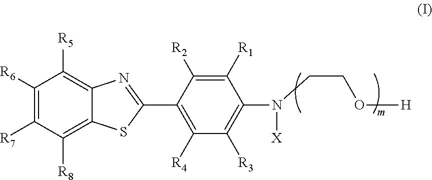

wherein R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl, m is a integer in the range 1-20, and X is hydrogen, methyl, or ethyl.

Embodiment P2. The method of embodiment P1, wherein said compound is

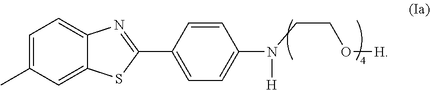

Embodiment P3. A method for treating cognitive impairment in a subject in need thereof, comprising administering to said subject a compound with structure of Formula (I):

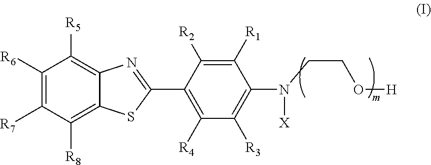

wherein R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl, m is a integer in the range 1-20, and X is hydrogen, methyl, or ethyl.

Embodiment P4. The method of embodiment P3, wherein said compound is

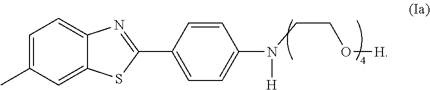

Further embodiments include the following:

Embodiment 1. A method for improving memory or learning in a subject in need thereof, the method including administering to the subject an effective amount of a compound of Formula (I):

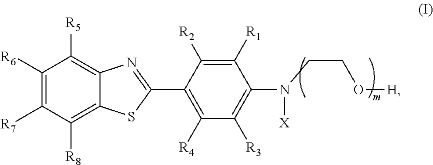

wherein R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl; m is a integer in the range 1-20; and X is hydrogen, methyl, or ethyl.

Embodiment 2. The method of embodiment 1, wherein the compound is

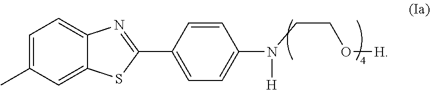

Embodiment 3. A method for treating neuronal or cognitive impairment in a subject in need thereof, the method including administering to the subject an effective amount of a compound of Formula (I):

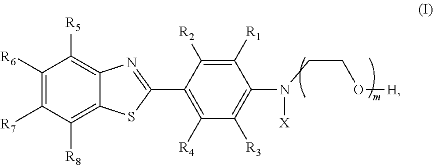

wherein R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl; m is a integer in the range 1-20; and X is hydrogen, methyl, or ethyl.

Embodiment 4. The method of embodiment 3, wherein the compound is

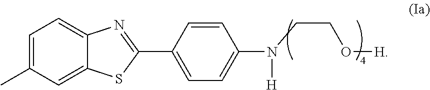

Embodiment 5. A method of increasing dendritic spine formation, increasing dendritic spine density or improving dendritic spine morphology in a subject in need thereof, the method including administering to the subject an effective amount of a compound of Formula (I):

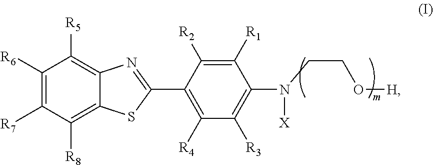

wherein R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl; m is a integer in the range 1-20; and X is hydrogen, methyl, or ethyl.

Embodiment 6. A method of increasing functional synapses in a subject in need thereof, the method including administering to the subject an effective amount of a compound of Formula (I):

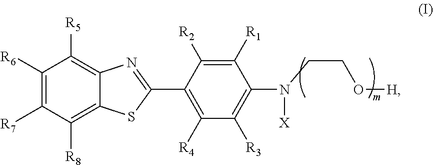

wherein R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl; m is a integer in the range 1-20; and X is hydrogen, methyl, or ethyl.

Embodiment 7. The method of one of embodiments 1-6, wherein the subject has Alzheimer's Disease.

Embodiment 8. The method of embodiment 7, wherein the method improves memory and learning in the subject.

Embodiment 9. The method of embodiment 7, wherein the subject has low Aβ plaque accumulation in the brain relative to an amount of Aβ plaque accumulation in an Alzheimer's disease standard control.

Embodiment 10. The method of one of embodiments 1-6, wherein the subject does not have Alzheimer's Disease.

Embodiment 11. The method of embodiment 9, wherein the method improves memory.

Embodiment 12. The method of one of embodiments 1-11, wherein said compound is administered to the subject daily for more than two weeks.

1. A method for improving memory or learning in a subject in need thereof, the method comprising administering to said subject an effective amount of a compound of Formula (I): 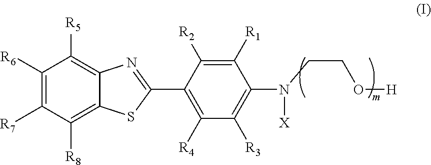
wherein
R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl,
m is a integer in the range 1-20, and
X is hydrogen, methyl, or ethyl.

2. The method of claim 1, wherein said compound is

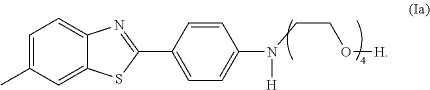

3. A method for treating neuronal or cognitive impairment in a subject in need thereof, the method comprising administering to said subject an effective amount of a compound of Formula (I): 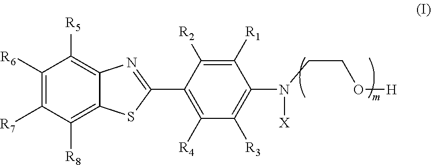
wherein
R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl,
m is a integer in the range 1-20, and
X is hydrogen, methyl, or ethyl.

4. The method of claim 3, wherein said compound is

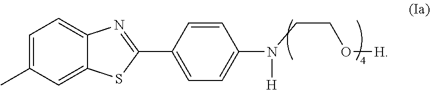

5. A method of increasing dendritic spine formation, increasing dendritic spine density or improving dendritic spine morphology in a subject in need thereof, said method comprising administering to said subject an effective amount of a compound of Formula (I): 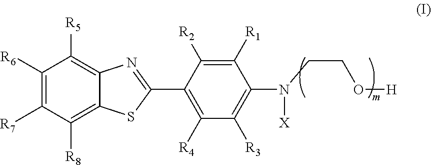
wherein
R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl,
m is a integer in the range 1-20, and
X is hydrogen, methyl, or ethyl.

6. A method of increasing functional synapses in a subject in need thereof said method comprising administering to said subject an effective amount of a compound of Formula (I): 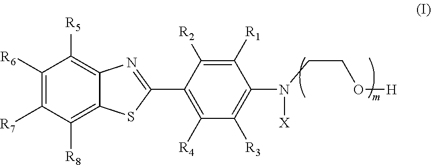
wherein
R₁-R₈ are selected from the group consisting of hydrogen, deuterium, tritium, fluoride, chloride, bromide, iodide, hydroxide, amino, methylamino, dimethylamino, trimethylammonium, methyl, ethyl, methoxy, ethoxy, fluoromethyl, difluoromethyl and trifluoromethyl,
m is a integer in the range 1-20, and
X is hydrogen, methyl, or ethyl.

7. The method of claim 1, wherein said subject has Alzheimer's Disease.

8. The method of claim 7, wherein said method improves memory and learning in said subject.

9. The method of claim 7, wherein said subject has low Aβ plaque accumulation in the brain relative to an amount of Aβ plaque accumulation in an Alzheimer's disease standard control.

10. The method of claim 1, wherein said subject does not have Alzheimer's Disease.

11. The method of claim 9, wherein said method improves memory.

12. The method of claim 1, wherein said compound is administered to said subject daily for more than two weeks.
